(19) United States
(12) Patent Application Publication
(10) Pub. No.: US 2026/0199627 A1
(43) Pub. Date: Jul. 16, 2026

(54) DEVICES, SYSTEMS, AND METHODS FOR DETECTION OF MEDICAL DEVICE

(71) Applicant: RESMED PTY LTD, Bella Vista, New South Wales (AU)

(72) Inventors: David Noel CREUSOT, Sydney (AU); Michael James DENT, Sydney (AU); Dmitri Anatolievich DOUDKINE, Sydney (AU); Samuel Thomas HORLER, Sydney (AU); Ting Lee TEH, Sydney (AU); Rudi VOON, Sydney (AU)

(21) Appl. No.: 19/135,649

(22) PCT Filed: Dec. 13, 2023
(86) PCT No.: PCT/AU2023/051292

§ 371 Date: Jun. 4, 2025

Related U.S. Application Data
(60) Provisional application No. 63/387,325, filed on Dec. 14, 2022.
(60) Provisional application No. 63/487,311, filed on Feb. 28, 2023.
(60) Provisional application No. 63/487,317, filed on Feb. 28, 2023.
(60) Provisional application No. 63/515,681, filed on Jul. 26, 2023.

(51) Int. Cl. A61M 16/08; A61M 16/00; A61M 16/10

(52) U.S. Cl. CPC A61M 16/0816; A61M 16/0069; A61M 16/024; A61M 2016/0027; A61M 2016/103; A61M 2205/3331; A61M 2205/3592; A61M 2205/60; A61M 2230/62; A61M 2230/63

(57) ABSTRACT

An air adapter tube for diagnosis and/or treatment of a respiratory disorder includes a tubular body configured to transport pressurized treatment air between a proximal end of the tubular body and a distal end of the tubular body, the proximal end being configured to connect to a patient interface, and the distal end being configured for connection to an air delivery tube; and a sensor being configured to generate a signal based on air passing at the proximal end for diagnosis and/or treatment of a respiratory disorder.

### 1 CROSS-REFERENCE TO RELATED APPLICATIONS

This application claims priority to U.S. Provisional Application Nos. 63/387,325, filed Dec. 14, 2022, 63/487,311, filed Feb. 28, 2023, 63/487,317, filed Feb. 28, 2023, and 63/515,681, filed Jul. 26, 2023, each of which is incorporated herein by reference in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices, or apparatus, systems, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 2.2.3.1.2 Positioning and Stabilising

A seal-forming structure of a patient interface used for positive air pressure therapy is subject to the corresponding force of the air pressure to disrupt a seal. Thus, a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus, RPT devices may also act as flow therapy devices. Examples of RPT devices include a CPAP device and a ventilator.

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

An example of the special requirements of certain RPT devices is acoustic noise.

Table of noise output levels of prior RPT devices
(one specimen only, measured using test method
specified in ISO 3744 in CPAP mode at 10 cmH2O).

| | A-weighted sound | Year |
|---|---|---|
| RPT Device name | pressure level dB(A) | (approx.) |
| C-Series TangoTM | 31.9 | 2007 |
| C-Series TangoTM with Humidifier | 33.1 | 2007 |
| S8 EscapeTM II | 30.5 | 2005 |
| S8 EscapeTM II with H4iTM | 31.1 | 2005 |
| Humidifier | | |
| S9 AutoSetTM | 26.5 | 2010 |
| S9 AutoSetTM with H5i Humidifier | 28.6 | 2010 |

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

A range of artificial humidification devices and systems are known, however they may not fulfil the specialised requirements of a medical humidifier.

Medical humidifiers are used to increase humidity and/or temperature of the flow of air in relation to ambient air when required, typically where the patient may be asleep or resting (e.g. at a hospital). A medical humidifier for bedside placement may be small. A medical humidifier may be configured to only humidify and/or heat the flow of air delivered to the patient without humidifying and/or heating the patient's surroundings. Room-based systems (e.g. a sauna, an air conditioner, or an evaporative cooler), for example, may also humidify air that is breathed in by the patient, however those systems would also humidify and/or heat the entire room, which may cause discomfort to the occupants. Furthermore, medical humidifiers may have more stringent safety constraints than industrial humidifiers.

While a number of medical humidifiers are known, they can suffer from one or more shortcomings. Some medical humidifiers may provide inadequate humidification, some are difficult or inconvenient to use by patients.

### 2.2.3.5 Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched gas are condensed. POCs seek to utilize their produced oxygen as efficiently as possible, in order to minimise weight, size, and power consumption. This may be achieved by delivering the oxygen as series of pulses, each pulse or “bolus” timed to coincide with the onset of inhalation. This therapy mode is known as pulsed oxygen delivery (POD) or demand mode, in contrast with traditional continuous flow delivery more suited to stationary oxygen concentrators.

### 2.2.3.6 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.7 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

Table of noise of prior masks (ISO 17510-2:
2007, 10 cmH2O pressure at 1 m)

| | | A-weighted | A-weighted | |
|---|---|---|---|---|
| | | sound power | sound | |
| | Mask | level dB(A) | pressure dB(A) | Year |
| Mask name | type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| MirageTM (*) | | | | |
| ResMed | nasal | 36 (3) | 28 (3) | 2000 |
| UltraMirageTM | | | | |
| ResMed Mirage | nasal | 32 (3) | 24 (3) | 2002 |
| ActivaTM | | | | |
| ResMed Mirage | nasal | 30 (3) | 22 (3) | 2008 |
| MicroTM | | | | |
| ResMed MirageTM | nasal | 29 (3) | 22 (3) | 2008 |
| SoftGel | | | | |
| ResMed MirageTM | nasal | 26 (3) | 18 (3) | 2010 |
| FX | | | | |
| ResMed Mirage | nasal | 37 | 29 | 2004 |
| SwiftTM (*) | pillows | | | |
| ResMed Mirage | nasal | 28 (3) | 20 (3) | 2005 |
| SwiftTM II | pillows | | | |
| ResMed Mirage | nasal | 25 (3) | 17 (3) | 2008 |
| SwiftTM LT | pillows | | | |
| ResMed | nasal | 21 (3) | 13 (3) | 2014 |
| AirFit P10 | pillows | | | |

(* one specimen only, measured using test method specified in ISO 3744 in CPAP mode at 10 cmH2O)

Sound pressure values of a variety of objects are listed below:

| | A-weighted sound | |
|---|---|---|
| Object | pressure dB(A) | Notes |
| Vacuum cleaner: Nilfisk | 68 | ISO 3744 at |
| Walter Broadly Litter | | 1 m distance |
| Hog: B+ Grade | | |
| Conversational speech | 60 | 1 m distance |
| Average home | 50 | |
| Quiet library | 40 | |
| Quiet bedroom at night | 30 | |
| Background in TV studio | 20 | |

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a respiratory therapy system, comprising: a patient interface; an air circuit configured to removably couple to the patient interface; a radio frequency identification (RFID) tag associated with the patient interface, the RFID tag being configured to store information about the patient interface; an antenna; and a transceiver, wherein the transceiver may be configured to wirelessly receive the information stored on the RFID tag when the air circuit may be coupled to the patient interface and to transmit the information to a controller.

In an aspect of the present technology, at least one of the antenna or the transceiver may be located on an adapter configured to couple the air circuit to the patient interface.

In another aspect, at least one of the antenna or the transceiver are located on a proximal end of the air circuit.

In one form, at least one of the antenna or the transceiver may be housed within a cover located at the proximal end of the air circuit.

In an aspect, the RFID tag may be one or more of a near-field communication (NFC) tag, an ultra-high frequency (UHF) tag, a Bluetooth tag, or an ultra wideband (UWB) tag.

In aspects, the controller may be incorporated as part of a respiratory pressure therapy device.

In one form, the controller may be configured to automatically configure at least one setting of the respiratory pressure therapy device based on the information received from the transceiver about the patient interface.

In aspects, the antenna may be a multi-directional antenna comprising a plurality of antennas oriented at different angles relative to one another.

In another form, an air circuit for a respiratory therapy system may comprise: a proximal end and a distal end, wherein the proximal end may be configured to removably couple to a patient interface, and the distal end may be configured to removably couple to a respiratory pressure therapy device; and a multi-dimensional antenna located at a proximal region of the air circuit, wherein the multi-dimensional antenna may comprise a plurality of antennas oriented at different angles relative to one another.

In aspects, the multi-dimensional antenna may comprise two antennas.

In forms, the two antennas may be oriented at right angles relative to one another.

In further aspects, the multi-dimensional antenna comprises three or more antennas.

In some forms, the plurality of antennas may comprise at least a first antenna and a second antenna, wherein the first antenna and the second antenna may be oriented at an angle of approximately 30 degrees to approximately 120 degrees relative to one another.

In aspects, the plurality of antennas may be set to a frequency of about 10 MHz to about 12 GHz.

In a further form, a respiratory therapy system may comprise: a respiratory pressure therapy device; and a controller, the controller configured to perform operations comprising: receiving information about a patient interface from an RFID tag associated with the patient interface; and performing a responsive action based at least in part on the received information about the patient interface, the responsive action comprising one or more of configuring a setting of the respiratory pressure therapy device; generating an indication that a new patient interface or a new cushion of the patient interface is recommended; generating an indication that a different size or a different type of the patient interface or cushion is recommended; generating an indication that an air circuit is not coupled to the patient interface or is improperly coupled to the patient interface; or transmitting the information about the patient interface to a cloud server.

In aspects, the controller may be incorporated as a part of the respiratory pressure therapy device.

In forms, the received information about the patient interface may comprise one or more of: a type or a size of the patient interface; a type or a size of the cushion; a production batch identification number or serial identification number of the patient interface; a time stamp; a date of usage; or a length of use of the patient interface.

In aspects, the information about the patient interface may be received at a start of a therapy session administered by the respiratory therapy system.

In further aspects, the information about the patient interface may be received at regular intervals throughout a therapy session administered by the respiratory therapy system.

In some forms, a frequency with which the information about the patient interface is received may increase if there is an irregularity detected in the information received.

Another aspect of one form of the present technology relates to an air adapter tube for diagnosis and/or treatment of a respiratory disorder. The air adapter tube also includes a tubular body configured to transport pressurized treatment air between a proximal end of the tubular body and a distal end of the tubular body, the proximal end being configured to connect to a patient interface, and the distal end being configured for connection to an air delivery tube; and a sensor being configured to generate a signal based on air passing at the proximal end for diagnosis and/or treatment of a respiratory disorder.

Another aspect of one form of the present technology relates to an air adapter tube for diagnosis and/or treatment of a respiratory disorder. The air adapter tube also includes a tubular body configured to transport pressurized treatment air between a proximal end of the tubular body and a distal end of the tubular body, the proximal end being configured to connect to a patient interface, and the distal end being configured for connection to an air delivery tube; and a sensor being configured to generate a signal indicative of sleeping position of a patient.

Another aspect of one form of the present technology relates to an air adapter tube for diagnosis and/or treatment of a respiratory disorder. The air adapter tube also includes a tubular body configured to transport pressurized treatment air between a proximal end of the tubular body and a distal end of the tubular body, the proximal end being configured to connect to a patient interface, and the distal end being configured for connection to an air delivery tube; and a sensor being configured to generate a signal indicative of the breath of and/or a biomarker of gas exhaled by the patient.

Another aspect of one form of the present technology relates to an air adapter tube for diagnosis and/or treatment of a respiratory disorder. The air adapter tube also includes a proximal end and a distal end, where the proximal end is configured to removably couple to a patient interface, and the distal end is configured to removably couple to an air delivery tube; a sensor configured to generate a signal based on a sensed physical quantity, and an antenna configured to receive data from an RFID tag.

In an example, the sensor includes a pressure sensor.

In an example, the sensor includes an accelerometer.

In an example, the sensor is a CO₂ sensor.

In an example, the CO₂ sensor is configured to detect CO₂ buildup, re-breathed CO₂ levels and/or breathing comfort.

In an example, the CO₂ sensor is configured to be arranged between the patient and a vent on the patient interface.

In an example, the CO₂ sensor is configured to detect end-tidal CO₂.

In an example, the sensor is a volatile organic compounds (VOC) sensor configured to detect biomarkers within the patient's breath.

In an example, the sensor is a CO₂ sensor and/or a volatile organic compounds (VOC) sensor, and wherein the CO₂ sensor and/or the (VOC) sensor is configured to conduct pre-therapy analysis before pressurized treatment air is delivered to the patient interface.

In an example, the tubular body comprises a flexible material.

In an example, an internal diameter of the tubular body is between about 10-15 mm.

In an example, the internal diameter of the tubular body is 12 mm.

In an example, a length of the tubular body is between about 8.5-10 cm.

In an example, the length is about 8.5 cm.

In an example, an exterior surface of the tubular body comprises or is covered with textile material.

In an example, the tubular body includes a helix.

In an example, the proximal end includes a mechanical connector configured to connect to the patient interface.

In an example, the mechanical connector comprises a circlip with a pair of opposed clips configured to be connected to the patient interface.

In an example, the mechanical connector includes an iso-taper configured to connect with the patient interface with an interference or frictional fit.

In an example, the air adapter tube further includes a flexible printed circuit at the proximal end supporting the sensor.

In an example, the flexible circuit includes an antenna, which may be a NFC antenna.

In an example, the antenna is configured to wirelessly transmit collected sensor data to an external device, including a smart phone or a flow generator.

In an example, the flexible printed circuit includes at least one wire connection point that connects the antenna to at least one wire extending along and from the tubular body.

In an example, the flexible printed circuit includes linear sections to support electronic components and at least one flexible bent portion between the linear sections.

In an example, one of the electronic components is a pressure sensor, and another of the electronic components is an accelerometer sensor configured to generate a signal indicative of a patient's sleeping position.

In an example, the electronic components include one or more sensors configured to generate a signal representative of a breath and/or a biomarker indicative of health of a patient.

In an example, the one or more sensors include a CO₂ sensor or a volatile organic compounds (VOC) sensor.

In an example, the air adapter tube further includes a temperature sensor configured to generate a signal indicative of temperature inside the tubular body.

In an example, the air adapter tube further includes a first adapter element at the proximal end, the first adapter element supporting the sensor.

In an example, the first adapter element includes an outer cylindrical surface with a port, and the sensor protrudes radially inwards into and/or through the port.

In an example, the air adapter tube further includes silicone to seal the port adjacent the sensor.

In an example, the air adapter tube further includes a second adapter element that connects and seals with a film and helix of the tubular body.

In an example, the air adapter tube further includes a cuff portion connected to the first adapter element, and the first adapter element and the cuff portion form an annular space configured to receive a flexible printed circuit supporting the sensor.

In an example, the first adapter element includes at least one feature configured to support the flexible printed circuit, the at least one feature including a fastener and/or adhesive.

In an example, the air adapter tube further includes a vent hole along a perimeter of the first adapter element.

In an example, the air adapter tube further includes a water resistant pressure balancing membrane provided to the vent hole and configured to adjust differential pressure at the proximal end.

In an example, the air adapter tube further includes an electrical connector provided at the distal end of the tubular body, the electrical connector configured to electrically connect to a corresponding electrical connector of a heated air delivery tube.

In an example, the electrical connector comprises a leadframe configured to transmit power and/or signals.

In an example, the air adapter tube further includes an indicator or guide adjacent the distal end configured to align with a corresponding indicator or guide of the heated air delivery tube.

In an example, the air adapter tube further includes at least one wire extending along the tubular body and electrically connecting the electrical connector to the sensor and/or a flexible printed circuit.

In an example, the air adapter tube further includes a mechanical connector provided at the distal end of the tubular body, the mechanical connector configured to mechanically connect to a corresponding mechanical connector of the heated air delivery tube.

In an example, the air adapter tube further includes a power source configured to provide power to the sensor or a flexible printed circuit.

In an example, the air adapter tube further includes a plurality of sensors including at least a first sensor, a second sensor, and a third sensor. The first sensor is configured to generate, during therapy, a signal based on air passing at the proximal end for diagnosis and/or treatment of a respiratory disorder. The second sensor is configured to generate a signal indicative of sleeping position of a patient. The third sensor is configured to generate a signal indicative of the breath of and/or a biomarker of gas exhaled by the patient prior to therapy being applied.

In an example, the air adapter tube further includes a first electric connection configured to couple a first wire that is configured to deliver power to the air delivery tube and a second electric connection configured to couple to a second wire that is configured to send and receive data to/from the air delivery tube.

In an example, the antenna is a multi-directional antenna comprising a plurality of antennas oriented at different angles relative to one another.

In an example, the multi-directional antenna comprises two antennas.

In an example, the plurality of antennas comprises at least a first antenna and a second antenna, wherein the first antenna and the second antenna are oriented at an angle of approximately 30 degrees to approximately 120 degrees relative to one another.

In an example, the plurality of antennas are set to a frequency of about 10 MHz to about 12 GHz.

In an example, the sensor is supported at the proximal end of the air adapter tube and/or the tubular body thereof.

Another aspect of one form of the present technology relates to a medical treatment apparatus for diagnosing and/or treating a patient with a respiratory disorder including a flow generator configured to generate pressurized breathable air for the patient, a patient interface configured to seal with the patient's airways, an air delivery tube to deliver the pressurized breathable air from the flow generator to towards the patient interface, and an air adapter tube.

In an example, the flow generator is configured to be controlled based on output from the sensor.

Another aspect of one form of the present technology relates to a method for diagnosing and/or treating a patient including connecting an air adapter tube between an air delivery tube and a patient interface, generating a signal with a sensor, and transmitting the signal for use in diagnosing or treating the patient.

Another aspect of one form of the present technology relates to a system that comprises an air adapter tube and at least one hardware processor that is configured to perform operations comprising controlling when the sensor is configured to sense a physical quantity.

In an example, the sensor is controlled based on a determined position of a patient. In an example, the sensor is controlled to sense the physical quantity prior to positive air pressure being delivered to the air adapter tube.

In an example, the sensor is controlled to sense the physical quantity based on determination that there is no positive air pressure being delivered to the air adapter tube.

Another aspect of one form of the present technology relates to a system that comprises an air adapter tube and at least one hardware processor that is configured to perform operations comprising changing at least one therapy parameter based on the generated signal of the sensor.

In an example, the system further includes a patient interface that includes an RFID tag that includes the data received by the antenna of the air adapter tube.

The methods, systems, devices and apparatus described may be implemented to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the Figures of the accompanying drawings, in which like reference numerals refer to similar elements including:

FIGS. 1A-1C each illustrate various configurations of a respiratory therapy system in use.

FIG. 2 shows a patient interface in the form of a nasal mask in accordance with one form of the present technology.

FIG. 3A is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 3B is a schematic diagram of the electrical components of an RPT device in accordance with one form of the present technology.

FIG. 3C is a schematic diagram of the algorithms implemented in an RPT device in accordance with one form of the present technology.

FIG. 4A shows a perspective view of a cushion of a patient interface configured to be worn by a patient and convey pressurized air to the patient's nose and the patient's mouth.

FIG. 4B shows a perspective view of a cushion of a patient interface configured to be worn by a patient and convey pressurized air to the patient's nose.

FIG. 4C shows a perspective view of tubes usable with either the cushion of FIG. 4A or the cushion of FIG. 4B.

FIG. 4D shows a perspective view of rigidiser arms usable with either the cushion of FIG. 4A of the cushion of FIG. 4B.

FIG. 4E shows a perspective view of headgear straps usable with the cushion of FIG. 4A.

FIG. 4F shows a perspective view of headgear straps usable with the cushion of FIG. 4B.

FIG. 4G shows a front view of a pair of sleeves that is removably fitted to either the tubes of FIG. 4C or the rigidiser arms of FIG. 4D.

FIG. 4H shows a front view of a full sleeve that is removably fitted to the rigidiser arms of FIG. 4D.

FIG. 4I shows a front perspective view of yet another alternate form of a full sleeve that is removably fitted to the rigidiser arms of FIG. 4D.

FIG. 4J is a front view of a patient wearing the cushion of FIG. 4A connected to the tubes of FIG. 4C, the headgear straps of FIG. 4E, and the sleeves of FIG. 4G.

FIG. 4K is a front view of a patient wearing the cushion of FIG. 4A connected to the rigidiser arms of FIG. 4D, the headgear straps of FIG. 4E, and the sleeve of FIG. 4H.

FIG. 4L is a front view of a patient wearing the cushion of FIG. 4B connected to the conduit headgear of FIG. 4C, and the headgear straps of FIG. 4F.

FIG. 4M is a front view of a patient wearing the cushion of FIG. 4B connected to the rigidisier arms of FIG. 4D, the headgear straps of FIG. 4F, and the sleeve of FIG. 4I.

FIG. 4N is an isolated perspective view of the vent of FIG. 4L.

FIG. 4O is an isolated perspective view of a portion of the air circuit of FIG. 4M.

FIG. 4P is a schematic view illustrating the possible combinations of the patient interfaces.

FIG. 5A illustrates a schematic view of a medical system, according to aspects of this disclosure.

FIG. 5B illustrates a schematic view of an alternative medical system, according to aspects of this disclosure.

FIGS. 6A and 6B illustrate perspective views of a proximal portion of an air circuit, according to aspects of this disclosure.

FIG. 6C illustrates a front view of a circuit board shown in FIG. 6B, according to aspects of this disclosure.

FIGS. 7A and 7B illustrate a front view (FIG. 7A) and a side view (FIG. 7B) of an exemplary patient interface, according to aspects of this disclosure.

FIGS. 8A and 8B illustrate a back view (FIG. 8A) and a top view (FIG. 8B) of an alternative exemplary patient interface, according to aspects of this disclosure.

FIGS. 9A-9D illustrate various configurations of two or more antennas, according to aspects of this disclosure.

FIGS. 10A-10D illustrate a variety of configurations between a variety of patient interfaces and an air circuit.

FIG. 11 is a perspective view of an air adapter tube according to an example of the present technology, as viewed from a distal end.

FIG. 12 is another perspective view of the air adapter tube of FIG. 11 as viewed from the distal end.

FIG. 13 is another perspective view of the air adapter tube of FIG. 11 as viewed from a proximal end.

FIG. 14 is another perspective view of the air adapter tube of FIG. 11 as viewed from the proximal end.

FIG. 15 is a perspective view of the air adapter tube of FIG. 11 being connected to an air delivery tube according to an example of the present technology.

FIG. 16 is another perspective view of the air adapter tube of FIG. 11 being connected to an air delivery tube according to an example of the present technology.

FIG. 17 is a perspective view of the air adapter tube of FIG. 11 connected to an air delivery tube according to an example of the present technology.

FIG. 18 is another perspective view of the air adapter tube of FIG. 11 connected to an air delivery tube according to an example of the present technology.

FIG. 19 is another perspective view of the air adapter tube of FIG. 11 connected to an air delivery tube according to an example of the present technology.

FIG. 20 is an exploded view of the air adapter tube of FIG. 11 according to an example of the present invention.

FIG. 21 is another exploded view of the air adapter tube of FIG. 11 according to an example of the present invention.

FIG. 22 is a partial cross-sectional view of the air adapter tube of FIG. 11 according to an example of the present invention.

FIG. 23 is an exploded, partial cross-sectional view of the air adapter tube of FIG. 11 according to an example of the present invention.

FIG. 24 is another exploded, partial cross-sectional view of the air adapter tube of FIG. 11 according to an example of the present invention.

FIG. 25 is an exploded view of an end of an air delivery tube according to an example of the present technology.

FIG. 26 is another exploded view of the end of an air delivery tube of FIG. 25 according to an example of the present technology.

FIG. 27 is a perspective view of a proximal end of the air adapter tube of FIG. 11 with a cuff portion removed according to an example of the present technology.

FIG. 28 is another perspective view of a proximal end of the air adapter tube of FIG. 11 with a cuff portion removed according to an example of the present technology.

FIG. 29 is another perspective view of a proximal end of the air adapter tube of FIG. 11 with a cuff portion removed according to an example of the present technology.

FIG. 30 is an end view of a proximal end of the air adapter tube of FIG. 11 with a cuff portion removed according to an example of the present technology.

FIG. 31 is a partial cross-sectional view of the air adapter tube of FIG. 11 according to an example of the present invention.

FIG. 32 is a cross-sectional view of the air adapter tube of FIG. 11 with a cuff portion removed according to an example of the present invention.

FIG. 33 is a perspective view of the air adapter tube of FIG. 11 connected to an air delivery tube according to an example of the present technology.

FIG. 34 is a cross-sectional view through line 34-34 of FIG. 33.

FIG. 35 is a cross-sectional view through line 35-35 of FIG. 33.

FIG. 36 is a cross-sectional view of the air adapter tube of FIG. 11 connected to a patient interface according to an example of the present technology.

FIG. 37 is a cross-sectional view of the air adapter tube of FIG. 11 being manually disconnected from a patient interface according to an example of the present technology.

FIG. 38 is a plan view of a flexible printed circuit according to an example of the present technology.

FIG. 39 is a block diagram of the air adapter tube of FIG. 11 according to an example of the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user (e.g., patient). By contrast, the term “proximal” refers to a portion closest to the user.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

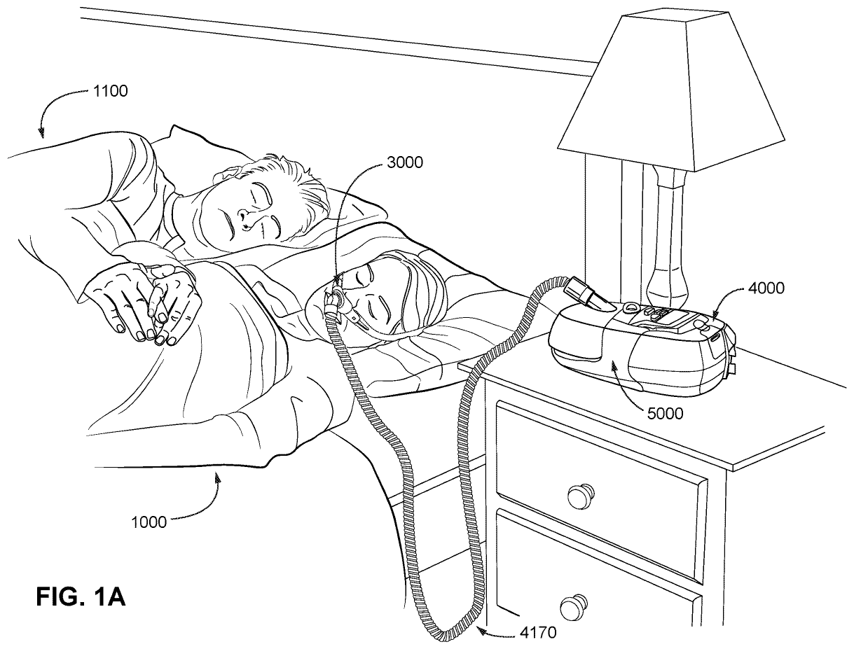

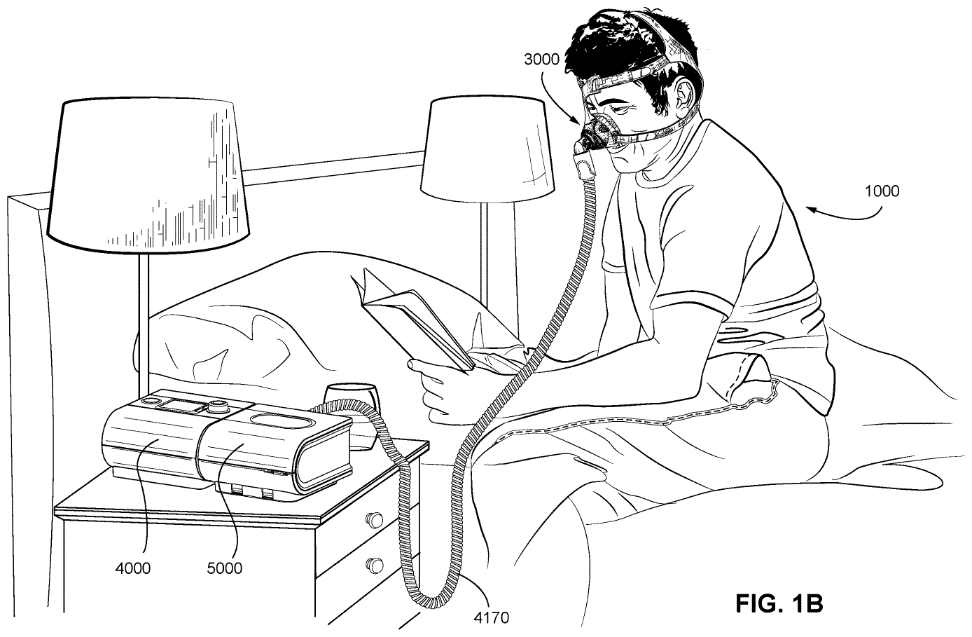

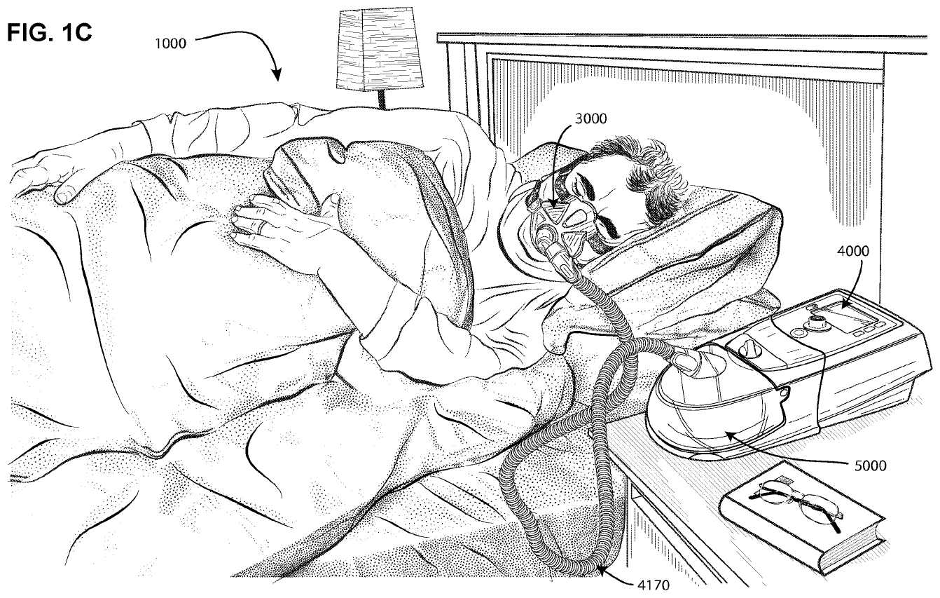

FIG. 1A shows a respiratory therapy system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows an alternative configuration of the respiratory therapy system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a further alternative configuration of the respiratory therapy system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10cmH2O, or at least 20 cmH2O.

As shown in FIGS. 1A, 1B, and 1C, patient 1000 may use the respiratory therapy system in a variety of positions. Accordingly, a position of patient interface 3000, for example, relative to air circuit 4170 or other aspects of the respiratory therapy system may vary throughout use.

### 5.3 Patient Interface

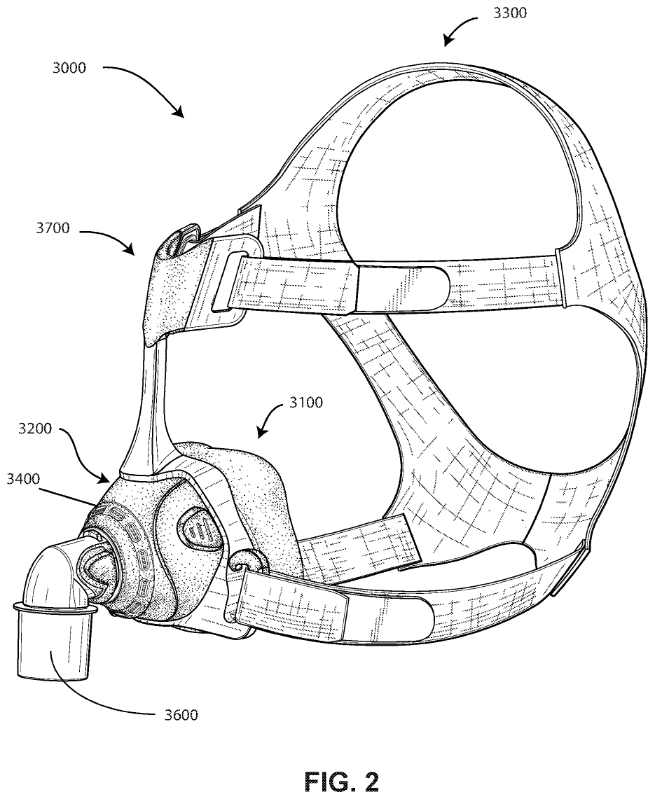

A non-invasive patient interface 3000, such as that shown in FIG. 2, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

### 5.3.1 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.4 MPa or less, for example between 0.4 MPa and 0.3 MPa. An example of such a material is silicone.

### 5.3.1.1 Multiple Openings

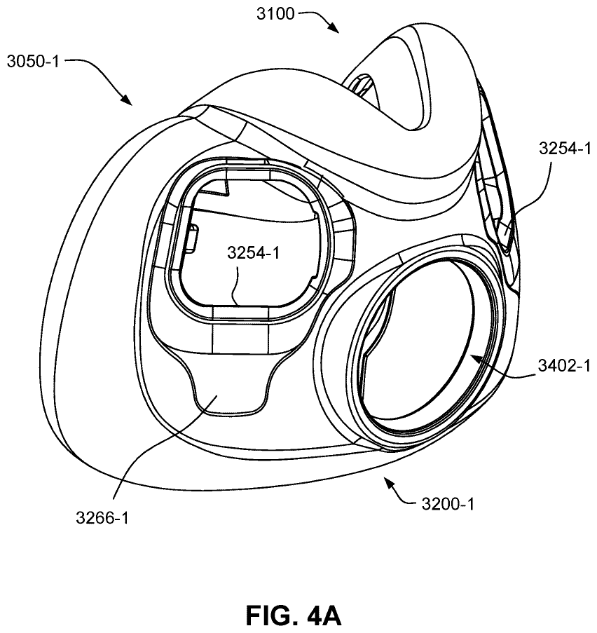

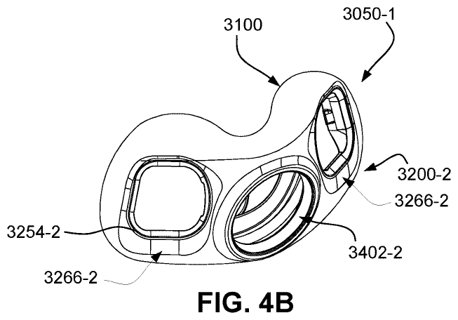

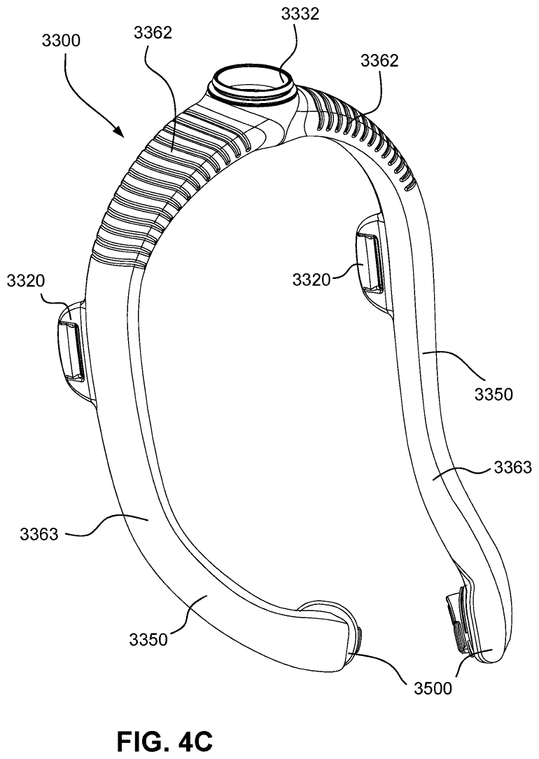

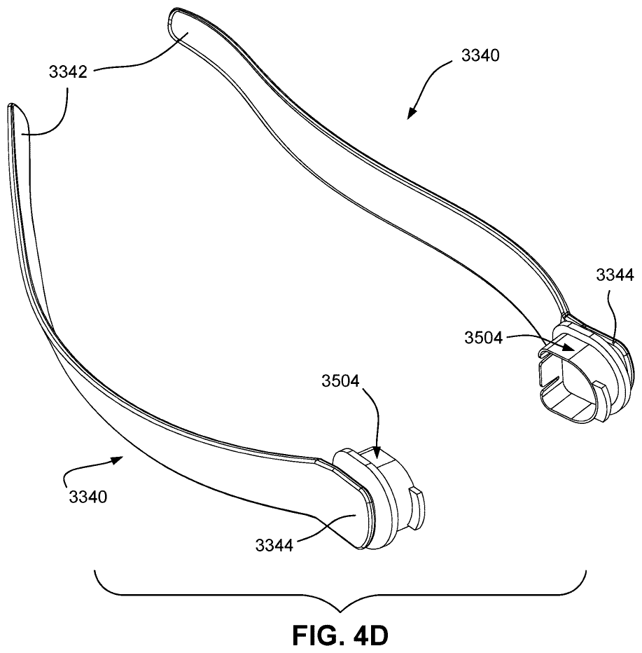

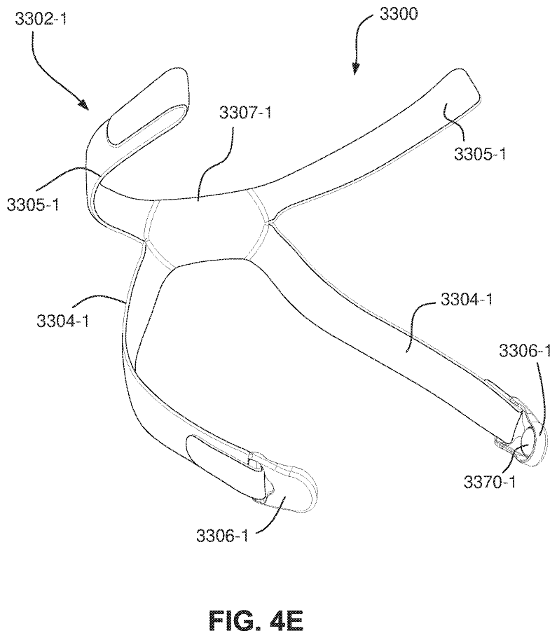

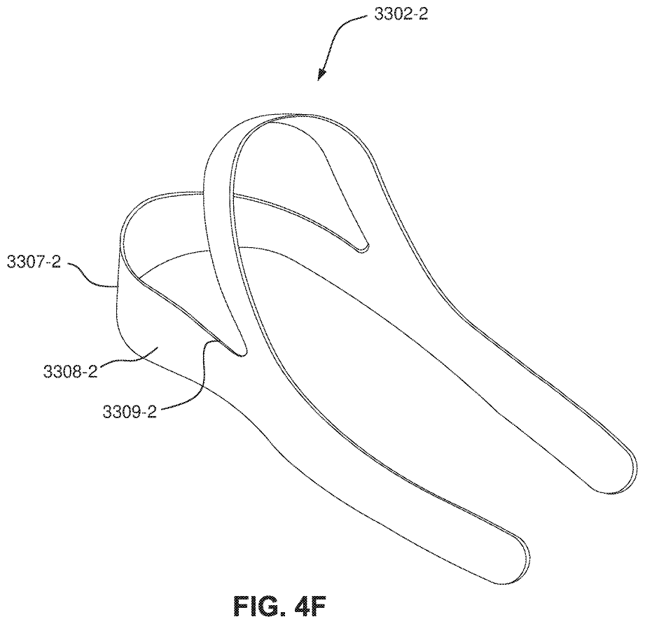

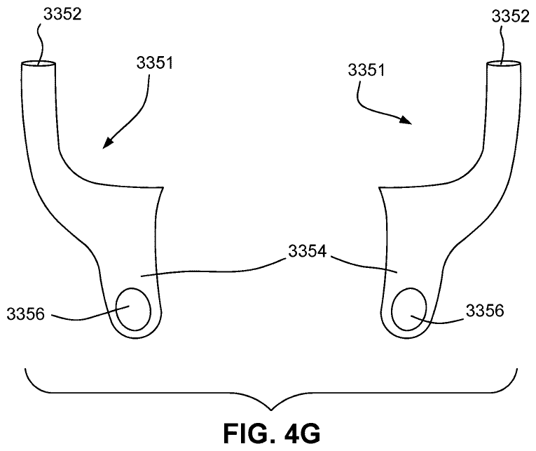

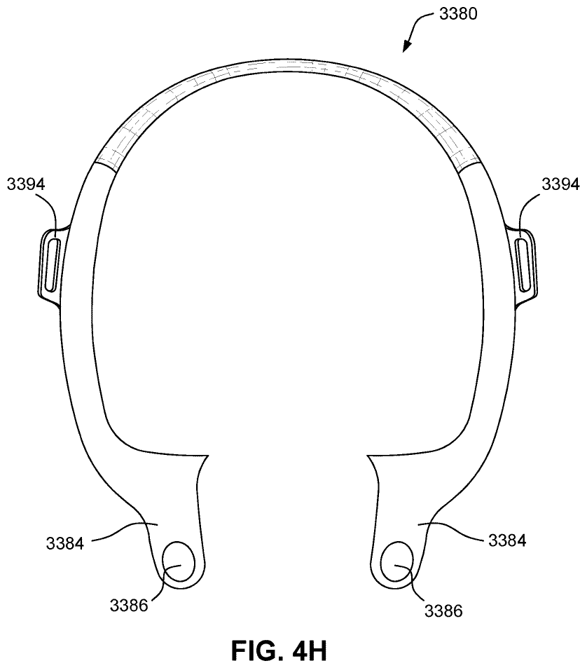

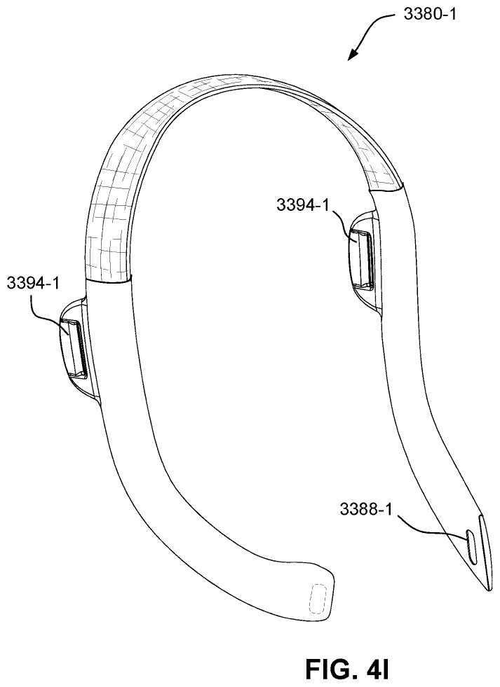

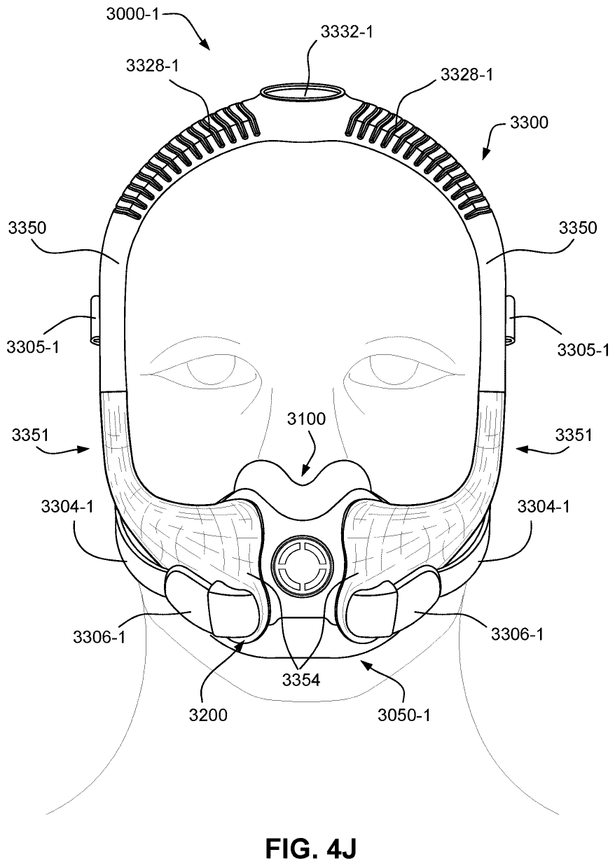

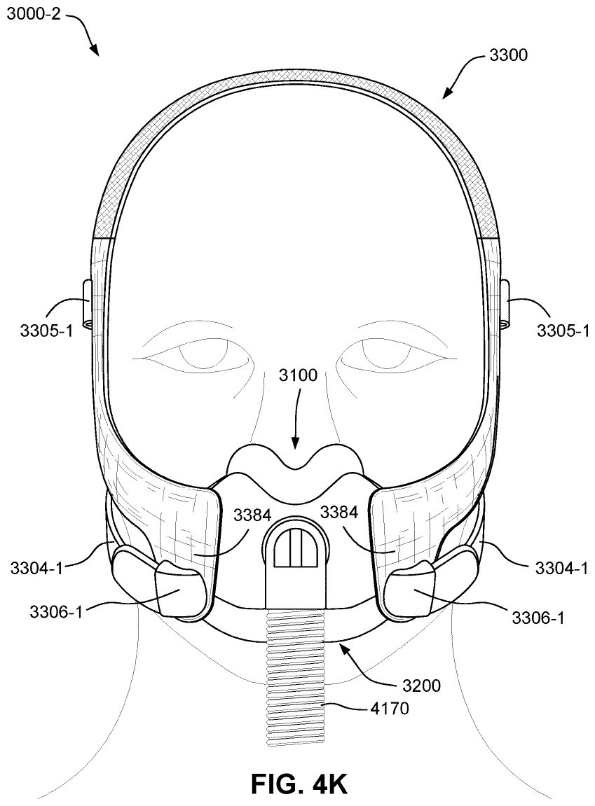

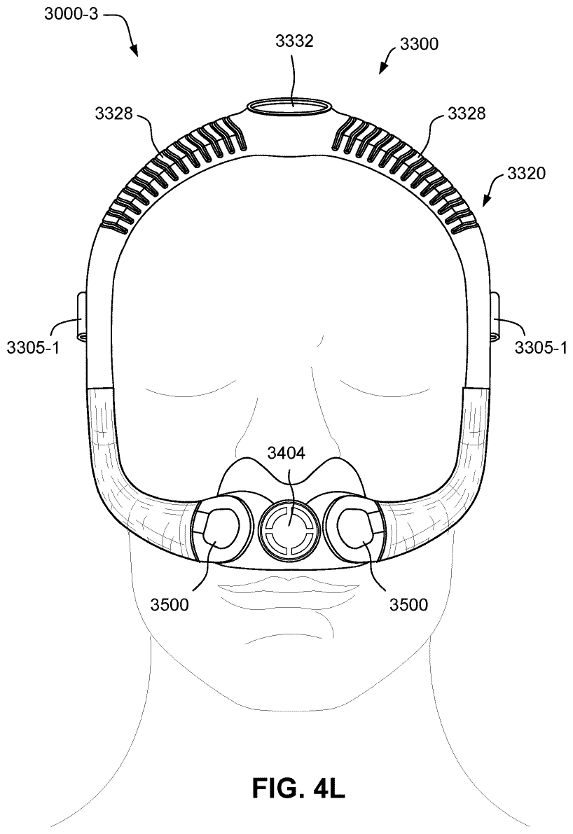

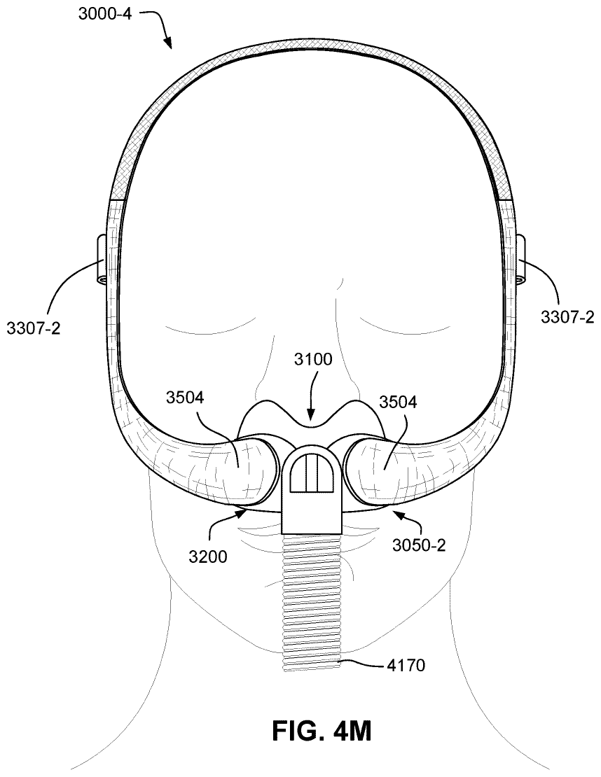

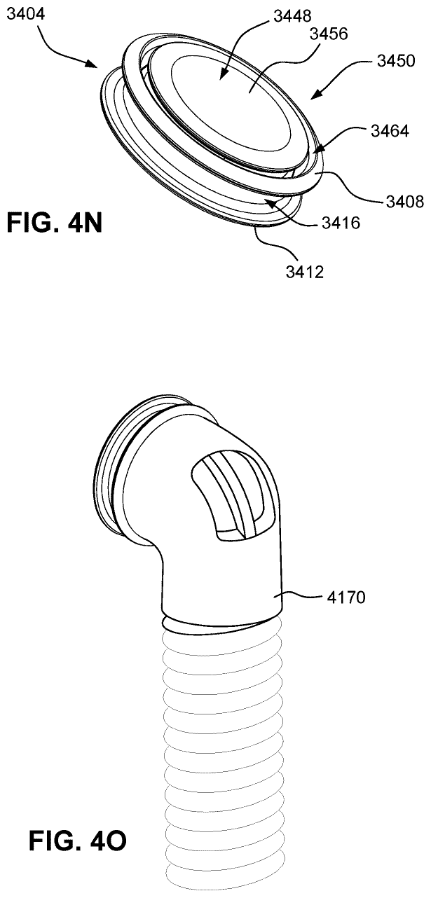

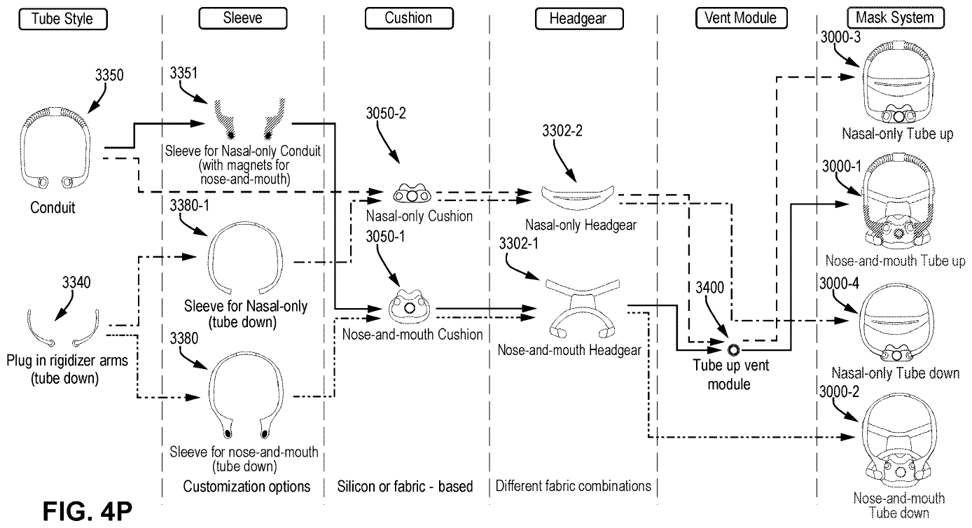

As shown in FIGS. 4A and 4B, different plenum chambers 3200-1, 3200-2 may be formed as part of a multi-opening cushion 3050-1, 3050-2. In the illustrated examples, the cushions 3050-1, 3050-2 each include three openings, although an alternate cushion may be formed with greater or fewer openings.

In some forms, the different openings may serve different functions. For example, some openings may be exclusively inlet openings, while other openings may be exclusively outlet openings.

In other forms, at least one opening may serve two different functions. For example, one opening may operate as both an inlet and an outlet during the same breathing cycle.

The plurality of openings may allow for a variety of configurations of air delivery to the plenum chamber 3200-1, 3200-2. For example, depending on patient need and/or patient comfort, the patient may use a given cushion 3050-1, 3050-2 in a “tube-up” configuration (e.g., using conduit headgear—described below) or a “tube-down” configuration (e.g., using a single conduit in front of the patient's face).

### 5.3.1.1.1 Nose and Mouth Mask

As shown in FIG. 4A, the plenum chamber 3200-1 includes a pair of plenum chamber inlet ports 3254-1, which may be used to convey gas into and/or out of the plenum chamber 3200-1. The plenum chamber inlet ports 3254-1 may be disposed on opposite sides (e.g., left and right sides) of the plenum chamber 3200-1.

In some forms, the plenum chamber 3200-1 may also include at least one vent opening 3402-1 (see e.g., FIG. 4A). The vent opening 3402-1 may be disposed in a center of the plenum chamber 3200-1. For example, the vent opening 3402-1 may be disposed between the plenum chamber inlet ports 3254-1.

In some forms, the plenum chamber 3200-1 may include a pair of grooves 3266-1. Each groove 3266-1 may be disposed proximate to one of the plenum chamber inlet ports 3254-1. Each groove 3266-1 may form a partially recessed surface.

### 5.3.1.1.2 Nose-Only Mask

The plenum chamber 3200-2 of a nasal only cushion 3050-2 may be similar to the plenum chamber 3200-1 of the mouth and nose cushion 3050-1. Only some similarities and differences between the plenum chambers 3200-1, 3200-2 may be described below.

As shown in FIG. 4B, the plenum chamber 3200-2 includes a pair of plenum chamber inlet ports 3254-2, which may be used to convey gas into and/or out of the plenum chamber 3200-2. The plenum chamber inlet ports 3254-2 may be disposed on opposite sides (e.g., left and right sides) of the plenum chamber 3200-2.

In some forms, the plenum chamber 3200-2 may also include at least one vent opening 3402-2 (see e.g., FIG. 4B). The vent opening 3402-2 may be disposed in a center of the plenum chamber 3200-2. For example, the vent opening 3402-2 may be disposed between the plenum chamber inlet ports 3254-2.

In some forms, the plenum chamber 3200-2 may include a pair of grooves 3266-2. Each groove 3266-2 may be disposed proximate to one of the plenum chamber inlet ports 3254-2. Each groove 3266-2 may form a partially recessed surface.

### 5.3.2 Positioning and Stabilising Structure

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g., a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g., resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and, e.g., non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example, the system may comprise one form of positioning and stabilising structure 3300 suitable for a large sized head, but not a small sized head, and another. suitable for a small sized head, but not a large sized head.

### 5.3.2.1 Conduit Headgear

### 5.3.2.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 4J, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 4J, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g., across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 4J, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use. The opening may be an inlet 3332 (see e.g., 4C) for receiving the flow of pressurized air.

In some forms, the third arm of the T-shaped connector may be substantially perpendicular to each of the first two arms.

In some forms, the third arm of the T-shaped connector may be obliquely formed with respect to each of the first two arms.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 3300.

In some forms, at least one of the arms of the T-shaped connector (or Y-shaped connector) may be at least partially rigidised. This may assist in maintaining the shape of the connector so that bending of the connector does not close the airflow path.

The tubes 3350 may be formed from a flexible material, such as an elastomer, e.g., silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

In some examples, the one or more tubes 3350 are crush resistant to resist being blocked if crushed during use, for example if squashed between a patient's head and pillow, especially if there is only one tube 3350. The tubes 3350 may be formed with a sufficient structural stiffness to resist crushing or may be as described in U.S. Pat. No. 6,044,844, the contents of which are incorporated herein by reference.

Each tube 3350 may be configured to receive a flow of air from the connection port 3600 on top of the patient's head and to deliver the flow of air to the seal-forming structure 3100 at the entrance of the patient's airways. In the example shown in FIG. 4J, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g., the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g., a connection port 3600) with respect to a lower portion of the patient interface 3000 (e.g., a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g., an under-the-nose cushion). The positioning and stabilising structure 3300, including the tubes 3350 may be structured and arranged to pull the seal-forming structure 3100 into the patient's face under the nose with a sealing force in a posterior and superior direction (e.g., a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

### 5.3.2.1.2 Extendable and Non-Extendable Tube Portions

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 4J comprises tubes 3350, the superior portions of which comprise extendable tube sections each in the form of an extendable concertina structure 3362.

In some forms, the extendable concertina structure 3328 may be formed as a series of ridges and grooves on the surface of the tubes 3350. The concertina structure 3328 may be biased toward a retracted position, and may move to an expanded position when the patient dons the positioning and stabilising structure 3300. Because portions of the tubes 3350 may be substantially inextensible (e.g., non-extendable tube sections 3363), the concertina structures 3328 permit the positioning and stabilising structure 3300 to stretch in order to fit different sized heads. This may allow a single sized tube 3350 to be used with multiple sized heads. For example, the positioning and stabilising structure 3300 may be “one-size-fits-all” as a result of the concertina structure 3328. Alternatively, the tubes 3350 may be manufactured in multiple sizes (e.g., small, medium, large). The patient may select a length that most closely conforms to their head, and the concertina structures 3328 may make small adjustments in order to tailor the fit to the individual patient.

In some forms, the inlet 3332 may be disposed in the middle of the conduit 6320. For example, the tubes 3350 may be symmetric about the inlet 3332 through at least one axis.

The cross-sectional shape of the non-extendable tube sections 3363 of the tubes 3350 may be circular, elliptical, oval, D-shaped or a rounded rectangle, for example as described in U.S. Pat. No. 6,044,844. A cross-sectional shape that presents a flattened surface of tube on the side that faces and contacts the patient's face or other part of the head may be more comfortable to wear than, for example a tube with a circular cross-section.

In some examples of the present technology, the non-extendable tube sections 3363 connects to the plenum chamber 3200 from a low angle. The headgear tubes 3350 may extend inferiorly down the sides of the patient's head and then curve anteriorly and medially to connect to the plenum chamber 3200 in front of the patient's face. The tubes 3350, before connecting to the plenum chamber 3200, may extend to a location at the same vertical position as (or, in some examples, inferior to) the connection with the plenum chamber 3200. That is, the tubes 3350 may project in an at least partially superior direction before connecting with the plenum chamber 3200. A portion of the tubes 3350 may be located inferior to the plenum chamber 3200 and/or the seal-forming structure 3100. The tubes 3350 may contact the patient's face below the patient's cheekbones, which may be more comfortable than contact on the patient's cheekbones and may avoid excessively obscuring the patient's peripheral vision.

### 5.3.2.1.3 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 4J, the connection port 3600 is located on top of the patient's head (e.g., at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection port 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g., on their back, facing generally upwards) or in a prone position (e.g., on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 5.3.2.1.4 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or a similar sound, so that the patient may easily know that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g., silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 5.3.2.2 Headgear Straps

In some forms, the positioning and stabilising structure 3300 may include headgear 3302 with at least one strap which may be worn by the patient in order to assist in properly orienting the seal-forming structure 3100 against the patient's face (e.g., in order to limit or prevent leaks).

As described above, some forms of the headgear 3302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a sealing force for the seal-forming structure 3100.

Two forms of the headgear, four-point headgear 3302-1 and two-point headgear 3302-2, are discussed in more detail below as illustrative examples.

### 5.3.2.2.1 Four-Point Connection

As shown in FIG. 4E, some forms of the headgear 3302-1 may be a four-point connection headgear. This means that the headgear 3302-1 may connect to four separate places on the plenum chamber 3200, on a frame connected to the plenum chamber 3200, and/or on arms connected to the plenum chamber 3200. The headgear 3302-1 may include four different straps providing a tensile force to help maintain the seal-forming structure 3100 in a sealing position.

In some forms, the headgear 3302-1 may include inferior straps 3304-1, which may connect to an inferior portion of the cushion 3050-1. The inferior straps 3304-1 may extend along the patient's cheek toward a posterior region of the patient's head. For example, the inferior straps 3304-1 may overlay the masseter muscle on either side of the patient's face. The inferior straps 3304-1 may therefore contact the patient's head below the patient's ears. The inferior straps 3304-1 may meet at the posterior of the patient's head, and may overlay the occipital bone and/or the trapezius muscle.

The headgear 3302-1 may also include superior straps 3305-1, which may overlay the temporal bones, parietal bone, and/or occipital bone. The superior straps 3305-1 may also connect to the tubes 3350 (e.g., by interfacing with the tabs 3320).

A rear strap 3307-1 may extend between the superior straps 3305-1 and between the inferior straps 3304-1. The inferior and superior straps 3304-1, 3305-1 on a given side (e.g., left or right) may also be connected to the rear strap 3307-1 adjacent to one another. The height of the rear strap 3307-1 may therefore be approximately the combined height of the inferior and superior strap 3304-1, 3305-1. The rear strap 3307-1 may overlay the occipital bone and/or the parietal bone in use. This may allow the rear strap 3307-1 to assist in anchoring the headgear 3302-1 to the patient's head.

In the illustrated example, the headgear 3302-1 may be formed with a substantially X-shape. The inferior and superior straps 3304-1, 3305-1 may be connected to a rear strap 3307-1 using stitching, ultrasonic welding, or any similar process.

In some forms, the inferior straps 3304-1 are connected to a magnetic member 3306-1. For example, each inferior straps 3304-1 may be threaded through a magnetic member 3306-1, so that a length of each inferior strap 3304-1 may be adjusted. The magnetic members 3306-1 may removably connect to the magnets 3370-1 (described below), so that the inferior straps 3304-1 may be disconnected from the plenum chamber 3200, but the length of the inferior straps 3304-1 may not be affected.

In some forms, the superior straps 3305-1 may be connected directly to the tabs 3320 of the tubes 3350. The superior straps 3305-1 may be threaded through the tabs 3320 in order to adjust the length and control the tensile force of each superior strap 3305-1.

In some forms, the headgear 3302-1 may be used only with the nose and mouth cushion 3050-1 (e.g., because the nose-only cushion 3050-1 does not have four connection points). However, the headgear 3302-1 may be used interchangeably with the tubes 3350 and the rigidiser arms 3340.

### 5.3.2.2.2 Two-Point Connection

As shown in FIG. 4F, some forms of the headgear 3302-2 may be a two-point connection headgear. This means that the headgear 3302-2 may connect to two separate places.

In some forms, the headgear 3302-2 may be formed from a continuous piece of material. In other words, the headgear 3302-2 may not be formed from multiple straps connected (e.g., stitched) together. This may be comfortable for a patient as they will not be in contact with any seams or joints connecting different straps. In other forms, the headgear 3302-2 may be formed from multiple straps (e.g., two superior straps, a rear strap, etc.) that are connected together (e.g., with stitching, ultra-sonic welding, etc.).

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 4F, the patient interface 3000 comprises a rear strap 3307-2 forming part of the positioning and stabilising structure 3300. The rear strap 3307-2 may be known as a back strap or a rear headgear strap, for example. The rear strap 3307-2 may overlay the temporal bones, parietal bone, and/or occipital bone. In other examples of the present technology, one or more further straps may be provided. For example, patient interface 3000 according to examples of the present technology having a nose-and-mouth cushion may have a second, lower, strap configured to lie against the patient's head proximate the patient's neck and/or against posterior surfaces of the patient's neck.

As shown in FIG. 4F, some forms of the headgear 3302-2 may be at least partially bifurcated. For example, a rear strap 3307-2 of the headgear 3302-2 (e.g., configured to contact the posterior portion of the patient's head) may be wider than the surrounding portions of the headgear 3302-2. An intermediate section 3308-2 of the rear strap 3307-2 may include a slit 3309-2. A superior section of the rear strap 3307-2 may therefore be movable relative to the inferior section as a result of the slit 3309-2. This may allow the patient to have a larger strap coverage on the posterior region of their head, which may assist in better anchoring the headgear 3302-2 to the patient's head since there is no inferior strap (e.g., 3304-1).

In some forms, the headgear 3302-2 may be used only with the nasal cushion 3050-2 (e.g., because the nose and mouth cushion 3050-1 does not have four connection points). However, the headgear 3302-2 may be used interchangeably with the tubes 3350 and the rigidiser arms 3340.

### 5.3.2.3 Rigidiser Arm

As shown in FIG. 4D, a rigidiser arm 3340 may be an elongated, rigid member that assists in maintaining the cushion (e.g., the nose and mouth cushion 3050-1 or the nasal cushion 3050-2) in an operating position. The rigidiser arm 3340 may contact a side of the patient's head and provide a force to limit slipping of the seal-forming structure 3100 from the patient's nose and/or mouth.

In some forms, the rigidiser arm 3340 is constructed from a rigid material (e.g., plastic). The rigid material may not permit the rigidiser arm 3340 to stretch.

In some forms, the rigidiser arm 3340 may be flexible along at least one direction. For example, the rigidiser arm 3340 may be flexible about its width and may be inflexible along its length. In other words, the rigidiser arm 3340 may be bendable about an axis along the width of the rigidiser arm 3340, but may be unable to bend about an axis perpendicular to the rigidiser arm 3340. This may allow an individual patient to adjust the rigidiser arm 3340 in order to better fit their individual head.

In certain forms, the rigidiser arm 3340 may remain in the new position after being bent. This may allow a patient adjust the shape of the rigidiser arm 3340 for their specific head and then the rigidiser arm 3340 will keep the desired shape while in use in order to promote patient comfort.

In some forms, a first end 3342 of the rigidiser arm 3340 may be a free end and a second end 3344 (e.g., opposite of the first end 3342) of the rigidiser arm 3340 may be fixed. The first end 3342 may be curved in order to minimize sharp edges that could cause patient discomfort. The first end 3342 may also overlay the patient's head proximate to the temporal bone, in use. The second end 3344 may be fixed to an arm connection structure 3504.

In some forms, the arm connection structure 3504 may be similar to the conduit connection structure 3500. For example, the arm connection structure 3504 and the conduit connection structure 3500 may have substantially the same shape. This may allow either the conduit connection structure 3500 or the arm connection structure 3504 to fit into the groove (e.g., 3266-1 or 3266-2) and connect to the plenum chamber inlet port 3254. The arm connection structure 3504 may connect to the nose and mouth cushion 3050-1 or the nose-only cushion 3050-2 in substantially the same way as the conduit connection structure 3500 (e.g., via a snap fit, press fit, friction fit, etc.).

In some forms, the arm connection structure 3504 may act as a plug for the plenum chamber inlet port 3254 (e.g., either 3254-1 and/or 3254-2). Unlike the tubes 3350, the rigidiser arm 3340 does not convey pressurized air to the plenum chamber 3200. The rigidiser arm 3340 may be used with a “tube down” configuration, where a hose is connected to the vent opening 3402 (e.g., either 3402-1 and/or 3402-2), and conveys air into the plenum chamber 3200 through the vent opening 3402. In this example, air does not need to travel into or out of the plenum chamber inlet ports 3254. Thus, the arm connection structure 3504 may form a seal with the plenum chamber inlet port 3254 in order to limit airflow into or out of the plenum chamber 3200.

### 5.3.3 Vent

In one form, the patient interface 3000 includes a vent 3400 constructed and arranged to allow for the washout of exhaled gases, e.g., carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

As shown in FIG. 4N, a vent 3450 may be used with the patient interface 3000. The vent 3450 may have a substantially similar shape to the vent opening 3402-1 (e.g., a substantially circular shape).

The vent 3450 may be used with either the mouth and nose plenum chamber 3200-1 (e.g., illustrated in FIG. 4A) or the nose-only plenum chamber 3200-2 (e.g., illustrated in FIG. 4B).

With continued reference to FIG. 4A, the vent 3450 may include a vent housing 3404, which may be configured to engage with the vent opening 3402. The vent housing 3404 may be constructed from a rigid material or a semi-rigid material. For example, the vent housing 3404 may be constructed from plastic, metal, or any similar material. The vent housing 3404 may add rigidity to the patient interface 3000 (e.g., to limit unwanted bending that may affect the position of the seal-forming structure 3100 on the patient's face).

The vent housing 3404 may include an anterior surface 3408, a posterior surface 3412, and a groove 3416. The anterior surface 3408 faces away from the patient's face in use, and may be positioned outside the pressurized volume of the plenum chamber 3200. The posterior surface 3412 is disposed opposite to the anterior surface 3408. In use, the posterior surface 3412 may face the patient and may be disposed within the pressurized volume of the plenum chamber 3200. The groove 3416 may be formed between the anterior and posterior surfaces 3408, 3412. A portion of the plenum chamber 3200 may be received within the groove 3416 in order to retain the vent 3400 in position.

In some forms, a diffuser 3448 may be used with the vent housing 3404. The diffuser 3448 may assist with limiting the decibel output from any of the patient interface 3000 (or any other patient interface). Specifically, the diffuser 3448 may assist in limiting the decibel level associated with air output from the patient interface 3000 (e.g., exhaled air), although the diffuser 3448 may limit the decibel level of at any point in the patient interface.

In certain forms, the diffuser 3448 may diffuse, and therefore slow, the exhaust gas exiting the plenum chamber 3200 and passing through the vent housing 3404. The diffuser 3448 may assist in avoiding jetting and associated discomfort to the patient and/or bed partner (e.g., noise caused by jetting against a pillow, sheets, bedclothes, etc.).

In some forms, the diffuser may include an anterior surface 3456 that faces away from the patient in use. An outer diameter of the anterior surface 3456 may be less than an inner diameter of the vent housing 3404 proximate to the anterior surface 3408. This may form a gap 3464 through which air may travel.

### 5.3.4 Decoupling Structure(s)

In one form the patient interface 3000 includes at least one decoupling structure, for example, a swivel or a ball and socket.

### 5.3.5 Modularity

As described above, the cushion, headgear, and sleeves may come in different styles, which may correspond to different uses (e.g., mouth breathing, nasal breathing, etc.). A patient or clinician may select certain combinations of cushions, headgear, and sleeves in order to optimize the effectiveness of the therapy and/or the individual patient's comfort. An example of this sort of modular design is described in PCT/SG2022/050777 filed 28 Oct. 2022, incorporated herein by reference in its entirety.

In some forms, the different styles of cushions, headgear, and sleeves may be used interchangeably with one another in order to form different combinations of patient interfaces. This may be beneficial from a manufacturing perspective because wider variety of patient interfaces may be created using fewer parts. Additionally or alternatively, the various combinations may allow a patient to change styles of patient interface without changing every component.

Air may be delivered to the patient in one of two main ways. In one example, the patient may receive the flow of pressurized air through headgear tubes 3350 (see, e.g., FIG. 4C, 4J). This may be referred to as a “tube up” configuration and may position a connection port at the top of the patient's head. In other example, the patient may receive the flow of pressurized air through a conduit connected to the plenum chamber 3200. This may be referred to a “tube down” configuration where the airflow conduit is positioned in front of the patient's face. Different patients may be more comfortable with one style of air delivery over the other (e.g., because of the patient's sleep style). Therefore, it may be beneficial to allow a single style of patient interface to be used in either the “tube up” or “tube down” configuration.

The patient interface may be part of a modular assembly with a variety of interchangeable components that may be swapped out by a patient and/or clinician for one or more components for a different style. The following description describes the various combinations that may be created by assembling the different components together.

### 5.3.5.1 Sleeve

In some forms, to allow for modularity, a sleeve may be used with the tubes 3350 and/or the rigidiser arms 3340. The sleeve may at least partially surround the tubes 3350 and/or the rigidiser arms 3340. As shown in FIGS. 4G to 4I, different shapes of sleeves may be used, which may correspond to different types of positioning and stabilising structures 3300. In some forms, the configuration of the sleeve may be customized to fit a particular user's face. For instance, the sleeves may be configured in a relatively more posterior region of the patient's head.

In some forms, the sleeve may be constructed from a comfortable material. For example, the sleeve may be constructed from a textile material, a foam material, or a combination of the two. The comfortable material may contact the patient in use, and may feel soft against the patient's skin in order to improve patient compliance.

The material may also be flexible in order to assist in donning or doffing the sleeve from the tube 3350 or the rigidiser arms 3340. For example, the material may allow the sleeve to bend in order to conform to the shape of the tubes 3350 or conduit headgear or the rigidiser arms 3340, which may change depending on the shape of an individual patient's head.

In some forms, the sleeve may also be at least partially elastic (e.g., the material may allow the sleeve to stretch). The elastic material may help the sleeve stretch in order to fit around the tubes 3350 or the rigidiser arms 3340. The elastic material may then return to an initial position that is snug against the tubes 3350 or the rigidiser arms 3340 in order to limit the sleeve from sliding while in use.

As described in more detail below, some forms of the sleeves may be specific to a rigidising element (e.g., tubes 3350 and/or rigidiser arms 3340). However, the sleeves may assist the rigidising elements in connecting interchangeably with the version or styles of cushions (e.g., the mouth and nose cushion 3050-1, the nose-only cushion 3050-2, etc.).

### 5.3.5.2 Conduit Sleeve

As shown in FIG. 4G, one example of a sleeve is a conduit sleeve 3351, which may be usable with the tubes 3350 described above.

As shown in FIG. 4G, the conduit sleeve 3351 may include a curved shape that may be similar to the shape of the tubes 3350 shown in FIG. 4C. The flexible material used to construct the conduit sleeve 3351 may allow the conduit sleeve 3351 to further curve in order to correspond to the shape of the tubes 3350 (e.g., when worn by the patient).

In some forms, the conduit sleeve 3351 may include a first or superior opening 3352. The superior opening 3352 may be disposed at one end of the conduit sleeve 3351. The superior opening 3352 may be an opening to a passage that extends along at least a portion of the conduit sleeve 3351.

As shown in FIG. 4G, some forms of the conduit sleeve 3351 may also include an inferior extension 3354. The inferior extension 3354 may be positioned on an opposite end of the conduit sleeve 3351 from the superior opening 3352. The conduit sleeve 3351 may be customized to fit a particular user's face. For instance, the inferior extension 3354 of the conduit sleeve 3351 may be configured in a relatively more posterior region or anterior region of the patient's head.

Some forms of the inferior extension 3354 may include a rigid or semi-rigid piece (e.g., within the conduit sleeve 3351). The rigid or semi-rigid piece may be constructed from a plastic material, or a similar material. Alternatively, the inferior extension 3354 may be stiffened using a manufacturing process (e.g., stitching rigidised thread, flat knitting, using thicker material).

As shown in FIG. 4G, some forms of the inferior extension 3354 may include a connection member 3356. In the illustrated example, the connection member 3356 may be a magnet, although in other examples, the connection member 3356 may be a different type of connector (e.g., a mechanical fastener, an adhesive, hook and loop material, etc.). The connection member 3356 may also be positioned at an end of the inferior extension 3354, although the connection member 3356 could alternatively be positioned anywhere along the inferior extension 3354.

In some forms, the connection member 3356 (e.g., a magnet) may be removably connected to the magnets 3370-1 of the headgear 3302-1. For example, when the conduit sleeves 3351 are connected to the tubes 3350 (see e.g., FIG. 4J), the magnets 3370-1 connected to the inferior straps 3304-1 may be removably connected to the connection member 3356 in order to provide the tensile force.

### 5.3.5.2.1 Four-Point Arm Sleeve

As shown in FIG. 4H, Another Example of a Sleeve is a Four-Point Arm Sleeve 3380, which may be usable with the rigidiser arms 3340 described above.

As shown in FIG. 4H, the four-point arm sleeve 3380 may include a curved shape that may be similar to the shape of the rigidiser arm 3340 shown in FIG. 4D. The flexible material used to construct the four-point arm sleeve 3380 may allow the four-point arm sleeve 3380 to further curve in order to correspond to the shape of the rigidiser arm 3340 (e.g., when worn by the patient and/or went bent by the patient).

As shown in FIG. 4H, some forms of the four-point arm sleeve 3380 may include an inferior extension 3384. The inferior extension 3384 may be positioned at an end of the four-point arm sleeve 3380.

In the illustrated example, the shape and/or structure of the inferior extension 3384 is substantially the same as the shape of the inferior extension 3354. For example, the inferior extension 3384 may be more rigid as compared to the rest of the four-point arm sleeve 3380 (e.g., as a result of rigidising thread or rigid material).

As shown in FIG. 4H, some forms of the inferior extension 3384 may include a connection member 3386. In the illustrated example, the connection member 3386 may be a magnet, although in other examples, the connection member 3386 may be a different type of connector (e.g., a mechanical fastener, an adhesive, hook and loop material, etc.). The connection member 3386 may also be positioned at an end of the inferior extension 3384, although the connection member 3386 could alternatively be positioned anywhere along the inferior extension 3384.

In some forms, the connection member 3386 (e.g., a magnet) may be removably connected to the magnets 3370-1 of the headgear 3302-1. For example, when the four-point arm sleeves 3380 are connected to the rigidiser arm 3340 (see e.g., FIG. 4K), the magnets 3370-1 connected to the inferior straps 3304-1 may be removably connected to the connection member 3386 in order to provide the tensile force.

As shown in FIG. 4H, the four-point arm sleeve 3380 may include a pair of tabs 3394, which may be similar to the tab 3320 on the tubes 3350. When the four-point arm sleeve 3380 is worn by the patient, the tabs 3394 may be positioned in substantially the same place on the patient's head as where the tabs 3320 are positioned when the patient wears the tubes 3350.

### 5.3.5.2.2 Two-Point Arm Sleeve

As shown in FIG. 4I, yet another example of a sleeve is a two-point arm sleeve 3380-1, which may be usable with the rigidiser arms 3340 described above.

In some forms, the two-point arm sleeve 3380-1 may be similar to the four-point arm sleeve 3380 described above. Only some similarities and differences may be described below.

As shown in FIG. 4I, the two-point arm sleeve 3380-1 may include an inferior opening 3388-1 that is positioned at an end of the two-point arm sleeve 3380-1. The inferior opening 3388-1 may form an opening to a passageway through the two-point arm sleeve 3380-1. In the illustrated example, the inferior opening 3388-1 may open into a surface of the conduit sleeve 3380-1.

As shown in FIG. 4I, the two-point arm sleeve 3380-1 may include a pair of tabs 3394-1, which may be similar to the tab 3320 on the tubes 3350. When the two-point arm sleeve 3380-1 is worn by the patient, the tabs 3394-1 may be positioned in substantially the same place on the patient's head as where the tabs 3320 are positioned when the patient wears the tubes 3350.

### 5.3.5.3 Assembled Patient Interfaces

As illustrated in FIGS. 4J to 4M, the various elements described above may be combined into four different patient interfaces. The different patient interfaces may allow patients to use different styles based on their individual comfort. The modularity of the different elements (e.g., the ability to be used in multiple styles of patient interfaces) may simplify manufacturing and/or may allow a patient to more easily switch between styles of patient interfaces.

### 5.3.5.3.1 Nose and Mouth Mask Tube Up Configuration

As illustrated in FIG. 4J, the patient may wear the cushion 3050-1 in a tube-up configuration with the tubes 3350 and the four-point headgear 3302-1. This assembly may form a tube up nose and mouth patient interface 3000-1.

In some forms, a conduit sleeve may be used with the tubes 3350 in order to enable a patient to experience the “tube up” air delivery style with the mouth and nose cushion 3050-1. As is described below, the conduit sleeve provides additional connection locations for connecting the four-point headgear 3302-1. However, other forms of connectors aside from or in addition to the conduit sleeve may be used.

In the illustrated example, the conduit sleeves may be connected to the tubes 3350 of the positioning and stabilising structure 3300. The tubes 3350 (via the conduit connection structure 3500), may be used to connect the tubes 3350 to the cushion 3050-1. The conduit sleeves provide the magnets in order to connect to the magnets 3370-1 (see e.g., FIG. 4E) of the four-point headgear 3302-1. Alternatively, a different connection form may be used.

As illustrated in FIG. 4J, the four-point headgear 3302-1 may connect in four separate locations in order to provide a tensile force that maintains the cushion 3050-1 in a sealing position on the patient's head.

For example, the inferior straps 3304-1 (e.g., via the magnetic members 3306-1) may removably connect to the magnets of the conduit sleeves. In use, each inferior strap 3304-1 may contact the patient's cheek (e.g., overlaying the masseter muscle). The inferior straps 3304-1 may also extend below the patient's ears.

### 5.3.5.3.2 Nose and Mouth Mask Tube Down Configuration

As illustrated in FIG. 4K, the patient may wear the cushion 3050-1 in a tube-down configuration with the rigidiser arms 3340 and the four-point headgear 3302-1. This assembly may form a tube down nose and mouth patient interface 3000-2.

In some forms, a conduit sleeve may be used with the rigidiser arms 3340 in order to enable a patient to experience the “tube down” air delivery style with the mouth and nose cushion 3050-1. As is described below, the conduit sleeve provides additional connection locations for connecting the four-point headgear 3302-1. However, other forms of connectors aside from or in addition to the conduit sleeve may be used.

In the illustrated example, the conduit sleeves may be connected to the rigidiser arms 3340 of the positioning and stabilising structure 3300. The rigidiser arms 3340 (via the conduit connection structure 3504), may be used to connect the rigidiser arms 3340 to the cushion 3050-1. The conduit sleeves provide the magnets in order to connect to the magnets 3370-1 (see e.g., FIG. 4E) of the four-point headgear 3302-1. Alternatively, a different connection form may be used.

As illustrated in FIG. 4K, the four-point headgear 3302-1 may connect in four separate locations in order to provide a tensile force that maintains the cushion 3050-1 in a sealing position on the patient's head.

For example, the inferior straps 3304-1 (e.g., via the magnetic members 3306-1) may removably connect to the magnets of the conduit sleeves. In use, each inferior strap 3304-1 may contact the patient's cheek (e.g., overlaying the masseter muscle). The inferior straps 3304-1 may also extend below the patient's ears.

### 5.3.5.3.3 Nose Mask Tube Up Configuration

As illustrated in FIG. 4L, the patient may wear the cushion 3050-2 in a tube-up configuration with the tubes 3350 and the two-point headgear 3302-2. This assembly may form a tube up nose only patient interface 3000-3

A conduit sleeve may be used with the tubes 3350, and may provide additional comfort to the patient. The sleeve may not add additional connection points to connect the positioning and stabilising structure 3300 on the cushion 3050-2. In the illustrated example, the tubes 3350 of the positioning and stabilising structure 3300 may be connected directly to the cushion 3050-2.

As illustrated in FIG. 4L, the two-point headgear 3302-2 may connect to the tabs 3320 on the tubes 3350 in order to provide a tensile force that maintains the cushion 3050-2 in a sealing position on the patient's head.

### 5.3.5.3.4 Nose Mask Tube Down Configuration

As illustrated in FIG. 4M, the patient may wear the cushion 3050-2 in a tube-up configuration with the rigidiser arms 3340 and the two-point headgear 3302-2. This assembly may form a tube down nose only patient interface 3000-4.

A conduit sleeve may be used with the rigidiser arms 3340, and may provide additional comfort to the patient. The sleeve may not add additional connection points to connect the positioning and stabilising structure 3300 on the cushion 3050-2. In the illustrated example, the rigidiser arms 3340 of the positioning and stabilising structure 3300 may be connected directly to the cushion 3050-2.

As illustrated in FIG. 4M, the two-point headgear 3302-2 may connect to the tabs 3320 on the sleeve in order to provide a tensile force that maintains the cushion 3050-2 in a sealing position on the patient's head.

### 5.3.5.3.5 Modularity of Elements

FIG. 4P illustrates how the different elements can be combined in order to form the four different patient interfaces described above. As illustrated, the different components may be reused for different styles of patient interfaces. This may allow for easier manufacturing and assembly, because a large number of the same components may be produced and used in a variety of styles. The only components not used in multiple styles may be the sleeves. However, the sleeves may be easier to manufacture. FIG. 4O shows a portion of air circuit 4170 that may interface with the patient interface, while FIG. 4N shows a vent housing 3404 that may interchangeably replace the air circuit shown in FIG. 4O, depending on the style of the patient interface.

### 5.4 RPT Device

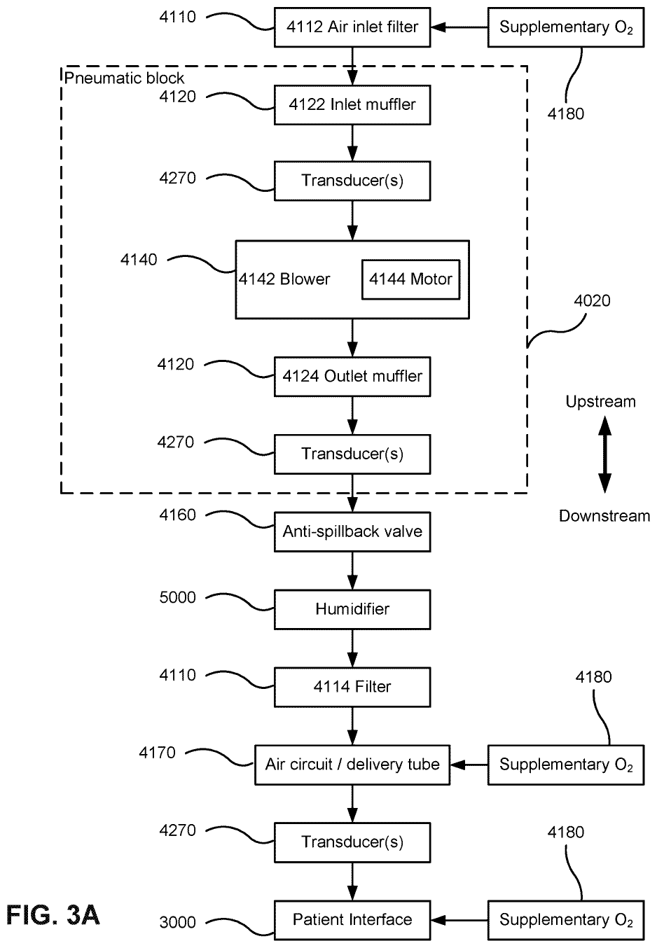

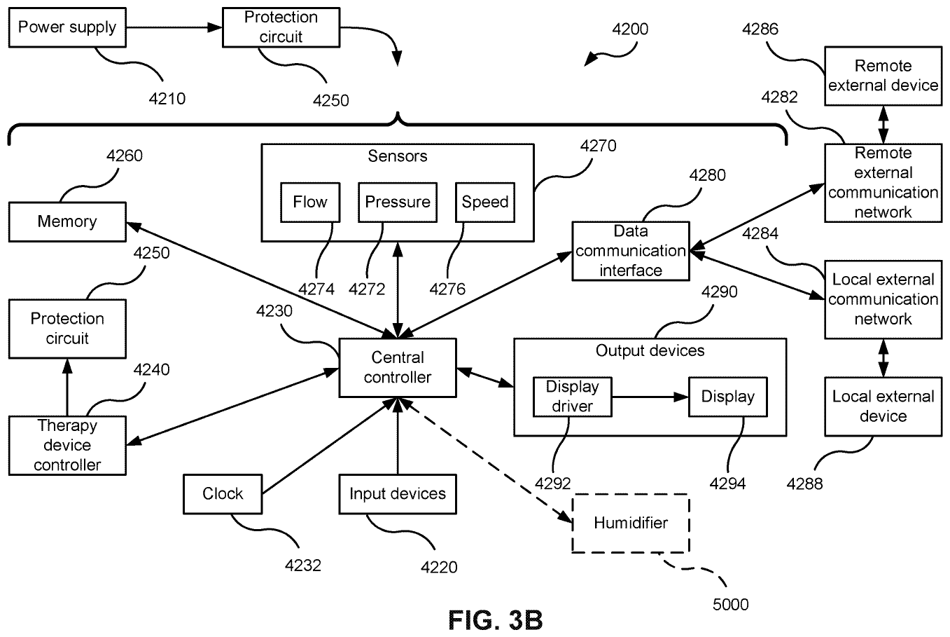

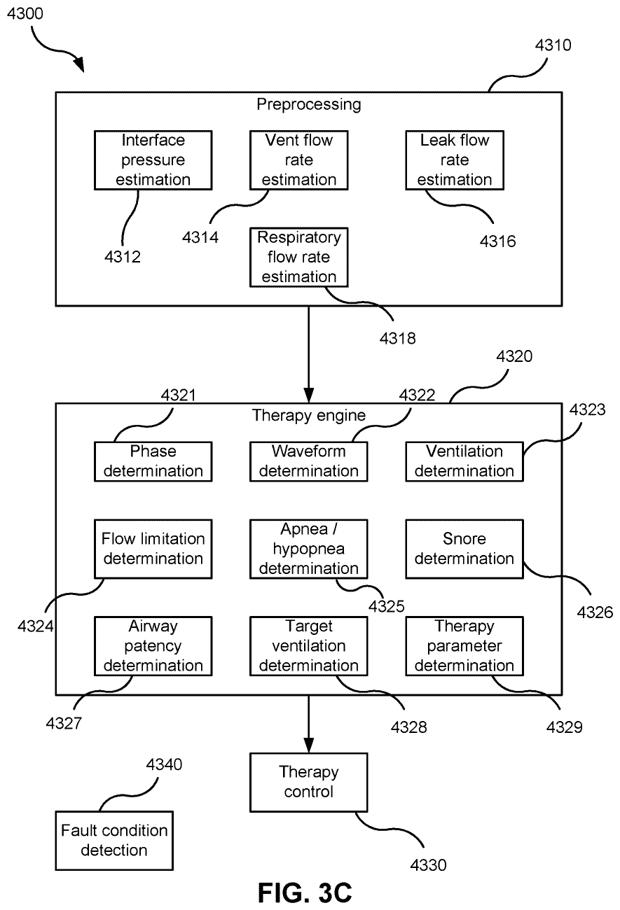

As shown in FIGS. 3A-3C, an RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 5.4.1 Air Filter(s)

An RPT device in accordance with one form of the present technology may include an air filter 4110, or a plurality of air filters 4110.

In one form illustrated in FIG. 3A, an inlet air filter 4112 is located at the beginning of the pneumatic path upstream of a pressure generator 4140.

In one form illustrated in FIG. 3A, an outlet air filter 4114, for example an antibacterial filter, is located between an outlet of the pneumatic block 4020 and a patient interface 3000 or 3800.

### 5.4.2 Muffler(s)

An RPT device in accordance with one form of the present technology may include a muffler 4120, or a plurality of mufflers 4120.

In one form of the present technology (see e.g., FIG. 3A), an inlet muffler 4122 is located in the pneumatic path upstream of a pressure generator 4140.

In one form of the present technology, an outlet muffler 4124 is located in the pneumatic path between the pressure generator 4140 and a patient interface 3000 or 3800.

### 5.4.3 Pressure Generator

In one form of the present technology, a pressure generator 4140 for producing a flow, or a supply, of air at positive pressure is a controllable blower 4142.

The pressure generator 4140 may be under the control of the therapy device controller 4240.

In other forms, a pressure generator 4140 may be a piston-driven pump, a pressure regulator connected to a high pressure source (e.g., compressed air reservoir), or a bellows.

### 5.4.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology (see e.g., FIG. 3A), one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800.

In one form, a signal from a transducer 4270 may be filtered, such as by low-pass, high-pass or band-pass filtering.

### 5.4.5 RPT Sensors

### 5.4.5.1 Flow Rate Sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 5.4.5.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 and representing a pressure is received by the central controller 4230.

### 5.4.6 Motor Speed Transducer

In one form of the present technology a motor speed transducer 4276 is used to determine a rotational velocity of a motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.4.7 Anti-Spill Back Valve

As shown in FIG. 3A, one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.4.8 RPT Device Electrical Components

### 5.4.8.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.4.8.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.4.8.3 Central Controller

In one form of the present technology, the central controller 4230 Is One or a plurality of processors suitable to control an RPT device 4000. The central controller 4230 is shown in FIG. 3B.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and/or the humidifier 5000.

The central controller 4230 may be configured to provide output signal(s) to one or more of an output device 4290, a pressure generator 4140, a therapy device controller 4240, a data communication interface 4280, and/or the humidifier 5000.

In some forms of the present technology, the central controller 4230 is configured to implement the one or more methodologies described herein, such as the one or more algorithms 4300 which may be implemented with processor-control instructions, expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.8.4 Clock

The RPT device 4000 may include a clock 4232 that is connected to the central controller 4230.

### 5.4.8.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 5.4.8.6 Protection Circuits

The one or more protection circuits 4250 in accordance with the present technology may comprise an electrical protection circuit, a temperature and/or pressure safety circuit.

### 5.4.8.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.8.8 Data Communication Systems

In one form of the present technology, a data communication interface 4280 is provided, and is connected to the central controller 4230 (see e.g., FIG. 3B). Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 5.4.8.9 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.4.8.9.1 Display Driver

A display driver 4292 receives as an input the characters, symbols, or images intended for display on the display 4294, and converts them to commands that cause the display 4294 to display those characters, symbols, or images.

### 5.4.8.9.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

Referring now to FIGS. 3A-3C, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.4.8.10 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the vent flow rate Qv, the respiratory flow rate Qr, and the leak flow rate Ql.

In various forms of the present technology, the pre-processing module 4310 comprises one or more of the following algorithms: interface pressure estimation algorithm 4312, vent flow rate estimation algorithm 4314, leak flow rate estimation algorithm 4316, and respiratory flow rate estimation algorithm 4318.

### 5.4.8.10.1 Interface Pressure Estimation

In one form of the present technology, an interface pressure estimation algorithm 4312 receives as inputs a signal from the pressure sensor 4272 indicative of the pressure in the pneumatic path proximal to an outlet of the pneumatic block (the device pressure Pd) and a signal from the flow rate sensor 4274 representative of the flow rate of the airflow leaving the RPT device 4000 (the device flow rate Qd). The device flow rate Qd, absent any supplementary gas 4180, may be used as the total flow rate Qt. The interface pressure estimation algorithm 4312 estimates the pressure drop ΔP through the air circuit 4170. The dependence of the pressure drop ΔP on the total flow rate Qt may be modelled for the particular air circuit 4170 by a pressure drop characteristic ΔP(Q). The interface pressure estimation algorithm, 4312 then provides as an output an estimated pressure, Pm, in the patient interface 3000 or 3800. The pressure, Pm, in the patient interface 3000 or 3800 may be estimated as the device pressure Pd minus the air circuit pressure drop ΔP.

### 5.4.8.10.2 Vent Flow Rate Estimation

In one form of the present technology, a vent flow rate estimation algorithm 4314 receives as an input an estimated pressure, Pm, in the patient interface 3000 or 3800 from the interface pressure estimation algorithm 4312 and estimates a vent flow rate of air, Qv, from a vent 3400 in a patient interface 3000 or 3800. The dependence of the vent flow rate Qv on the interface pressure Pm for the particular vent 3400 in use may be modelled by a vent characteristic Qv(Pm).

### 5.4.8.10.3 Leak Flow Rate Estimation

In one form of the present technology, a leak flow rate estimation algorithm 4316 receives as an input a total flow rate, Qt, and a vent flow rate Qv, and provides as an output an estimate of the leak flow rate Ql. In one form, the leak flow rate estimation algorithm estimates the leak flow rate Ql by calculating an average of the difference between total flow rate Qt and vent flow rate Qv over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface 3000 or 3800, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 5.4.8.10.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

### 5.4.8.11 Therapy Engine Module

In one form of the present technology, a therapy engine module 4320 Receives as inputs one or more of a pressure, Pm, in a patient interface 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 5.4.8.11.1 Phase Determination

In one form of the present technology, the RPT device 4000 does not determine phase.

In one form of the present technology, a phase determination algorithm 4321 receives as an input a signal indicative of respiratory flow rate, Qr, and provides as an output a phase Φ of a current breathing cycle of a patient 1000.

### 5.4.8.11.2 Waveform Determination

In one form of the present technology, the therapy parameter determination algorithm 4329 provides an approximately constant treatment pressure throughout a respiratory cycle of a patient.

In other forms of the present technology, the therapy control module 4330 controls the pressure generator 4140 to provide a treatment pressure Pt that varies as a function of phase Φ of a respiratory cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

### 5.4.8.11.3 Ventilation Determination

In one form of the present technology, a ventilation determination algorithm 4323 receives an input a respiratory flow rate Qr, and determines a measure indicative of current patient ventilation, Vent.

### 5.4.8.11.4 Determination of Inspiratory Flow Limitation

In one form of the present technology, the central controller 4230 executes an inspiratory flow limitation determination algorithm 4324 for the determination of the extent of inspiratory flow limitation.

### 5.4.8.11.5 Determination of Apneas and Hypopneas

In one form of the present technology, the central controller 4230 executes an apnea/hypopnea determination algorithm 4325 for the determination of the presence of apneas and/or hypopneas.

### 5.4.8.11.6 Determination of Snore

In one form of the present technology, the central controller 4230 executes one or more snore determination algorithms 4326 for the determination of the extent of snore.

### 5.4.8.11.7 Determination of Airway Patency

In one form of the present technology, the central controller 4230 executes one or more airway patency determination algorithms 4327 for the determination of the extent of airway patency.

### 5.4.8.11.8 Determination of Target Ventilation

In one form of the present technology, the central controller 4230 takes as input the measure of current ventilation, Vent, and executes one or more target ventilation determination algorithms 4328 for the determination of a target value Vtgt for the measure of ventilation.

### 5.4.8.11.9 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

### 5.4.8.12 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 or 3800 is equal to the treatment pressure Pt.

### 5.4.8.13 Detection of Fault Conditions

In one form of the present technology, the central controller 4230 executes one or more methods 4340 for the detection of fault conditions. The fault conditions detected by the one or more methods 4340 may include at least one of the following:
Power failure (no power, or insufficient power)
Transducer fault detection
Failure to detect the presence of a component
Operating parameters outside recommended ranges (e.g. pressure, flow rate, temperature, PaO2)
Failure of a test alarm to generate a detectable alarm signal.

Upon detection of the fault condition, the corresponding algorithm signals the presence of the fault by one or more of the following:
Initiation of an audible, visual &/or kinetic (e.g. vibrating) alarm
Sending a message to an external device
Logging of the incident

### 5.5 Patient Interface Detection

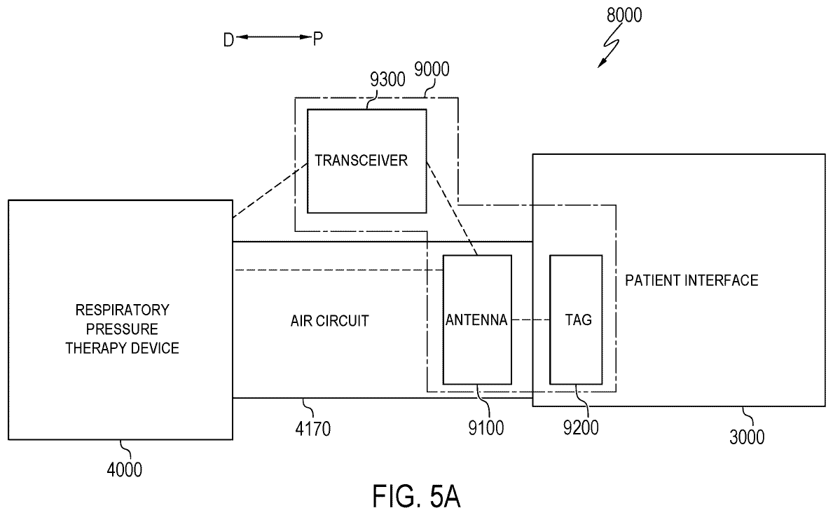

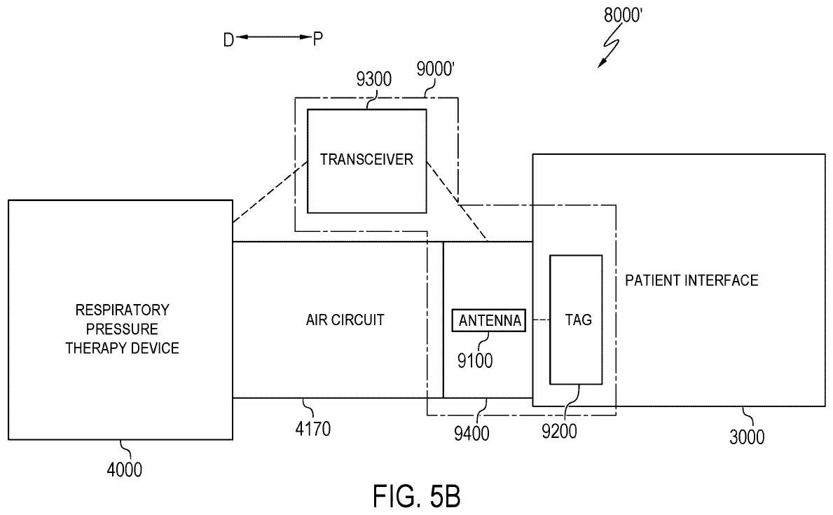

FIG. 5A illustrates a schematic view of a respiratory therapy system 8000. The Respiratory therapy system 8000 may be configured to wirelessly detect information about the patient interface 3000 or an accessory that a patient is using during a therapy session. In some aspects, incorporation of the technology described further below may create a patient interface (or accessory) detection system in which a patient does not need to manually scan in or input (e.g., via an external device like a smartphone, computer, or tablet, or via mechanism within the respiratory therapy system) the type of patient interface being used. In other aspects, incorporation of a wireless patient interface detection system may allow the respiratory therapy system 8000 to confirm the accuracy of a patient's input regarding the type of patient interface (or accessory) being used, or may track usage or other characteristics of the patient interface (or accessory) in use, as described further below.

The respiratory therapy system 8000 includes the patient interface 3000 and the respiratory pressure therapy (RPT) device 4000 fluidly coupled by the air circuit 4170, which may be a conduit or tube, as described above. As further described above, the RPT device 4000 is configured to supply a flow of gas, for example, air, which may be supplemented with oxygen, through the air circuit 4170 to the patient interface 3000. The RPT device 4000 may include any of the elements described above, such as a humidifier, an oxygen source, and/or data management systems.

The RPT device 4000 may be used individually or as part of the system 8000 to deliver one or more of a number of therapies described above, such as by operating the device to generate a flow of air for delivery to an interface (e.g., the patient interface 3000) to the airways of the user. The flow of air may be pressure-controlled (for respiratory pressure therapies) or flow-controlled (for flow therapies such as HFT). Thus the RPT device 4000 may also act as a flow therapy device. The RPT device 4000 may, for example, include a CPAP device and/or a ventilator.

The respiratory therapy system 8000 may further comprise a Radio Frequency Identification (RFID) system 9000. The RFID system 9000 may, for example, be configured to detect one or more characteristics of the respiratory therapy being delivered from the RPT device 4000 to the patient interface 3000, the identification of the patient interface 3000, and/or the identification of an accessory device (not shown) directly or indirectly coupled to the respiratory therapy system 8000. Exemplary accessory devices may include, but are not limited to, a patient interface headgear, a cushion on the patient interface 3000, an air filter, a humidifier, one or more components of a humidification system (e.g., components of a heat and moisture exchanger or waterless humidifier), a conduit, and/or an adapter accessory.

RFID is a form of wireless communication that incorporates the use of electromagnetic or electrostatic coupling in the radio frequency portion of the electromagnetic spectrum, for example, to uniquely identify and/or track an object. The RFID system 9000 may operate according to the principle of inductive coupling.

The RFID system 9000 includes a transponder 9200 (hereinafter referred to as a “tag”), a transceiver 9300, and an antenna 9100. In some embodiments, the RFID system 9000 includes one antenna 9100. In alternative embodiments, to be described in further detail herein, the RFID system 9000 includes two or more antennas 9100. The antenna 9100 is configured to emit radio waves and receive reflected signals, for example, from the tag 9200. The antenna 9100 may be a linear antenna, and thus may emit a linear polarized signal, or the antenna 9100 may be a circular antenna, and thus may emit a circularly polarized signal. In some embodiments, the RFID system 9000 may include both linear antenna(s) and circular antenna(s).

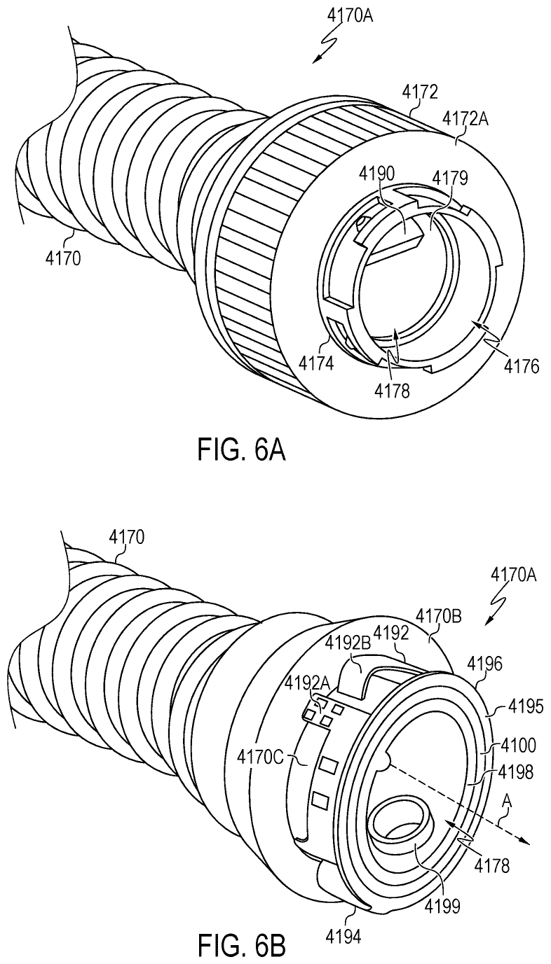

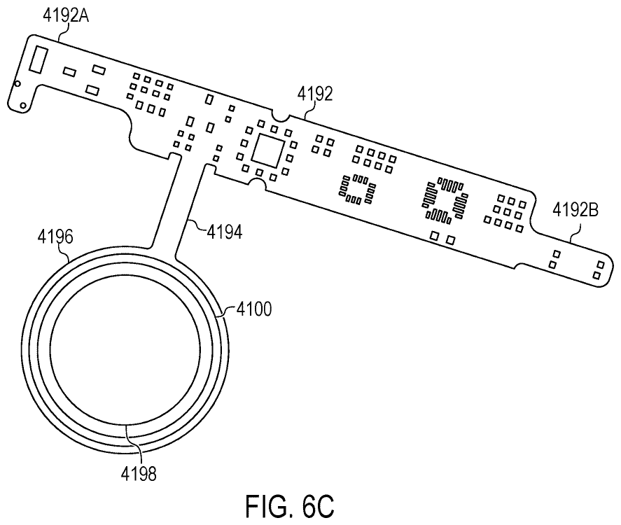

The antenna 9100 may be disposed within a proximal portion (e.g., near the patient interface 3000) of the air circuit 4170. For example, the antenna 9100 may be disposed within a portion of the air circuit 4170, such as, for example, within a cover 4172 on a proximal portion 4170A of the air circuit 4170, as shown in FIGS. 6A and 6B and to be described in further detail below. In alternative embodiments, the antenna 9100 may be disposed within a lumen 4178 (see FIGS. 6A and 6B) of air circuit 4170. In further alternative configurations, the antenna 9100 may be fixedly or removably coupled to an external portion of the air circuit 4170, such as, for example, on an external surface of the air circuit 4170.

The tag 9200 is configured to emit radio waves to transmit information. The tag 9200 may contain a microchip that stores and processes information such as, for example, the unique identifier of the tag 9200 and the antenna 9100 to enable the tag 9200 to receive and/or transmit radio signals. The tag 9200 may be an active tag or a passive tag. If a passive tag is used, it may rely on the power of the antenna to transmit data, and thus may have a shorter transmittal range. For example, the tag 9200 may receive all of the required energy from a magnetic field in which the tag 9200 operates.

The tag 9200 may be read-only, read/write, or write once, read many. The tag 9200 may be configured to include identifying data of the patient interface 3000 and/or the patient. In some aspects, the tag 9200 may include information related to a date of usage or a time stamp. The tag 9200 may additionally or alternatively include information related to the type of patient interface 3000 being used, characteristics of patient interface 3000 (e.g., one or more of cushion material, cushion size, conduit size, patient interface size, length of use of the patient interface, date of manufacture of the patient interface, a set of respiratory therapy conditions the patient interface is suitable for use with, etc.), a serial identification number of the patient interface 3000, a production batch identification number of the patient interface 3000, and/or other aspects of the respiratory therapy system 8000. In some aspects, the tag 9200 may be configured to include patient information, e.g., the type of therapy or therapy settings the patient is intended to receive, or other information. The tag 9200 may also be used to detect a connection and/or a disconnection of the air circuit 4170 and/or an accessory device (not shown).

In some embodiments, the tag 9200 may include an adhesive, e.g., to assist in applying the tag 9200 to the patient interface 3000 and/or maintaining the tag 9200 in place on the patient interface 3000 after application. The tag 9200 may alternatively be overmolded within a portion of patient interface 3000. For example, the tag 9200 may be overmolded into a soft plastic material, e.g., a silicone cushion, or into a hard plastic material, e.g., a plastic frame of the patient interface 3000. Alternatively, the tag 9200 may comprise conductive silicone and/or conductive thread(s) or ink(s) (e.g., silver ink) printed on the soft and/or hard plastic material comprising patient interface 3000. The tag 9200 may include, e.g., overmolded inlay label, conductive textile, or may be coupled to the patient interface 3000 in any suitable method used in the art.

The tag 9200 may be an RFID tag, and, in some instances, may be a near-field communication (NFC) tag. The tag 9200 may be configured to generate an electromagnetic field with a frequency of about 10 MHz to about 12 GHz (e.g., about 13.56 megahertz (MHz)), and the antenna 9100 may be configured to read data transmitted from tag 9200 at about 10 MHz to about 12 GHz (e.g., about 13.56 MHz). In other aspects, the RFID system 9000 may alternatively be configured to generate an electromagnetic field with a frequency between about 30 kilohertz (kHz) and about 3 gigahertz (GHz). In some examples, RFID system 9000 may be configured to operate in a low frequency range, or between about 30 kHz to 300 kHz. Accordingly, the RFID system 9000 may be configured to have a read range of up to approximately 10 centimetres (approximately 3.94 inches), although the exact distance may vary depending, e.g., on the angles of the various components in the system relative to one another. Alternatively, the RFID system 9000 may be configured to operate in a high frequency range, or between about 3 MHz and 30 MHz. In such a configuration, the RFID system 9000 may be configured to have a read range between approximately 10 centimetres up to approximately one metre (approximately 3.94 inches to approximately 39.37 inches). Further still, the RFID system 9000 may be configured to operate in an ultra-high frequency range, or between about 300 MHz and 3 GHz. In such a configuration, the RFID system 9000 may be configured to have a read range between approximately one metre up to approximately 12 metres. For example, the tag 9200 may be an ultra-high frequency (UHF) tag, a Bluetooth tag, or an ultra-wideband (UWB) tag. The tag 9200 and the antenna 9100 may be tuned to operate on the same frequency. A UWB tag 9200 may operate at a frequency range of about 3.1 GHz to about 10.6 GHz, a Bluetooth tag 9200 may operate at a frequency of about 2.4 GHz, and a UHF tag 9200 may operate at a frequency of about 300 MHz to about 3 GHz.

In some examples, a shorter read range for the RFID system 9000 may be desirable. For example, if the patient is in the vicinity of multiple device or objects containing RFID components, the RFID system 9000 may inadvertently read the tag or antenna on the peripheral devices. Accordingly, if the RFID system 9000 is configured to operate on a low frequency range, e.g., at 13.56 MHz, the possibility for the RFID system 9000 to read a peripheral device inadvertently is decreased.

The RFID system 9000 may include a single tag 9200 fixedly or removably coupled to patient interface 3000 or to an accessory device (not shown). Alternatively, the RFID system 9000 may include two or more of the tags 9200 fixedly coupled to the patient interface 3000 or to an accessory device (not shown). The RFID tags 9200 described herein may be off-the-shelf components or may be customized according to a size and/or a shape of patient interface 3000 and/or according to a desired read range for tag 9200. Although tag 9200 is described herein as being associated with the patient interface 3000, tag 9200 may alternatively or additionally be associated with, for example, the air circuit 4170 or another accessory of respiratory therapy system 8000. To the extent that tag 9200 is associated with air circuit 4170 or another accessory, tag 9200 may include information about the accessory or air circuit with which it is associated.

The antenna 9100 is configured to receive data from the tag 9200 and transmit the received data to the transceiver, or reader, 9300. The antenna 9100 may be set to the same inductance for the tags 9200 for different types of the patient interface 3000 so that the antenna 9100 may be compatible with a variety of patient interfaces 3000. The transceiver 9300 may be operably connected to the antenna 9100 physically (e.g., via a wire) and may be located on or in the air circuit 4170 or the adapter 9400. In one configuration, the transceiver 9300 may be located on or in the RPT device 4000. In one configuration, the transceiver 9300 may be external to the respiratory therapy system 8000. For example, the transceiver 9300 may be a scanner, a smartphone, a tablet, or any other device configured to receive transmitted RFID signals from an RFID tag or antenna. Accordingly, the antenna 9100 is configured to communicate data received from the tag 9200 to the transceiver 9300. The transceiver 9300 may also be configured to save or store the transmitted data from the antenna 9100.

Referring still to FIG. 5A, the transceiver 9300 may include a controller on a flexible circuit, for example, attached within the air circuit 4170. The transceiver 9300 may relay information from the antenna 9100 to a controller configured to control the RPT device 4000. The controller may be separate from the RPT device 4000 or may be incorporated as part of the RPT device 4000. The controller may function as described above. The transceiver 9300 may generate an electromagnetic field with an appropriate frequency, e.g., of 13.56 MHz, and may be configured to read the tag 9200 from patient interface 3000 or an accessory coupled at the patient interface 3000. Accessory parts may include, but are not limited to, e.g., one or more of headgear of the patient interface, cushion, heat and moisture exchanger or waterless humidifiers, an air filter, an air conduit, or an adapter, as described above.

In some configurations, the transceiver 9300 may be configured to transmit data to the RPT device 4000 physically (e.g., via a wire) or wirelessly. The RPT device 4000 may also be configured to save or store the transmitted data from transceiver 9300, interpret the transmitted data, and/or transmit an alert or signal to the user or care provider, as described above. In some embodiments, the RPT device 4000 may also be configured to automatically change one or more characteristics of the respiratory pressure therapy, for example, based on the raw data and/or interpreted data received from the transceiver 9300. Additionally or alternatively, the RPT device 4000 may be configured to suggest one or more therapy settings, for example, to promote patient care and/or patient comfort based on the interpreted data from the transceiver 9300, as will be discussed further below.

FIG. 5B illustrates an alternative configuration of a respiratory therapy system 8000′. The respiratory therapy system 8000′ may include any characteristics discussed above with respect to the respiratory therapy system 8000, except as described below. For example, the respiratory therapy system 8000′ includes patient interface 3000 and respiratory pressure therapy (RPT) device 4000 fluidly coupled by the air circuit 4170. The respiratory therapy system 8000′ further comprises an RFID system 9000′.

Similarly to the RFID system 9000 discussed above with respect to FIG. 5A, the RFID system 9000′ comprises a tag 9200, an antenna 9100, and a transceiver 9300. Comparatively, in the configuration shown in FIG. 5B, an adapter 9400 is configured to be fixedly or removably coupled between air circuit 4170 and the patient interface 3000. The adapter 9400 may comprise or contain antenna the 9100. Accordingly, the adapter 9400, and thus the antenna 9100, may be a separate component, for example, that is independent of air circuit 4170 and patient interface 3000. In such a way, the adapter 9400 may be used in conjunction with a variety of air circuits 4170 or may be used to retrofit an air circuit 4170 without the antenna 9100. In some aspects, the adapter 9400 may be reusable, and the air circuit 4170 may be replaceable. The antenna shown in FIG. 5B may have any or all of the characteristics of the antenna 9100, described above with respect to FIG. 5A. In some aspects of the technology, the adapter 9400 may also include one or more sensors.

Various types of sensors may be used in connection with the techniques described herein. Any or all of the sensors may be configured to generate a signal based on, indicative of, or reflective of one or more physical phenomenon. Illustrative examples of sensor types (and the physical phenomenon that is sensed) include humidity, temperature, air flow, pressure, light, particle, biochemical, acceleration, angular velocity, and the like. Other types of sensors are also discussed herein and may be used in connection with the techniques described herein. Any or all of the sensors described herein may communicate signals that are based on the sensed physical phenomenon either via wired communication and/or via wireless communication. Any or all of the sensors discussed herein may be included as part of adapter 9400 (e.g., as sensor 9504), which is discussed in greater detail elsewhere herein. Any or all of the sensors described herein may be located at different locations in connection with an example respiratory therapy system and components thereof. For example, a first sensor may be located in the RPT device, another in the patient interface, and another in an air conduit. Other examples of where one or more sensors may be located are described herein.

The adapter 9400 may be in fluid communication with the air circuit 4170 and the patient interface 3000. In such a way, the adapter 9400 may include a lumen having a proximal opening and a distal opening. The lumen is configured to permit air flow from the air circuit 4170, through the adapter 9400, into the patient interface 3000. Additionally, the adapter 9400 forms a fluid-tight seal between the air circuit 4170 and the patient interface 3000. To achieve the fluid tight seal, in some configurations, a proximal portion of the air circuit 4170 may extend at least partially into a distal portion of the adapter 9400, for example, through the distal opening of the adapter 9400. Alternatively, a distal portion of the adapter 9400 may extend at least partially into the proximal portion of the air circuit 4170. Similarly, a distal portion of the patient interface 3000 may extend at least partially into the proximal opening of the adapter 9400.

FIGS. 6A and 6B illustrate perspective views of a proximal portion 4170A of the air circuit 4170. For example, the air circuit 4170 shown in FIGS. 6A and 6B may be used with the system 8000 or the system 8000′ shown in FIGS. 5A and 5B, respectively. The air circuit 4170 is a conduit or a tube constructed and arranged to allow, in use, a flow of air to travel between two components of a respiratory therapy system such as, for example, the RPT device 4000 and the patient interface 3000. Although not shown, in some cases, there may be separate limbs of the air circuit 4170 for inhalation and exhalation. In other cases, the air circuit 4170 may be single-limbed and used for both inhalation and exhalation.

The proximal portion 4170A of the air circuit 4170 may include a proximal-most end comprising a cover 4172. The cover 4172 may be directly or indirectly coupled to the proximal portion 4170A of air circuit 4170. The cover 4172 may include one or more features 4174 extending outwardly from a proximal face 4172A of the cover 4172. The feature(s) 4174 may be configured to facilitate a secure and/or fluid-tight connection, for example, between the air circuit 4170 and the patient interface 3000 (shown in FIG. 5A) or between the air circuit 4170 and the adapter 9400 (as shown in FIG. 5B). For example, the feature 4174 may comprise a bayonet-style connector including protrusions and/or grooves configured to engage with corresponding protrusions and/or grooves on the patient interface/adapter by turning the connector until it latches. In an alternative example, the feature 4174 may comprise an iso-taper configured to connect to the patient interface/adapter. However, it should be appreciated that the feature 4174 may include other suitable configurations (e.g., releasable snap-fit connector) to facilitate connection to the patient interface/adapter.

The cover 4172 further includes an opening 4176. The opening 4176 extends through an entirety of the cover 4172 and is in fluid communication with the lumen 4178 of the air circuit 4170. The cover 4172 and/or an internal surface defining the lumen 4178 of the air circuit 4170 may include one or more projections, or protrusions, 4190. The protrusion 4190 may extend radially inward, for example, from the internal surface defining the lumen 4178 and/or from an internal surface 4179 of the cover 4172 into the lumen 4178. The protrusion 4190 may extend partially or completely through the lumen 4178. As discussed below, with respect to FIG. 6B, the protrusion 4190 may include or may mate with an encapsulation 4199. Although not shown, the protrusion 4190 may comprise one or more sensors. The sensor(s) may be used to identify, characterize, or define various aspects of the fluid (e.g., air, gas, etc.) within the air circuit 4170. For example, the protrusion 4190 may comprise sensor(s) to identify a temperature, humidity level, air flow, pressure, etc., of the fluid within the air circuit 4170. The sensor(s) may be in communication with the RPT device 4000, antenna 9100, and/or transceiver 9300.

FIG. 6B illustrates proximal portion 4170A of a version of the air circuit 4170 with the cover 4172 removed. A circuit board 4192 may be disposed within the cover 4172, for example, between an outward-facing surface of the air circuit 4170 and an inward-facing surface of the cover 4172. The circuit board 4192 may be configured to surround a projection 4170C extending proximally from a proximal-most surface 4170B of the air circuit 4170. Although not shown, the projection 4170C may include one or more features (e.g., indentation(s), protrusion(s), etc.) to assist with supporting and/or restraining the circuit board 4192 in position (e.g., around the projection 4170C). For example, the projection 4170C may include a first feature (e.g., indentation, protrusion) to couple a proximal portion 4192A of the circuit board 4192 to the projection 4170C and/or a second feature (e.g., indentation, protrusion) to couple a distal portion 4192B of the circuit board 4192 to the projection 4170C.

A surface 4195 of the circuit board 4192 defining a platform 4196 is configured to face proximally. For example, the surface 4195 is configured to lie in a plane that is approximately perpendicular to an axis A extending longitudinally from a proximal end to a distal end of the air circuit 4170. In such a way, the platform 4196 is bent approximately 90 degrees relative to the circuit board 4192. Platform 4196 is coupled to the circuit board 4192 via a bridge 4194. The platform 4196 further comprises the antenna 4100. The antenna 4100 may have any or all of the characteristics of the antenna 9100 described above with respect to FIGS. 5A and 5B. The platform 4196 comprises an opening 4198 extending through the entirety of the platform 4196. The opening 4198 may be configured to permit fluid flow through the lumen 4178 of the air circuit 4170. Accordingly, the opening 4198 may have the same diameter or a different diameter than the lumen 4178 of air circuit 4170. While the cover 4172 is removed from FIG. 6B to show this example arrangement of the antenna 4100 incorporated into the air circuit 4170, during use, the cover 4172 would obscure the antenna 4100 from view, as shown in FIG. 6A.

The encapsulation 4199 may house one or more sensors, such as those discussed above (e.g., the sensors in the protrusion 4190). In some examples, the encapsulation 4199 may be formed from additive manufacturing techniques (e.g., 3D printing). The encapsulation 4199 may be formed of any suitable material, such as, for example, a transparent material, such as silicone. The encapsulation 4199 may enclose and seal the sensor and keep the sensor in place. In alternatives, a single encapsulation 4199 may enclose multiple sensors. In further alternatives, the proximal portion 4170A may include multiple encapsulations 4199, each enclosing one or more sensors.

FIG. 6C illustrates a front view of the circuit board 4192 in a first, or flattened, configuration. As previously described with regards to FIG. 6B, the circuit board 4192 includes the proximal portion 4192A and the distal portion 4192B. The circuit board 4192 is configured to bend or flex around the projection 4170C of the air circuit 4170 (shown in FIG. 6B). Accordingly, circuit board 4192 may be comprised of one or more bendable or flexible materials.

The bridge 4194 is configured to provide a physical connection between the circuit board 4192 and the platform 4196. To enable the platform 4196 to bend approximately 90 degrees, as shown in FIG. 6B, the bridge 4194 is formed of a bendable or flexible material. The bridge 4194 may have the same thickness as the circuit board 4192 and/or the platform 4196, or may have a lesser thickness than the circuit board 4192 and/or the platform 4196. A lesser thickness of the bridge 4194 may permit the bridge 4194 to bend to a greater extent and/or to bend more easily. Additionally or alternatively, the bridge 4194 may be comprised of the same material or of a different material than the circuit board 4192 and/or the platform 4196. For example, the bridge 4194 may be comprised of a material having more flexible characteristics as compared to the circuit board 4192 and/or the platform 4196. When applied to the air circuit 4170, the opening 4198 of the platform 4196 may be positioned coaxially with the lumen 4178 of the air circuit 4170 so that the platform 4196 sits on a proximal end of the projection 4170C. The bridge 4194 may be bent relative to the platform 4196, and the circuit board 4192 may be bent to wrap around the projection 4170C, as shown in FIG. 6B.

The antenna 4100 is fixed to the platform 4196 and is electrically coupled to the circuit board 4192, for example, via the bridge 4194. The antenna 4100 surrounds or encircles the opening 4198. Accordingly, the antenna 4100 surrounds or encircles the proximal opening of the lumen 4178 of the air circuit 4170, as shown in FIG. 6B.

The antenna 4100 may be formed via a variety of methods or combinations of methods. The antenna 4100 may include flexible circuits that connect directly to the main flexible circuit of the transceiver 9300 which may connect to the air circuit 4170. The antenna 4100 may be formed from one or more conductive, e.g., copper, wires welded, soldered, glued, or otherwise affixed to the platform 4196 or around a periphery of the air circuit 4170. The conductive wires may be affixed to the transceiver module of the circuit board. The antenna 4100 may alternatively be formed via printed conductive, e.g., silver ink or printed conductive thread, for example, applied directly onto the platform 4196 or around a periphery of the air circuit 4170. The antenna 4100 may be formed of flexible printed circuit (FPC), a coil wire, or conductive printing or plastic. Laser direct structuring (LDS) may be applied onto a plastic encasing that may protect the electronic components. LDS may be coupled, welded, or soldered, e.g., to the transceiver module of the circuit board. In such a way, for example, the antenna 4100 may be implemented directly onto platform 4196.

The strength of a magnetic field of a passive tag 9200 is directly proportionate to the distance between the passive tag 9200 and the antenna 9100. For example, the strength of the magnetic field of the passive RFID tag 9200 decreases as the tag 9200 is moved away from the antenna 9100. Conversely, the strength of the magnetic field of the passive tag 9200 increases as the tag 9200 is moved closer to the antenna 9100. This is because, as discussed above, the passive tags 9200 acquire the requisite energy to transmit the radio waves from a magnetic field in which they operate. In other words, the passive tag 9200 and the antenna 9100 operate according to the principle of inductive coupling. RFID system 9000 shown in FIGS. 5A and 5B operates under these conditions. Accordingly, the placement of the antenna 9100 relative to the tag 9200 impacts the operability of the RFID system 9000 within the system 8000.

As previously discussed, the tag 9200 may be fixedly or removably coupled to the patient interface 3000, for example, near where the air circuit 4170 is coupled to the patient interface 3000. The distance between the antenna 9100 and the tag 9200 may vary depending on the type or size of the patient interface 3000 used by a patient. As shown in FIGS. 4J-4P and described above, the patient interface 3000 may have various different configurations and various components. The type or location of the tag 9200 may be customized for different patient interface designs, for example, to fit in the antenna 9100 or place the tag 9200 within an appropriate reading orientation or angle. The antenna 9100 and the tag 9200 may be positioned close to where the air circuit 4170 couples to the patient interface 3000 and may be positioned to avoid contact with a patient's skin during use, or to avoid a vent contact area. The systems 8000 described herein may be used on a variety of patient interface types, such as, for example, a tube-up nasal patient interface (e.g., having any of the properties of the patient interface 3000-3 of FIG. 4L), a tube-down nasal patient interface (e.g., having any properties of the patient interface 3000-4 of FIG. 4M), a tube-up full-face patient interface (e.g., having any of the properties of the patient interface 3000-1 of FIG. 4J), or a tube-down full-face patient interface (e.g., having any of the properties of the patient interface 3000-2 of FIG. 4K).

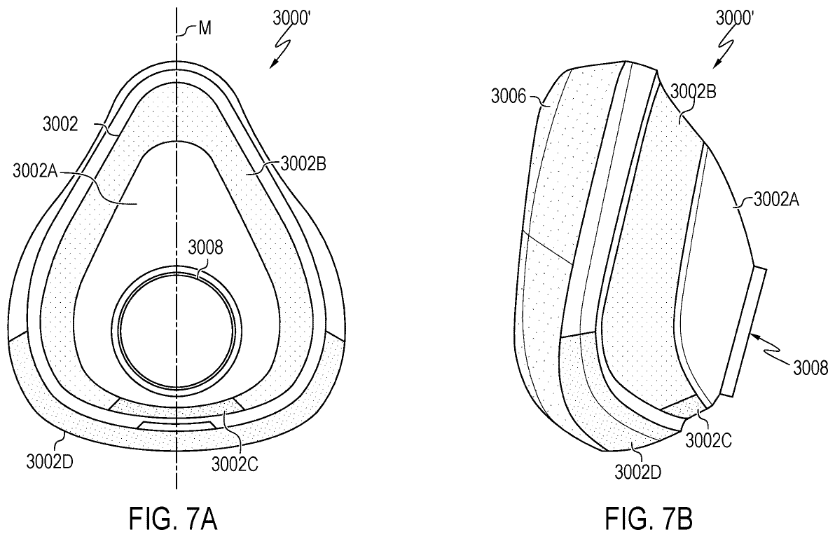

FIGS. 7A and 7B depict an exemplary patient interface 3000′. The patient interface 3000′ may be used, for example, as the patient interface 3000 of the system 8000 or the system 8000′ described above with respect to FIGS. 5A and 5B, respectively. FIG. 7A is a front view of the patient interface 3000′, and FIG. 7B is a side view of the patient interface 3000′. The patient interface 3000′ may be commonly known as a nasal patient interface or a full-face patient interface. For example, the patient interface 3000′ may be configured to cover a patient's mouth and nose, or just a patient's nose.

FIGS. 7A and 7B include stippling to better illustrate and describe the various portions of the patient interface 3000′ to be described herein. However, the stippling is for purposes of description and does not necessarily represent that the different portions of the patient interface 3000′ include different materials, properties, or color, although in one example, the different portions may include different materials, properties, or colors.

The patient interface 3000′ has a three-dimensional shape that may vary depending on the style or type of patient interface. For example, the size and shape of the patient interface 3000′ may vary considerably from the size and shape depicted in FIGS. 7A and 7B. Portions of the patient interface 3000′ may be formed of a rigid or hard material, and other portions of the patient interface 3000′ may be formed of a soft or pliable material. Accordingly, the placement of an RFID tag, such as the tag 9200, on the patient interface 3000′ poses unique considerations.

The patient interface 3000′ comprises a shell 3002 having a first portion 3002A, a second portion 3002B, a third portion 3002C, and a fourth portion 3002D. One or more portions of the shell 3002 (e.g., the first portion 3002A, the second portion 3002B, and the third portion 3002C) may be formed of a rigid material (e.g., polycarbonate) and may not contact the patient's skin. One or more portions of the shell 3002 (e.g., fourth portion 3002D) may be formed of a softer, more pliable material (e.g., silicone rubber). First portion 3002A includes an opening 3008. The opening 3008 extends an entire thickness of the shell 3002 and is configured to directly or indirectly couple the patient interface 3000′ to a proximal portion of the air circuit 4170. The opening 3008 is aligned with a median plane M of patient interface. The first portion 3002A surrounds the opening 3008. The second portion 3002B and the third portion 3002C surround the first portion 3002A.

Because the antenna 9100 (4100 in FIGS. 6B, 6C) may be disposed in or on a proximal portion of the air circuit 4170 and the proximal portion of the air circuit 4170 is coupled to the patient interface 3000′ via the opening 3008, the tag 9200 may be positioned near the opening 3008 in a “tube-down” configuration of the patient interface 3000′, as shown in FIG. 7A (FIGS. 4K and 4M also depict similar “tube down” configurations for which the tag 9200 may have a similar position). For example, the tag 9200 may be coupled to either the first portion 3002A, the second portion 3002B, the third portion 3002C, or the fourth portion 3002D. In some aspects, due to the proximity of the opening 3008, the tag 9200 may be placed on or near the third portion 3002C, which may be an ideal positioning for the patient interface 3000′. The third portion 3002C is near or adjacent to median plane M. For example, the tag 9200 may be placed to one side (i.e., a left or right side) defined by median plane M, or to the other side (i.e., a right or left side) defined by median plan M, or along median plan M. In some configurations, the tag 9200 may be positioned so as to straddle multiple portions of the patient interface 3000′. For example, a first portion of tag 9200 may be on first portion 3002A of the shell 3002 and a second portion of tag 9200 is on the second portion 3002B and/or the third portion 3002C of the shell 3002. In such a way, the placement of the tag 9200 may vary between the first portion 3002A, the second portion 3002B, the third portion 3002C and/or the fourth portion 3002D.

The tag 9200 may be placed on or in the shell 3002 through a variety of means. For example, the tag 9200 may be a chip overmolded into the shell 3002. Alternatively, the tag 9200 may be printed directly on the shell 3002 via printed conductive material, e.g., silver ink. The tag 9200 may be an inlay label overmolded into shell 3002. In such an example, the tag 9200 (e.g., the inlay label) may be inserted, e.g., via automation into a cavity of a mold for the shell 3002, and an injection mold and plastic may be injected over the inlay label, thus fixing inlay label within or on the shell 3002. Alternatively, the tag 9200 may be formed of conductive silicone and/or conductive threads printed on the shell 3002. In further alternatives, the tag 9200 may be formed of a conductive textile with stretchable copper traces. Accordingly, the tag 9200 may be formed by any one of these methods or by any combination of these methods, including those commonly known in the art. Further still, the tag 9200 may be an individual component fixed to the shell 3002 through a variety of suitable means commonly known in the art, including, but not limited to adhesives, and/or one or more mechanical fasteners.

The patient interface 3000′ further comprises a cushion 3006 configured to contact the patient's face. The cushion 3006 may be formed of a soft or pliable material, for example, silicone rubber. The cushion 3006 is configured to form a seal against the patient's face. Although the tag 9200 may be placed on or within the cushion 3006, placing the tag 9200 on or within the cushion 3006 may be less desirable due to challenges associated with the distance of cushion 3006 from the opening 3008 and/or challenges associated with one or more characteristics of the material comprising the cushion 3006 (e.g., the material's flexibility, softness, thickness, etc.). Additionally, or alternatively, placing the tag 9200 on or within the cushion 3006, for example, between the patient's skin and the cushion 3006 may result in patient discomfort. That said, it may be possible to locate the tag 9200 in the cushion 3006.

Although FIGS. 7A and 7B depict a tube-down patient interface 3000′ and discuss location of the tag 9200 near the opening 3008, a tube-up patient interface may also be used according to this technology. In a tube-up patient interface 3000′, the location of the tag 9200 may be, for example, located closer to an upper region of the patient interface 3000′. In some aspects, the location of the tag 9200 on the patient interface 3000′ may, at least in part, depend on whether a tube-up or tube-down configuration is utilized with the patient interface 3000′ so as to position the tag 9200 to be closer to the proximal portion of the air circuit 4170 and the antenna 9100 when attached to the patient interface 3000′.

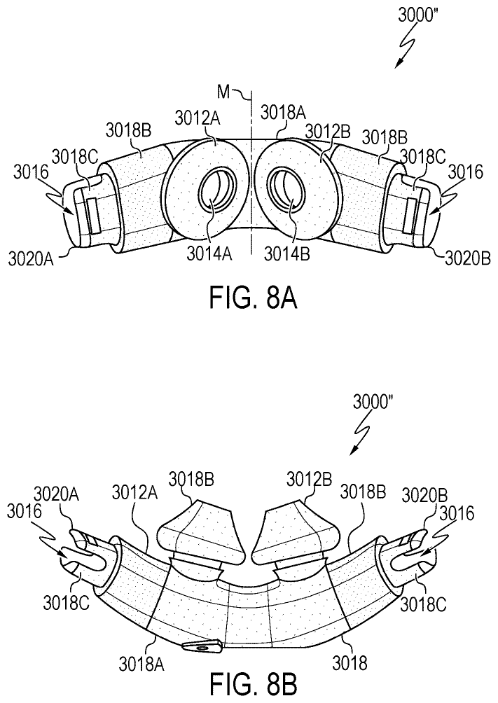

FIGS. 8A and 8B depict an alternative exemplary patient interface 3000″. The patient interface 3000″ may be used, for example, with the system 8000 or the system 8000′ described above with respect to FIGS. 5A and 5B, respectively. FIG. 8A illustrates a back view of the patient interface 3000″, and FIG. 8B illustrates a top view of the patient interface 3000″. The patient interface 3000′ may be commonly known as a nasal pillow. For example, the patient interface 3000″ may be configured to be partially inserted into a patient's nose.

FIGS. 8A and 8B include stippling to better illustrate and describe the various portions of the patient interface 3000″ to be described herein. However, the stippling is for purposes of description and does not necessarily represent that the different portions of the patient interface 3000″ include different materials, properties, or color, although in one example, the different portions may include different materials, properties, or colors.

The patient interface 3000″ has a complex three-dimensional shape. For example, the patient interface 3000″ includes a variety of curves and contours. Portions of the patient interface 3000″ may be formed of a soft or pliable material, and other portions of the patient interface 3000″ may be formed of hard or rigid material. For example, portions of the patient interface 3000″ contacting the patient's face may be soft or pliable, and portions used to facilitate a connection between the air circuit 4170 and the patient interface 3000″ may be hard, or rigid. Additionally, the patient interface 3000″ is smaller in size as compared to the patient interface 3000′, described above. Accordingly, the placement of an RFID tag, such as the tag 9200, on the patient interface 3000″ poses unique considerations.

The patient interface 3000″ includes a first nasal cushion 3012A and a second nasal cushion 3012B on either side of a median plane M. For example, the first nasal cushion 3012A is on a first (e.g., left or right side) or median plane M, and second nasal cushion is on a second (e.g., right or left side) of median plane M. The first nasal cushion 3012A and second nasal cushion 3012B are configured to be at least partially inserted into the patient's nostrils and are thus skin-contacting. Accordingly, the material comprising first nasal cushion 3012A and second nasal cushion 3012B may be soft and pliable (e.g., silicone rubber). Additionally, the first nasal cushion 3012A and the second nasal cushion 3012B include a first hole 3014A and a second hole 3014B, respectively, extending through an entire thickness of the first nasal cushion 3012A and the second nasal cushion 3012B such that the first nasal cushion 3012A and the second nasal cushion 3012B are in fluid connection with a lumen 3016 of tubular portion 3018. The lumen 3016 extends through the tubular portion 3018, for example, from a first side 3020A of the tubular portion 3018 to a second side 3020B of the tubular portion 3018. The tubular portion 3018 includes a first portion 3018A, a second portion 3018B, and a third portion 3018C.

The first nasal cushion 3012A and the second nasal cushion 3012B extend radially outward from the first portion 3018A. The first portion 3018A may be comprised of the same material as the first nasal cushion 3012A and second nasal cushion 3012B or of a different material. The first nasal cushion 3012A and the second nasal cushion 3012B are configured to form a seal between the patient's skin.

The second portion 3018B is on either side of the first portion 3018A. For example, the second portion 3018B is to the left and to the right of the first portion 3018A. The second portion 3018B may be comprised of the same material as the first portion 3018A or comprised of a different material. The second portion 3018B is not configured to form a seal with the patient's skin and may not be configured to contact the patient's skin.

The third portion 3018C is located on the outer sides of each second portion 3018B. For example, the third portion 3018C is to the left of the first portion 3018A and to the right of the second portion 3018B. The third portion 3018C comprises the first side 3020A and the second side 3020B. The third portion 3018C may not be configured to contact the patient's skin. Accordingly, the third portion 3018C may be comprised of a harder or more rigid material. In a tube-down patient interface 3000″, the air circuit 4170 may connect to patient interface 3000″ at the first portion 3018A, generally opposite the first nasal cushion 3012A and the second nasal cushion 3012B. Alternatively, in a tube-up patient interface 3000″, the air circuit 4170 may attach to conduit headgear, which may attach to the third portions 3018C. In some aspects, the location of tag 9200 on patient interface 3000″ may, at least in part, depend on whether a tube-up or tube-down configuration is utilized with patient interface 3000″ so as to position tag 9200 to be closer to the proximal portion of air circuit 4170 and antenna 9100 when attached to patient interface 3000″.

In utilizing the patient interface 3000″, the air circuit 4170 with the antenna 9100 may be connected to the patient interface 3000″. The tag 9200 may be positioned near the connection site of the air circuit 4170. For example, the tag 9200 may be coupled to the first portion 3018A, the second portion 3018B, or the third portion 3018C. In some aspects, in order to avoid contact with a patient's skin, the tag 9200 may be coupled to the second portion 3018B or the third portion 3018C. In some configurations, the tag 9200 may be positioned so as to straddle multiple portions of the patient interface 3000′. For example, a first portion of the tag 9200 may be on the second portion 3018B, and a second portion of the tag 9200 may be on the third portion 3018C. In such a way, the placement of the tag 9200 may vary between the first portion 3018A, the second portion 3018B, and the third portion 3018C.

Placing the tag 9200 on or within the first nasal cushion 3012A, the second nasal cushion 3012B, and/or, in some aspects, the first portion 3018A may be less desirable due to challenges associated with contact with a patient's skin. Additional or alternative challenges may be associated with one or more characteristics of the material comprising first nasal cushion 3012A and the second nasal cushion 3012B (e.g., the material's flexibility, softness, thickness, etc.). Additionally or alternatively, placing the tag 9200 on or within the first nasal cushion 3012A and the second nasal cushion 3012B, for example, between the patient's skin and first nasal cushion 3012A or the second nasal cushion 3012B, may result in patient discomfort.

The tag 9200 may be placed on or in the patient interface 3000″ through a variety of means, similar to those discussed above with fixing the tag 9200 to the patient interface 3000′. For example, the tag 9200 may be a chip overmolded within the tubular portion 3018. Alternatively, the tag 9200 may be printed directly onto the tubular portion 3018 via printed conductive, e.g., silver, ink. The tag 9200 may be an inlay label overmolded within the tubular portion 3018. In such an example, the tag 9200 (e.g., the inlay label) may be inserted, e.g., via automation into a cavity of a mold for the tubular portion 3018 and an injection mold and plastic may be injected over the inlay label, thus fixing the inlay label within or on the tubular portion 3018. Alternatively, the tag 9200 may be formed of conductive silicone and/or conductive threads printed on the tubular portion 3018. In further alternatives, the tag 9200 may be formed of a conductive textile with stretchable conductive, e.g., copper, traces. Accordingly, the tag 9200 may be formed by any one of these methods or by any combination of these methods, including those commonly known in the art. Further still, the tag 9200 may be an individual component fixed to the tubular portion 3018 through a variety of suitable means commonly known in the art, including, but not limited to adhesives, and/or one or more mechanical fasteners.

During use of the patient interfaces 3000, 3000′, 3000″, or another suitable patient interface (collectively patient interface, in which aspects described in connection with patient interface 3000 may be similarly applied to patient interfaces 3000′ and/or 3000″), the air circuit 4170 may be fluidly coupled to the RPT device 4000 and to the patient interface 3000. As described above, the RPT device 4000 may be configured to supply a flow of gas, for example, air, which may be supplemented with oxygen, through the air circuit 4170 to the patient interface 3000. The RPT device 4000 may also be configured to receive a signal from RFID system 9000 once air circuit 4170 is coupled to patient interface 3000 (e.g., via the data communication interface 4280 and/or the central controller 4230). The signal may include information regarding patient interface 3000 or an accessory. For example, the antenna 9100 in the air circuit 4170 or the adapter 9400 may the detect tag 9200 in the patient interface 3000 and may read information about the patient interface 3000 associated with the tag 9200. Via the transceiver 9300, for example, this information may be transmitted to the RPT device 4000 (e.g., to the data communication interface 4280).

In some aspects, the received information may be one or more of e.g., the type of the patient interface 3000 being used, characteristics of the patient interface 3000 (e.g., one or more of cushion material, cushion size, conduit size, patient interface size, length of use of the patient interface, date of manufacture of the patient interface, a set of respiratory therapy conditions the patient interface is suitable for use with, etc.), date of usage or time stamp, batch identification number of the patient interface 3000, or a serial identification number of the patient interface 3000. In some aspects, the received information may be one or more of patient information, e.g., the type of therapy or therapy settings the patient is intended to receive, or other information. In some aspects, the received information may be whether the air circuit 4170 and/or an accessory device (not shown) is connected or disconnected from the patient interface 3000.

The RPT device 4000 may be configured to perform an action upon receiving information from the RFID system 9000 about the patient interface 3000. To facilitate the description herein, RPT the device 4000 may be described as performing an action based on receipt of information, however, this may mean either that a controller incorporated as part of the RPT device 4000 (e.g., the central controller 4230 and/or the therapy control module 4330) may cause RPT device to perform an action or a controller separate from the RPT device 4000 may cause the RPT device 4000 to perform an action. For example, the RPT device 4000 may automatically control operation of the therapy provided to the patient based on the information or signal received (e.g., via the central controller 4230 and/or the therapy control module 4330, as discussed above). For example, the breathing experience may be improved by determining whether the settings of the RPT device (e.g., gas flow, humidity levels, etc.) are correctly aligned to the patient interface being worn by the patient. In some configurations, the RPT device 4000 may enable the design of patient interface-specific venting and flow curves to promote more comfortable and/or efficient delivery of therapy. In some aspects of the technology, an indication to the patient may be generated based on the information received by the RPT device 4000. For example, an indication may be generated that the incorrect type or size of patient interface is being used, that a cushion of the patient interface or the entire patient interface should be replaced, that the RPT device 4000 is configured with one or more incorrect settings that should be changed by the patient, or other suitable indications. The indication may be generated, e.g., on one or more of a display of the RPT device 4000 or other component of the system 8000, to an external device, such as the remote external device 4286 or the local external device 4288 (e.g., a patient's tablet, smartphone, or computer, or a device of a healthcare provider). In other aspects, the respiratory therapy system 8000 may receive input from a patient, for example, a patient may input information regarding the patient interface 3000 during a therapy session, and the received input from the patient may be compared with the information received from the patient interface 3000 in the RFID system 9000 to confirm the accuracy of a patient's input. In some aspects, information received from the RFID system 9000 may be used to track usage or other characteristics of the patient interface in use.

In further aspects of the technology, information received from the patient interface 3000 may be received by the RPT device 4000 in addition to information received from other sensors or systems associated with the system 8000 or the system 8000′ (collectively the system, in which aspects described in connection with system 8000 may be similarly applied to system 8000′). For example, sensors or systems configured to detect air flow, pressure, air leaks, humidity, or other characteristics of the system 8000 may transmit information to the RPT device 4000. The RPT device 4000 may analyse or interpret information regarding the patient interface 3000 received from the RFID system 9000, 9000′ (collectively RFID system, in which aspects described in connection with RFID system 9000 may be similarly applied to RFID system 9000′) in combination with one or more other sensors. For example, information may be received from the RFID system 9000 regarding the type or size of patient interface being worn or how long the patient interface has been used for. The system 8000 may also receive information from other sensors or patient input regarding patient interface discomfort or the occurrence of leaks.

The system 8000 may analyse this information together and may take an action based on the aggregate information. For example, if patient interface discomfort is indicated by a patient or leaks are detected with the patient interface, based on the information received from the RFID system 9000, the RPT device 4000 may generate an indication to a patient that a difference size or type of patient interface should be used, a different size or type of cushion should be used, or a new patient interface or cushion should be used. This indication may include, e.g., a suggestion regarding a patient interface or cushion type or size to be used or guidance on how to select a better-fitting cushion or patient interface type or size. Indications may be generated as described above, e.g., on one or more of a display of the RPT device 4000 (e.g., the display 4294) or other component of the system 8000, to an external device, such as the remote external device 4286 or the local external device 4288 (e.g., a patient's tablet, smartphone, or computer, or a device of a healthcare provider). In other aspects, one or more settings of the RPT device 4000 may be changed based on the aggregate information received from the RFID system 9000 and other information from system 8000. In some aspects, the indication may be that the air circuit 4170 is not coupled to the patient interface 3000 or is improperly coupled to the patient interface 3000.

In some aspects, the system 8000 may indicate to a patient when a patient interface or cushion has been worn long enough or is an old enough production batch identification number or a serial identification number that performance may be impacted and a new patient interface or cushion should be used. In some aspects, the age of the patient interface or how many times the patient interface has been used may lead to generation of an indicator that a new patient interface or new cushion should be used. The system 8000 may indicate a patient to replace the patient interface or cushion. This indication may also depend, at least in part, on the recommended usage period of a particular patient interface or cushion type, the type of therapy being administered to the patient, or one or more other factors. Indications may be generated as described above, e.g., on one or more of a display of the RPT device 4000 (e.g., the display 4294) or other component of the system 8000, to an external device, such as the remote external device 4286 or the local external device 4288 (e.g., a patient's tablet, smartphone, or computer, or a device of a healthcare provider), or to a cloud server, such as remote external communication network 4282 and/or local external communication network 4284 and/or remote external communication network 4282 (e.g., for remote monitoring, to improve patient experience, to trigger an order (such as for a new patient interface or accessory, etc.)).

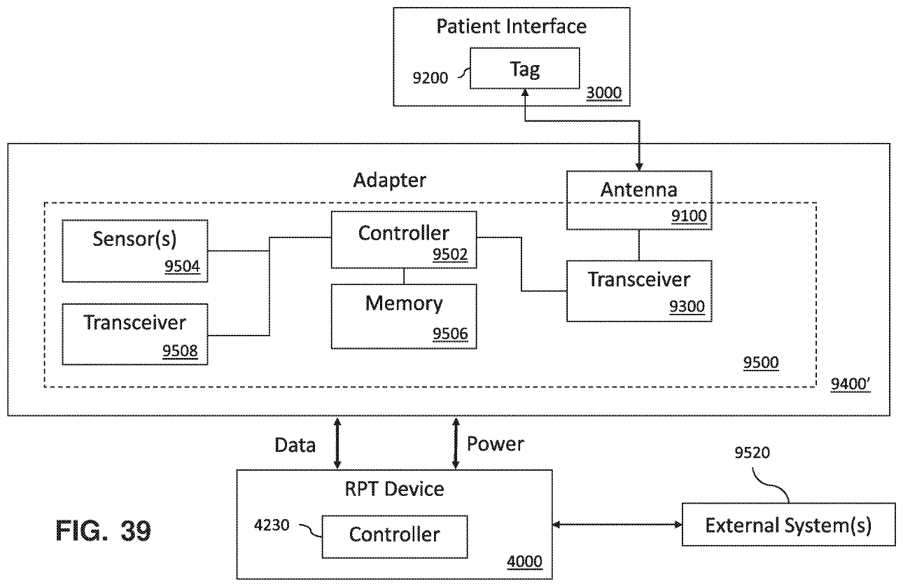

In some examples, system 8000 and/or system 8000′ may include or communicate with (e.g., over the Internet or other data communications network) one or more external system(s) 9520 as shown in FIG. 39. Illustrative examples of external system(s) include 4286 and 4288 as discussed herein. Such external computer systems may be, for example, dedicated services and/or hosted on a cloud computing platform (such as AWS, Azure, etc.). Any or all of the analysis, processing, operations, and/or actions performed by system 8000 and/or system 8000′ (discussed herein), may instead be performed by, or in conjunction with, the one or more computer systems. As an illustrative example, data may be received by RPT device 4000 that includes: 1) data from a tag included with a patient interface (e.g., identifying which type of patient inface is being used and/or other data as discussed herein) or other component of system 8000 or system 8000′, and 2) data from sensors (e.g., air flow, pressure, air leaks, humidity, VOC sensor, and the like) related to operation of system 8000 (or 8000′). The RPT device 4000 may communicate such data to the one or more computer systems for processing, analysis, or the like. One or more data messages may then be communicated from the one or more computer systems back to the RPT device 4000 to carry out, for example, changing or modifying the operation thereof. The content and/or transmission of the data messages back to the RPT device may be based on the processing and/or analysis performed by the one or more computer systems. In some examples, any or all of the analysis or processing performed by system 8000 and/or system 8000′ may be performed by a mobile device (e.g., a mobile phone or tablet) or other computing device (e.g., a desktop computer) that is configured to communicate with system 8000 and/or system 8000′ to cause such operations or actions.

The antenna 9100 and the transceiver 9300 may read a signal from the tag 9200 at least once during a therapy session. For example, the tag 9200 may be read when therapy starts, e.g., when a start button is pressed or when auto-start is initiated. In some aspects, if no accessory or the air circuit 4170 is detected as being attached to a patient interface 3000, then the RFID system 9000 may continue to read, e.g., continuously or at regular intervals. In some configurations of the technology, a signal from the tag 9200 may be read periodically, e.g., at every few seconds, minutes, or hours. In some examples of the technology, the antenna 9100 and the transceiver 9300 may read the signal from the tag 9200 according to a regular or irregular frequency. For example, the signal may be read more or less frequently at the beginning of a session or after a pre-determined amount of time. In some aspects, the frequency may increase if an unexpected reading occurs, or in the event of a failed reading. In some aspects, the type of therapy being administered or type of patient interface being used may at least in part determine the frequency with which the tag 9200 is read. In some examples, data may be acquired from tag 9200 and/or sensors based on an instruction generated or received from, for example, RPT device 4000 and/or an external computing system that is hosted in, for example, a cloud computing platform. For example, the VOC sensor discussed herein may be used to acquire such data based on an instruction received, for example, from a physician.

As described herein (e.g., see FIG. 5B), the RFID system may comprise an adapter 9400 that forms a separate and distinct component configured to be fixedly or removably coupled between the air circuit 4170 and the patient interface 3000. The adapter 9400 may comprise or contain antenna 9100, transceiver 9300 and/or one or more sensors. In such a way, the adapter 9400 may be used in conjunction with a variety of air circuits 4170 or may be used to retrofit an air circuit 4170 without an antenna, transceiver and/or one or more sensors. In some aspects, the adapter 9400 may be reusable, and the air circuit 4170 may be replaceable. In examples, the antenna 9100, transceiver 9300 and/or one or more sensors of the adapter 9400 may have any or all of the characteristics of the antenna 9100, transceiver 9300 and/or one or more sensors described in the subject application.

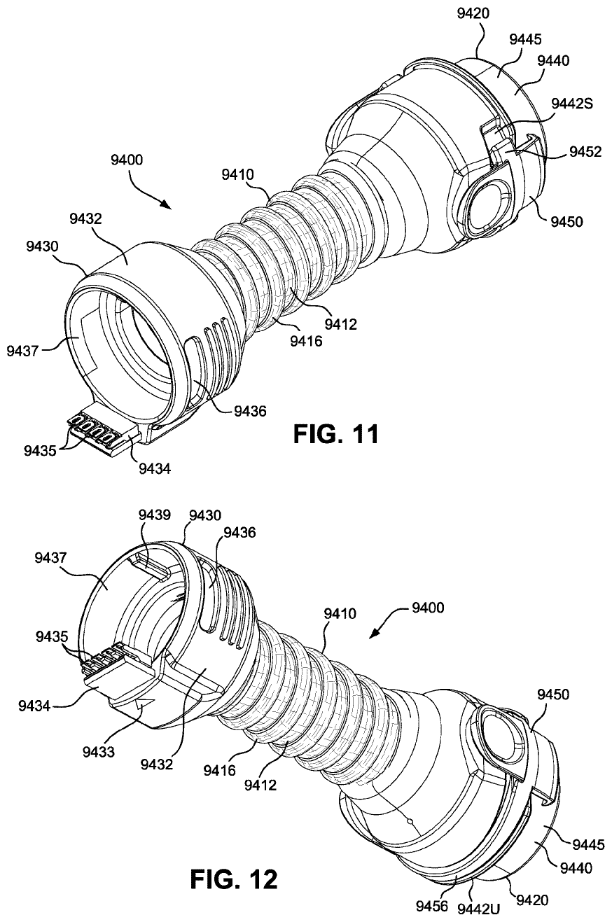

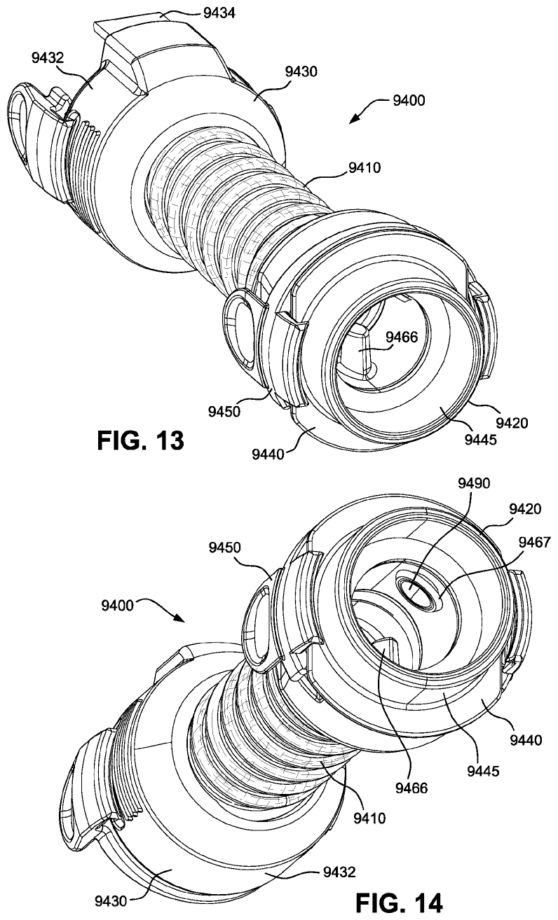

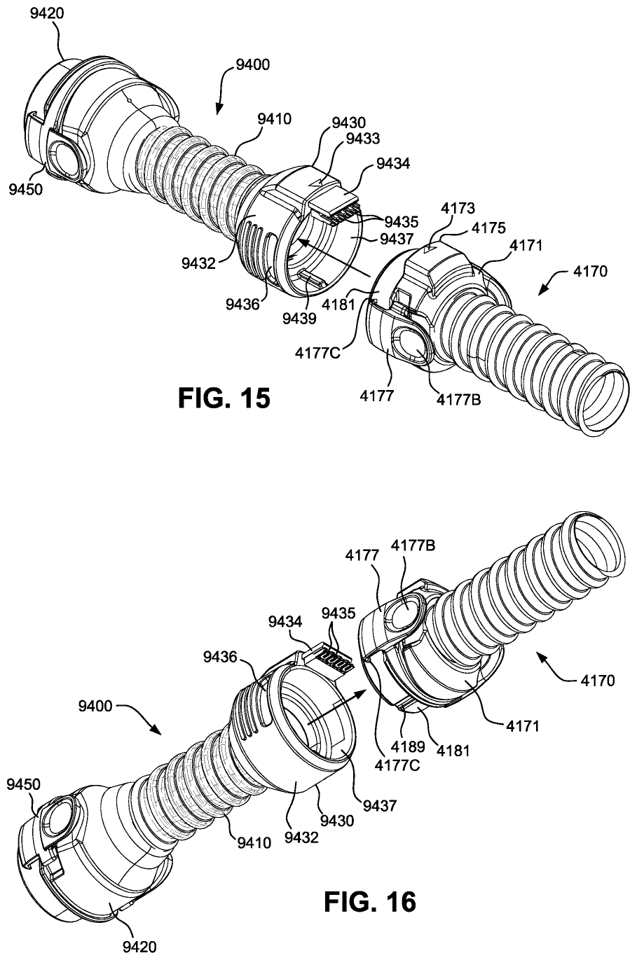

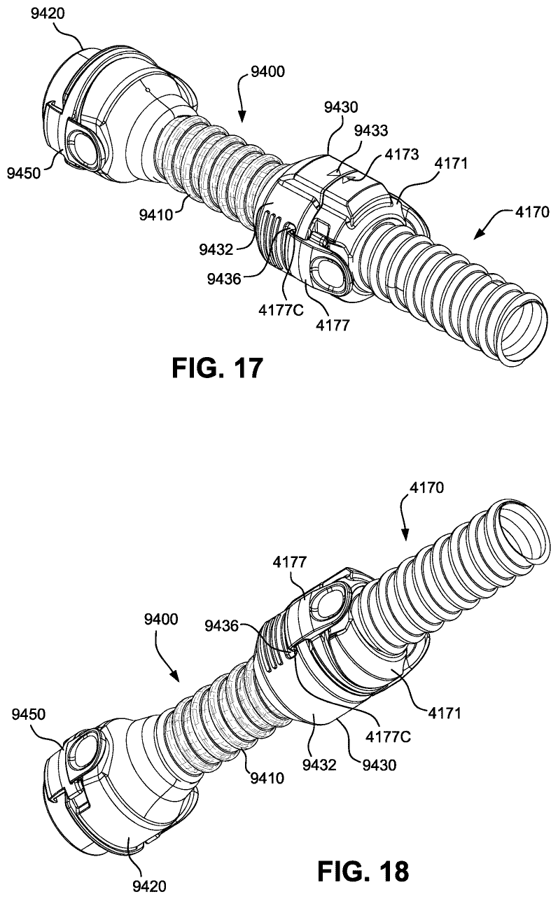

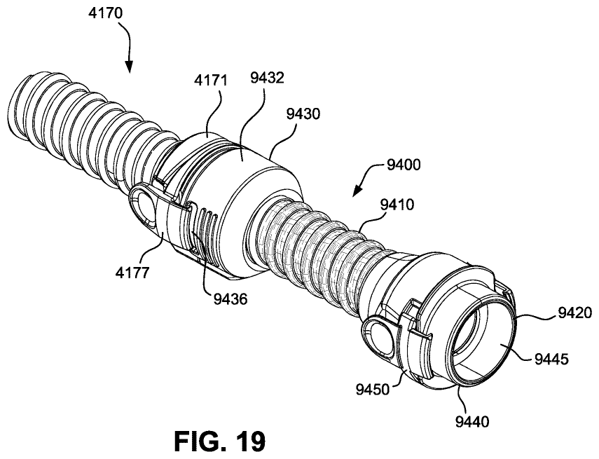

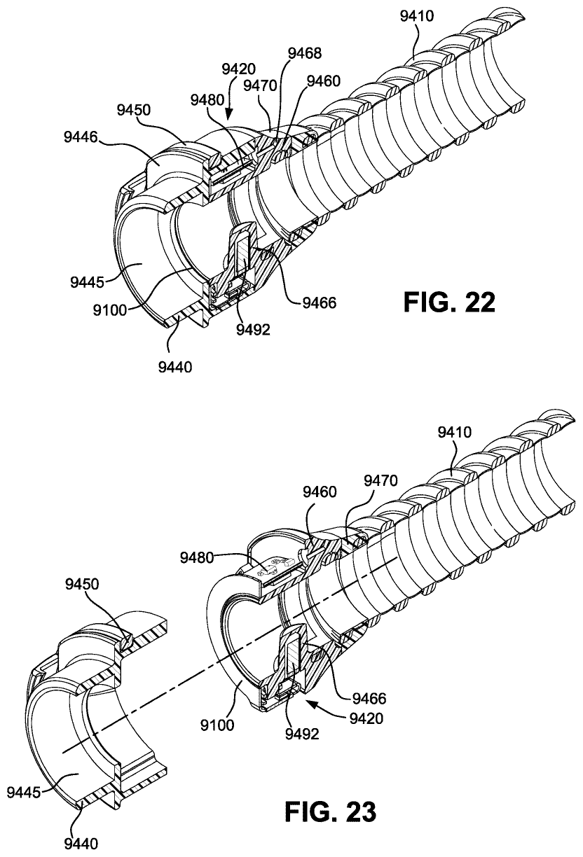

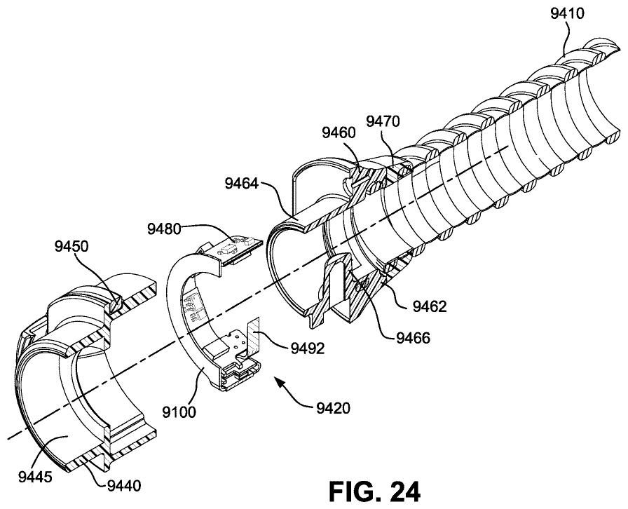

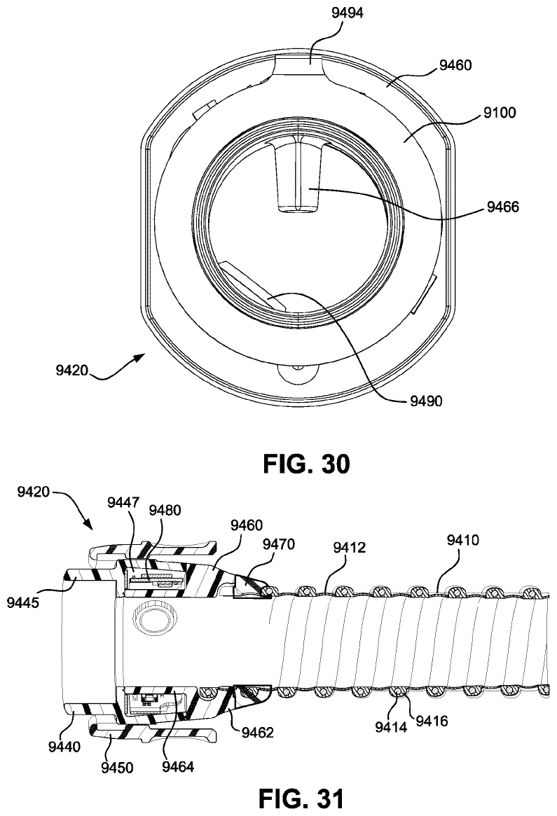

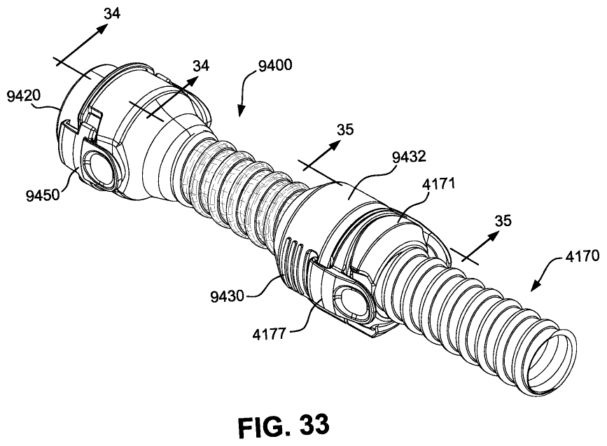

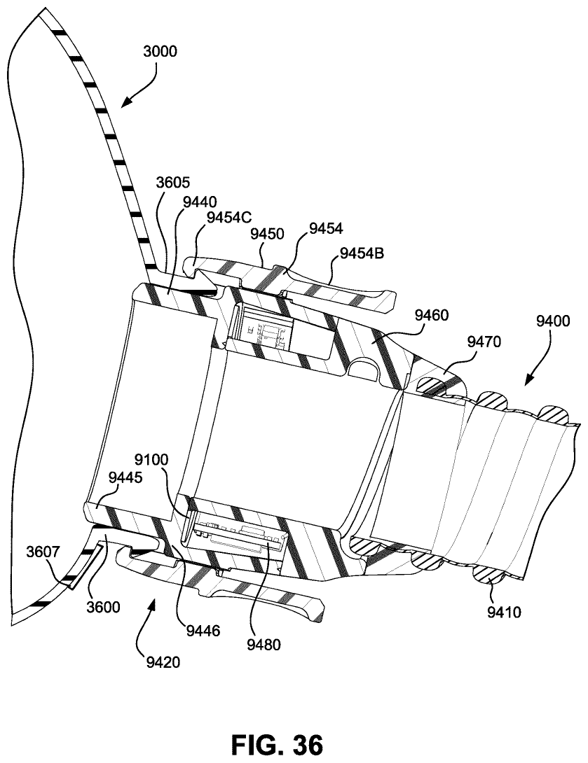

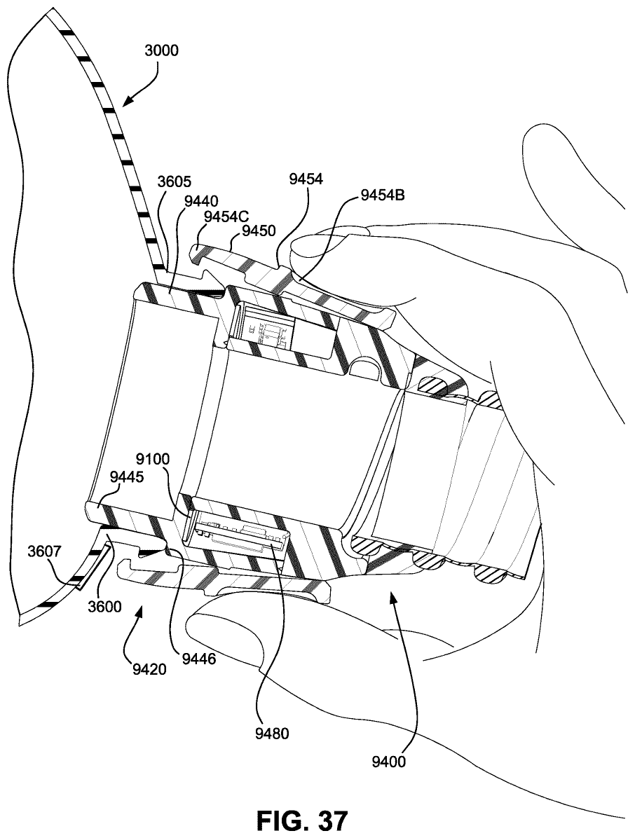

FIGS. 11-38 show an air adapter tube 9400 (also referred to as an adapter) according to an example of the present technology. As illustrated, the air adapter tube 9400 includes a tubular body 9410 (or lumen) configured to transport pressurized treatment air between a proximal end 9420 of the tubular body and a distal end 9430 of the tubular body. The proximal end 9420 is configured to connect to a patient interface 3000 (e.g., see FIGS. 36-37), and the distal end 9430 is configured for connection to an air delivery tube 4170 (e.g., see FIGS. 15-19, 33, and 35). As such, the tubular body 9410 (or lumen) is configured to permit pressurized treatment air to flow from the air delivery tube 4170, through the adapter 9400, and into the patient interface 3000.

The tubular body 9410 comprises a tube wall 9412 that forms a path for delivery of the flow of air, one or more electrical conductors 9414 (e.g., copper or aluminium wire(s)), and a helix or helical rib 9416, e.g., see FIGS. 11, 12, 20, 21, and 31.

In an example, the tubular body 9410 may comprise a flexible material, e.g., the tube wall 9410 may comprise a flexible material configured to allow the tube wall to flex or bend in use. In an example, an exterior surface of the tubular body may comprise or may be covered with a textile material, e.g., the tube wall 9410 may comprise or be covered with a textile material as shown in FIGS. 11-19. For example, the tube wall 9410 may comprise a film (e.g., comprising a polymeric material) that is covered with a textile material. However, the tube wall may comprise other materials, e.g., a polymer. In an example, the tube wall 9410 may comprise a textile material and may comprise a distinct color (e.g., blue), e.g., in order to clearly distinguish from the air delivery tube 4170 and/other tubing. The tube wall 9410 may comprise a circular cross section as illustrated, although other shapes are possible, e.g., oval. In an example, the tubular body may comprise an internal diameter between about 10-15 mm, e.g., 12 mm, so that the air adapter tube is flexible, slinky, and light to enhance user experience. In an example, the internal diameter is at least 12 mm, e.g., 12-15 mm, to reduce impedance. In an example, the tubular body may comprise a length between about 5-15 cm, e.g., 8-10 cm, 8.5-10 cm, 8.5 cm. In an example, the length is between 8.5-10 cm (e.g., at least 8.5 cm, about 8.5 cm), e.g., so that the air adapter tube will hang vertically downward from the patient interface in use, the air adaptor tube will not act like a lever arm and/or the weight of the air adaptor tube will not generate tube drag that adversely affects the seal of the patient interface.

The one or more electrical conductors 9414 are configured to carry, or transmit, an electrical signal and/or power. The one or more electrical conductors 9414 may extend throughout a length of the tubular body 9410, e.g., to provide heat to the length (partial or full) of the air adapter tube 9400 and/or to transmit electrical signal(s) and/or power from the proximal end 9420 to the distal end 9430 (e.g., from a sensor located in the proximal end 9420).

In an example, the tubular body 9410 may comprise four electrical conductors 9414, however more or less conductors are possible, e.g., two, three, or more electrical conductors. For example, the tubular body may comprise two electrical conductors 9414, i.e., one conductor for power and one conductor for data/signals. In an example, two additional wires may be provided for heat if necessary. That is, if heating of the tubular body is not provided or necessary, the tubular body may only include two wires for power and data/signals. In an example, each of the electrical conductors may comprise a wire of 30 AWG (American Wire Guage), however alternative wire gauges are possible.

In an example, the helical rib 9416 is helically wound along a length of the tube wall 9412, e.g., along the exterior surface of the tube wall. The helical rib 9416 may be integrally formed with the tube wall 9412 (e.g., of a textile or polymer material), or may be formed separately and connected thereto. The helical rib 9416 is configured to increase a rigidity of the tube wall and/or provide additional insulation. In addition, the helical rib 9416 is configured to provide sheathing for the one or more electrical conductors 9414, i.e., enclose the one or more electrical conductors so as to insulate and/or protect the electrical conductors.

The proximal end 9420 of the air adapter tube 9400 is configured to be repeatably connectable to and disconnectable from the patient interface 3000 to facilitate a releasable or separable connection between the air adapter tube 9400 and the patient interface 3000. When the air adapter tube 9400 and the patient interface 3000 are connected to one another, the proximal end 9420 is configured to form a substantially fluid-tight seal with the patient interface 3000.

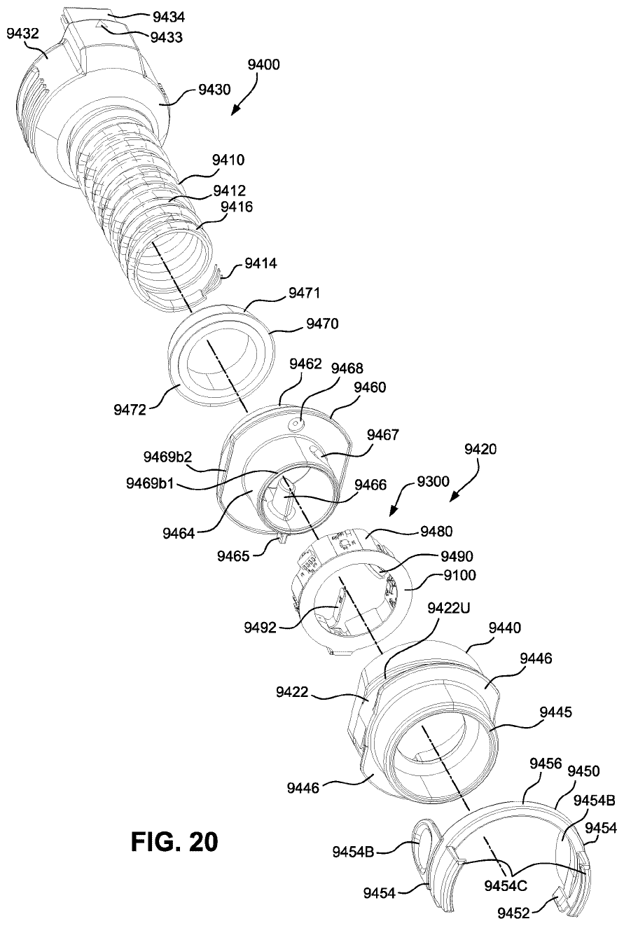

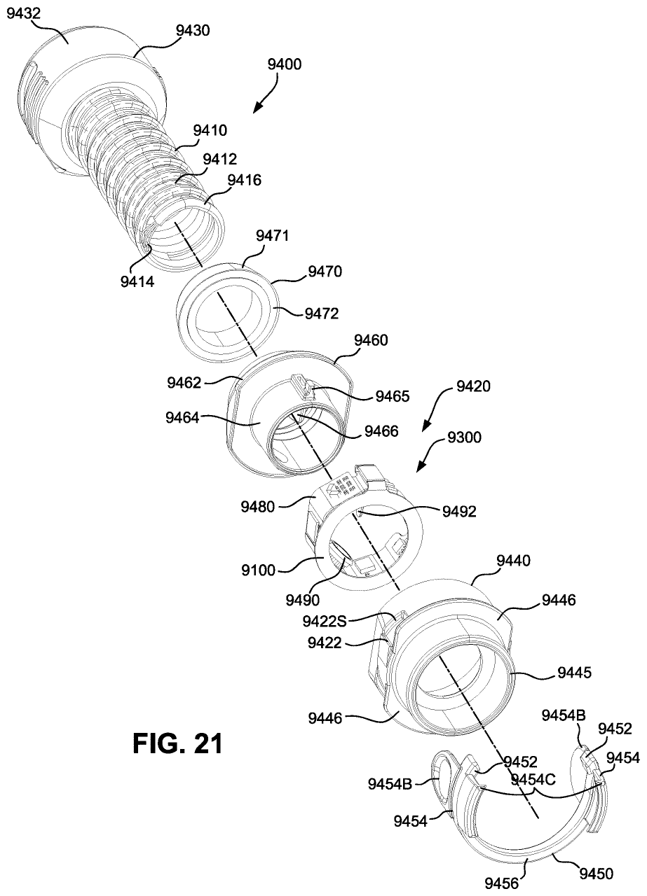

In the illustrated example, the proximal end 9420 includes a cuff portion 9440 (also referred to as a cuff front) and a mechanical connector (e.g., clip member 9450) configured to connect to the patient interface. In the illustrated example, as shown in FIGS. 20 and 21, the clip member 9450 comprises a separate and distinct structure from the cuff portion 9440, i.e., the cuff portion 9440 and the clip member 9450 comprise separately molded components that are subsequently connected to one another. In the illustrated example, the clip member 9450 is configured to provide a releasable connection, e.g., releasable snap-fit connection or separable snap joint assembly, with the cuff portion 9440.

As illustrated, the cuff portion 9440 includes one or more recesses 9442 configured to receive the clip member 9450, i.e., an upper recessed portion 9442U extending along an upper portion of the cuff portion 9440 leading to side recessed portions 9442S on respective sides of the cuff portion 9440 (e.g., see FIGS. 20-21). Each of the side recessed portions 9442S forms a distinct recess configured and arranged to interact with respective lugs 9452 of the clip member 9450 to facilitate retention of the clip member 9450 on the cuff portion 9440 (e.g., see FIG. 11). When connected, the clip member 9450 is arranged along an exterior surface of the cuff portion 9440 outside the air flow path formed by the cuff portion 9440.

The cuff portion 9440 also includes a tubular end portion 9445 configured to extend at least partially into a connection port 3600 of the patient interface 3000 so as to form a substantially fluid-tight seal with the patient interface 3000 for delivery of pressurized gas (e.g., see FIGS. 36-37). In an alternative example, the connection port 3600 may extend at least partially into the cuff portion 9440 to form a substantially fluid-tight seal.

The clip member 9450 includes a pair of resilient, quick release pinch arms or clips 9454 and a connecting portion 9456 that interconnects the pinch arms 9454. Each of the pinch arms 9454 includes a catch portion 9454C and a button or finger portion 9454B. The pinch arms 9454 are structured and arranged to provide a releasable snap-fit connection or separable snap joint assembly with the patient interface, e.g., catch portions 9454C configured to deflect and snap into a clip channel 3605 along the connection port 3600 of the patient interface 3000 (e.g., see FIG. 36). The button portions 9454B are structured and arranged to be manually pinched or squeezed to deflect the catch portions 9454C for separation or release of the catch portions from the patient interface 3000 and hence allow separation of the air adapter tube 9400 from the patient interface 3000 (e.g., see FIG. 37). In the illustrated example, each catch portion 9454C includes a lead-in angle to facilitate push-on assembly and a 900 return angle to resist or prevent pull-off disassembly, e.g., user must deflect the catch portions via the button portions to allow disconnection. Also, each pinch arm 9454 includes lug 9452 to facilitate retention of the clip member 9450 on the cuff portion 9440 (e.g., see FIG. 11).

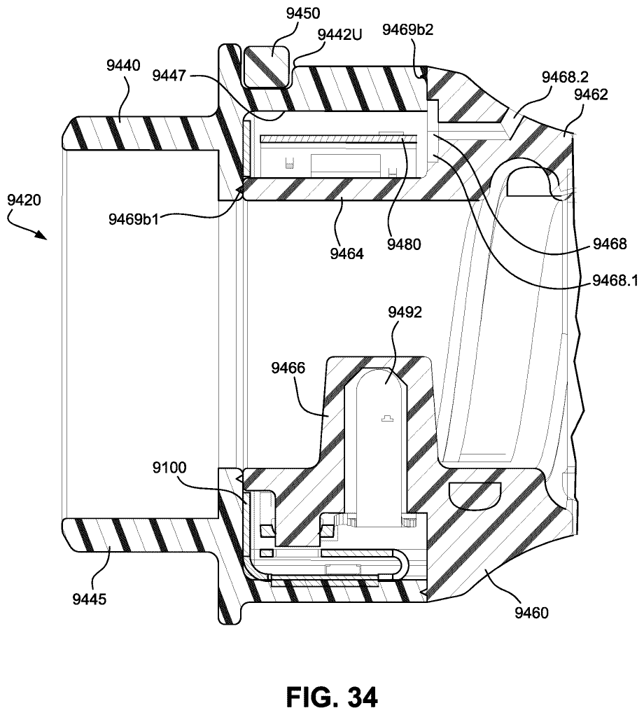

In the illustrated example, the clip member 9450 comprises an open-ended configuration with a semi-flexible and generally semi-circular connecting portion 9456 which allows the clip member 9450 to be connected to the cuff portion 9440, e.g., in a manner similar to a circlip. When connected, the connecting portion 9456 of the clip member 9450 is arranged within the upper recessed portion 9442U of the cuff portion 9440 (e.g., see FIGS. 12 and 34) and the pinch arms 9454 are arranged along respective sides of the cuff portion 9440 with the lugs 9452 fit into respective side recessed portions 9442S of the cuff portion 9440 to positively and releasably interconnect the clip member 9450 and the cuff portion 9440 in an assembled position (e.g., see FIG. 11). In an alternative example, the clip member 9450 may be non-removably or permanently connected to the cuff portion 9440. In an alternative example, the clip member and the cuff portion may be integrally formed as a one-piece construction.

In an example, the connection port 3600 of the patient interface 3000 forms a clip channel 3605 (e.g., via a flange along the connection port as shown in FIGS. 36-37) configured to matingly receive the catch portions 9454C of the clip member 9450. For example, the clip channel 3605 may be configured to receive the rib or catch of each of the catch portions 9454C to releasably retain the air adapter tube 9400 to the patient interface 3000 and form a swivel connection, e.g., allow 360° free rotation of the air adapter tube 9400 relative to the patient interface 3000 about the axis of the connection port 3600. The button portions 9454B may be manually pinched or squeezed to disengage the catch portions 9454C from the clip channel 3605 (e.g., see FIG. 37).

In the illustrated example, as shown in FIG. 36, when the air adapter tube 9400 is connected to the patient interface 3000, the tubular end portion 9445 of the cuff portion 9440 extends at least partially into a connection port 3600 so that at least a radially outwardly facing surface of the tubular end portion 9445 engages the connection port 3600 to form a substantially fluid-tight seal with the patient interface 3000 for delivery of pressurized gas. In an example, the leading end of the tubular end portion 9445 may engage a portion of the connection port 3600 form a seal with the connection port 3600.

Also, the cuff portion 9440 comprises a radially outwardly extending flange or flanges 9446 (forming at least part of the recess 9442) that acts as a stop to prevent over-insertion of the air adapter tube 9400 into the patient interface, e.g., see FIGS. 36 and 37.

In an example, the air adapter tube 9400 may provide decoupling of the air delivery tube 4170 from the patient interface 3000, e.g., to decouple tube drag on the patient interface to prevent seal instability. For example, decoupling may be provided by the pinch arms 9454 which form the swivel connection allowing 360° free rotation of the air adapter tube 9400 (and hence the air delivery tube 4170) relative to the patient interface 3000.

In an alternative example, in lieu of clip member 9450, the mechanical connector of the air adapter tube 9400 may include an iso-taper configured to connect with the patient interface with an interference or frictional fit. For example, the cuff portion 9440 of the air adapter tube may include an iso-taper configured to connect to the connection port 3600 of the patient interface 3000.

In the illustrated example, the proximal end 9420 includes one or more adapters to couple the cuff portion 9440 and support or contain antenna 9100, transceiver 9300 and/or one or more sensors.

As shown in FIGS. 20 and 21, the proximal end 9420 includes a first adapter element 9460 (also referred to as a preblock or preblock 1) and a second adapter element 9470 (also referred to as adapter element or preblock 2).

The first adapter element 9460 includes a body portion 9462 and a tubular projection 9464 extending proximally from the body portion 9462. The body portion 9462 is configured to connect to the second adapter element 9470, and the tubular projection 9464 is configured to support and/or retain the antenna 9100, transceiver 9300 and/or one or more sensors.

The second adapter element 9470 is configured and arranged to interconnect the first adapter element 9460 and the tube wall 9410 of the tubular body. For example, the second adapter element 9470 includes a first end 9471 that is connected to the tube wall 9410 and the helical rib 9416, e.g., second adapter element 9470 overmolded to the tube wall 9410 (e.g., film) and/or the helical rib 9416 so that the second adapter element 9470 connects and seals with the tube wall 9410 and/or the helical rib 9416. The second adapter element 9470 includes a second end 9472 (opposite to the first end 9471) that is connected to the body portion 9462 of the first adapter element 9460, e.g., second adapter element 9470 overmolded to the first adapter element 9460. However, it should be appreciated that the first adapter element 9460, the second adapter elements 9470, the tube wall 9410 and the helical rib 9416 may be formed and connected to one another in other suitable manners, e.g., overmolded connection, mechanical connection, integrally formed in one-piece.

In the illustrated example, the transceiver 9300 is provided on a printed circuit board (PCB) 9500. An example of PCB 9500 is a flexible printed circuit (FPC) or flexible circuit board 9480. Similar to examples described herein (e.g., see FIGS. 6A and 6B), the FPC 9480 comprises one or more bendable or flexible materials configured and arranged to allow the FPC 9480 to bend or flex around the tubular projection 9464 of the first adapter element 9460, e.g., see FIGS. 20-24 and 27-32.

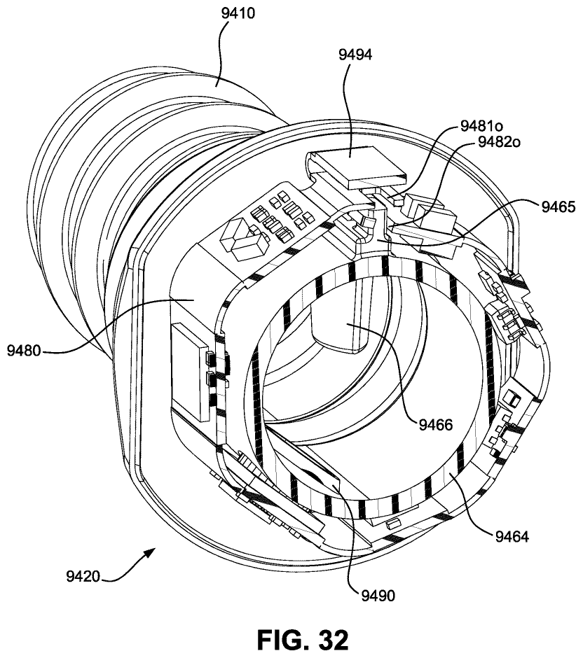

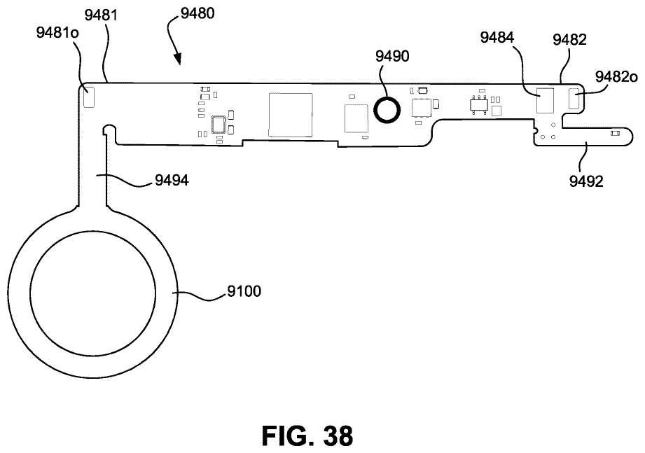

As illustrated, the FPC 9480 include a first end 9481 and a second end 9482 opposite to the first end 9481. Each of the first end 9481 and the second end 9482 includes a respective opening 9481o, 9482o (e.g., see FIGS. 32 and 38). The FPC 9480 is configured to be bent or flexed around an outer cylindrical surface of the tubular projection 9464, and first and second ends 9481, 9482 at least partially overlap to allow a retaining member 9465 (extending radially outwardly from the tubular projection 9464) to protrude through one or both respective openings 9481o, 9482o so as to support, retain, align, and orient the FPC 9480 in position on the first adapter element 9460, e.g., see FIGS. 20, 21, and 32. That is, the first adapter element 9460 includes at least one feature (e.g., fastener (e.g., retaining member 9465) and/or adhesive (e.g., silicone)) configured to support and retain the FPC 9480.

The tubular projection 9464 of the first adapter element 9460 also includes at least one port 9467, which allows at least one sensor (e.g., pressure sensor 9490) provided to the FPC 9480 to protrude radially inwards into and/or through the port 9467. That is, the port 9467 allows the pressure sensor 9490 provided to the FPC 9480 to communicate with the flow of air passing through the air adapter tube 9400. In an example, silicone may be provided to seal the sensor 9490 within the port 9467.

A sensor support 9466 is also provided to the first adapter element 9460. The sensor support 9466 extends radially inwards from the tubular projection 9464, and includes an interior recess configured to receive a sensor (e.g., a thermistor or gas temperature sensor 9492) provided to the FPC 9480. In the illustrated example, the sensor support 9466 and respective temperature sensor 9492 (e.g., thermistor) communicate with the flow of air passing through the air adapter tube 9400, e.g., to measure the temperature of the air flow. In an example, the sensor support 9466 may be overmolded to the temperature sensor 9492 of the FPC 9480.

In an example, the pressure and temperature sensed by the sensors 9490, 9492 may be provided to FPC 9480. For example, an electronic circuit included with the air adapter tube 9400 may be used to read data from the sensors. The data then be converted into a format by the electronic circuit and communicated to a controller (e.g., via the electrical conductors 9414 (wires) and/or the antenna 9100. In an example, the sensors (e.g., including 9490, 9492) included in air adapter tube 9400 may be polled or otherwise communicate data therefore at the same or different rates. In an example, any or all of the sensors of air adapter tube 9400 may be polled continuously or at a lesser (or on demand) frequency. As an illustrative example, pressure sensor 9490 may be polled at a rate of no less than 500 Hz. The rate of polling for a given sensor may be based on how the data is used. In an example, polling for some of the sensors included with air adapter tube 9400 may be at a lower frequency. For example, data from an inertial sensor may be polled at, as an illustrative example, every 1 minute or the like.

Data from the pressure and/or temperature sensed by the sensors 9490, 9492 (or other sensors discussed herein) may be communicated to another system, such as an external computer system (e.g., a cloud computing platform, a mobile device, etc.) and then processed or analysed thereon. The results of such processing or analysis may then be communicated back to the RPT device 4000 and/or the adapter for use (e.g., to control parameters associated with therapy delivered to a patient).

In an example, pressure data from the pressure sensor 9490 (or data based thereon) may be communicated back to an RPT device or flow generator and be used to control pressure that is being supplied to a patient. In an example, data acquired by the pressure sensor 9490 may be used in real-time control of generated pressure. In an example, pressure values supplied by pressure sensor 9490 may be used in the same or similar manner to that of pressure sensor 4272 discussed herein. In any example, the pressure values supplied by pressure sensor 9490 may be used to calculate, Pm, or used in connection with the interface pressure estimation algorithm 4312 as discussed herein. In an example, data from pressure sensor 9490 may be used to compensate for pneumatic errors within an air circuit (e.g., air circuit 4170).

The cuff portion 9440 is configured to connect to the first adapter element 9460. When connected, the cuff portion 9440 and the first adapter element 9460 form an annular space 9447 therebetween configured to receive the FPC 9480 supporting the pressure sensor 9490 and the temperature sensor 9492, e.g., see FIGS. 31 and 34.

In an example, the cuff portion 9440 may be secured to the first adapter element 9460 by ultrasonic welding. In an example, the first adapter element 9460 may include one or more energy directors (e.g., along the free end of the tubular projection 9464 and/or along the outer perimeter of the first adapter element 9460) to facilitate the ultrasonic welding process. For example, the energy director may include a raised bead (e.g., a raised bead 9469b1 along the free end of the tubular projection 9464 and a raised bead 9469b2 along the outer perimeter of the first adapter element 9460 as shown in FIGS. 20, 27, and 34) to expedite formation of the joint between the cuff portion 9440 and the first adapter element 9460 during the ultrasonic welding process.

In an example, at least one vent hole 9468 may be provided along a perimeter of the first adapter element 9460, e.g., see FIGS. 20, 22, 28, and 34. As illustrated the vent hole 9468 includes a first end 9468.1 in communication with the annular space 9447 receiving the FPC 9480 and second end 9468.2 in communication with an exterior of the air adapter tube, e.g., ambient. In an example, the first end 9468.1 may include a larger diameter than the second end 9468.2, and the vent hole 9468 may include a non-linear path from the first end 9468.1 to the second end 9468.2. In an example, a water resistant pressure balancing membrane may be provided to the vent hole 9468, e.g., over the first end 9468.1. The water resistant pressure balancing membrane may be configured to adjust differential pressure at the proximal end 9420 (between annular space 9447 and ambient) and prevent damage while resisting water/dust from entering the annular space with the FPC 9480. That is, the water resistant pressure balancing membrane ensures a balance of pressure between the annular space 9447 and ambient so that the FPC 9480 and its sensors (e.g., pressure sensor 9490) can function reliably.

The distal end 9430 of the air adapter tube 9400 is configured to be repeatably connectable to and disconnectable from the air delivery tube 4170 to facilitate a releasable or separable connection between the air adapter tube 9400 and the air delivery tube 4170, e.g., see FIGS. 15-19. When the air adapter tube 9400 and the air delivery tube 4170 are connected to one another, the distal end 9430 is configured to form a substantially fluid-tight seal with the air delivery tube 4170.

In the illustrated example, the distal end 9430 includes a cuff portion 9432 and an electrical connector 9434, e.g., provided to a superior side of the cuff portion 9432. The electrical connector 9434 includes a lead frame with one or more electrical contacts 9435, e.g., four contacts, configured to transmit power and/or signals. The electrical contacts 9435 are electrically connected to respective electrical conductors 9414 (wires) extending along the tubular body 9410. The electrical connector 9434 is configured to electrically connect to a corresponding electrical connector 4175 of the air delivery tube 4170 (e.g., a heated air delivery tube).

The distal end 9430 of the air adapter tube 9400 includes a mechanical connector (e.g., recessed side portions 9436) configured to mechanically connect to a corresponding mechanical connector (e.g., a pair of resilient, quick release pinch arms or clips 4177) of the air delivery tube 4170. Each of the pinch arms 4177 includes a catch portion 4177C and a button or finger portion 4177B. The pinch arms 4177 are structured and arranged to provide a releasable snap-fit connection or separable snap joint assembly with the distal end 9430, e.g., catch portions 4177C configured to deflect and snap into a respective one of the recessed side portions 9436 as shown in FIGS. 15-19. The button portions 4177B are structured and arranged to be manually pinched or squeezed to deflect the catch portions 4177C for separation or release of the catch portions from the distal end 9430 and hence allow separation of the air delivery tube 4170 from the air adapter tube 9400.

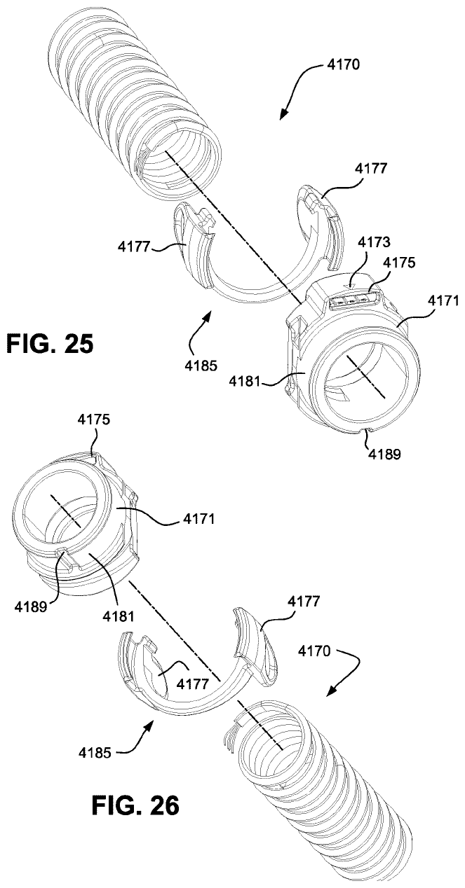

As shown in FIGS. 25 and 26, the mechanical connector (e.g., pinch arms 4177) of the air delivery tube 4170 may be configured as a separate and distinct clip member 4185 configured to provide a releasable connection with the cuff portion 4171 of the air delivery tube 4170, e.g., similar to clip member 9450 described herein. For example, the clip member 4185 may be arranged within a recess of the cuff portion 4171 with lugs of the clip member 4185 fit into respective side recessed portions to positively and releasably interconnect the clip member 4185 and the cuff portion 4171 in an assembled position. In an alternative example, the clip member and the cuff portion may be integrally formed as a one-piece construction.

However, it should be appreciated that the distal end 9430 and the air delivery tube 4170 may be connected to the air delivery tube 4170 in other suitable manners. In an alternative example, the distal end 9430 and the cuff portion 4171 may include an iso-taper configured to connect to one another in an interference or frictional fit.

In the illustrated example, the air delivery tube 4170 does not include any sensors (e.g., temperature sensor, pressure sensor, etc.). In an alternative example, the air delivery tube 4170 may include at least one sensor (e.g., temperature sensor), e.g., at least one sensor not provided to the air adapter tube 9400.

When the air delivery tube 4170 is connected to the air adapter tube 9400, the cuff portion 4171 includes a tubular end portion 4181 configured to extend at least partially into opening 9437 of the cuff portion 9432 so that at least a radially outwardly facing surface of the tubular end portion 4181 engages the cuff portion 9432 to form a substantially fluid-tight seal with the air adapter tube 9400 for delivery of pressurized gas, e.g., see FIGS. 15, 16, and 35. In an example, the leading end of the tubular end portion 4181 may engage a portion of the cuff portion 9432 to form a seal with the cuff portion 9432.

In the illustrated example, as shown in FIGS. 12, 15, 16, 25, and 26, the cuff portion 9432 includes a radially inwardly extending projection 9439 configured to engage with a corresponding recess 4189 in the tubular end portion 4181 when the air delivery tube 4170 is connected to the air adapter tube 9400, e.g., to facilitate alignment, prevent relative rotation.

In the illustrated example, the proximal end 9420 of the air adapter tube 9400 and the air delivery tube 4170 include similar mechanical and pneumatic connectors (e.g., a pair of resilient, quick release pinch arms 9454, 4177 and tubular end portion 9445, 4181) configured to allow both the air adapter tube 9400 and the air delivery tube 4170 to form a mechanical and pneumatic connection with the patient interface 3000, e.g., air adapter tube 9400 and the air delivery tube 4170 may connect to the patient interface 3000 independently.

In an example, as shown in FIGS. 12, 15, 17, 20, and 25, the cuff portion 9432 of the distal end 9430 may include an indicator or guide (e.g., alignment arrow 9433) configured to align with a corresponding indicator or guide (e.g., alignment arrow 4173) provided to the cuff portion 4171 of the air delivery tube 4170, e.g., to facilitate and confirm correct orientation, alignment, and connection of the air adapter tube 9400 with the air delivery tube 4170.

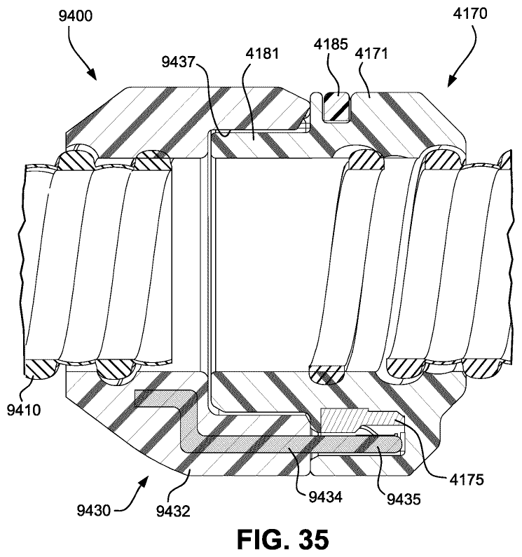

When the air adapter tube 9400 is connected to the air delivery tube 4170, the electrical contacts 9435 of the air adapter tube 9400 are arranged to engage with respective contacts provided to the electrical connector 4175 of the air delivery tube 4170 to form electrical and control signal connections with the air delivery tube 4170, e.g., see FIG. 35. In the illustrated example, the lead frame with electrical contacts 9435 on the air adapter tube 9400 are arranged as a male connector configured to form the electrical and signal connections when inserted into engagement with the electrical connector 4175 arranged as a female connector on the air delivery tube 4170, i.e., straight or direct plug-in connection. The electrical connector 4175 electrically connects its contacts to respective wires running along the air delivery tube 4170. In an example, the electrical connection may be facilitated by using, for example, serial communication, such as RS485. However, it should be appreciated that the electrical contacts may have alternative configurations and arrangements, e.g., depending on the interface arrangement or connection mechanism provided between the air adapter tube and the air delivery tube.

The electrical conductors 9414 (wires) extending along the tubular body electrically connect the electrical connector 9434 to the transceiver (e.g., FPC 9480) and/or one or more sensors at the proximal end 9420. Accordingly, electrical signal(s) (e.g., from one or more sensors) may be transmitted from the proximal end 9420 to the distal end 9430, from the distal end 9430 to the air delivery tube 4170, and from the air delivery tube 4170 to the RPT device or flow generator (e.g., RPT device or flow generator may be configured to be controlled based on output from the one or more sensors).

As noted herein, the air adapter tube 9400 includes at least one sensor supported at the proximal end 9420 by the FPC 9480, e.g., pressure sensor 9490, temperature sensor 9492. In examples, the at least one sensor may include pressure sensor 9490 and/or temperature sensor 9492 as described herein, and may also include one or more additional sensors, e.g., a flow rate sensor, a humidity sensor, and/or an inertial sensor. Inertial sensors may include, for example, accelerometers, gyro sensors, magnetometers, and the like.

The at least one sensor may produce one or more output signals which may be communicated to a controller for diagnosis and/or treatment of a respiratory disorder. For example, the pressure sensor 9490 may be configured to generate a pressure signal of air passing at the proximal end 9420 for diagnosis and/or treatment of a respiratory disorder. The temperature sensor 9492 may be configured to generate a signal indicative of air temperature at the proximal end 9420. The accelerometer sensor may be configured to generate a signal indicative of patient movement and/or sleeping position of a patient, which data may be used to allow personalization of a patient interface based on detected sleep patterns.

In an example, a sensor may be provided to the FPC 9480 that is configured to generate a signal indicative of the breath of and/or a biomarker of gas exhaled by the patient, e.g., a CO₂ sensor, a biomarker sensor.

For example, the proximal end 9420 may include a CO₂ sensor configured to detect CO₂ buildup, re-breathed CO₂ levels and/or breathing comfort. In an example, the CO₂ sensor is arranged in the patient interface between the patient and the vent on the patient interface, i.e., in a position to facilitate detection of gas exhaled by the patient. In an example, the sensor or the vent may be moved or repositioned so that CO₂ sensor is arranged upstream of the vent, i.e., to allow the sensor to detect the exhaled gas before exiting the patient interface via the vent. In an example, data from a CO₂ sensor may be communicated to the RPT device and used to control (e.g., dynamically), for example, a pressure that is supplied to the patient. As an example, if a CO₂ reading is high (e.g., above a threshold amount), then the pressure supplied from the RPT device may be increased. The resulting increased pressure (e.g., and the corresponding flow) may act to decrease the CO₂ levels for a patient.

In an example, the CO₂ sensor may be configured to detect end-tidal CO₂, which may be an indicator of patient health. For example, a low end-tidal CO₂ reading may indicate poor perfusion, hypovolemia, or sepsis, whereas a high end-tidal CO₂ reading may indicate airway narrowing, airway obstruction, or respiratory distress. An end-tidal CO₂ reading may also help the healthcare provider determine if the patient is being ventilated adequately. In an example, the end-tidal CO₂ reading may be collected within the air adapter tube, i.e., not necessary to arrange CO₂ sensor within the patient interface.

In an example, the proximal end 9420 may include a VOC (volatile organic compounds) sensor configured to detect biomarkers within the patient's breath. For example, the VOC sensor may be configured to detect asthma, diabetes, lung cancer, alcohol, and other health conditions.

In an example, the CO₂ sensor and/or the VOC sensor may be configured to conduct readings before therapy, i.e., CO₂ sensor and/or the VOC sensor could conduct pre-therapy analysis before pressurized treatment air is delivered by the RPT device or flow generator. For example, the patient may don the patient interface (which is operatively connected to the RPT device via the air adapter tube 9400) for one or more breathing cycles (e.g., 1-10 breaths) before therapy is started, which allows the CO₂ sensor and/or the VOC sensor provided to the air adapter tube 9400 to conduct a reading for analysis before the patient starts therapy. After one or more breathing cycles, the RPT device may be activated to deliver pressurized breathable air for treatment. In an example, the readings from the CO₂ sensor and/or the VOC sensor may be provided to the patient in real-time, or may be provided to a health professional for analysis before communication to the patient. In this example, the patient interface is being worn and therapy is delayed for one or more breathing cycles to allow completion of sensing by one or more sensors (e.g., CO₂ sensor and/or the VOC sensor).

In an example, reading from the CO₂ sensor and/or the VOC sensor may be performed automatically at the beginning of therapy, prior to beginning therapy, and/or prior to air flow being supplied to the patient. In certain examples, a patient donning the patient interface may be detected automatically (e.g., based on a reading from an accelerometer or the like) and then used to trigger (in whole or in part) a CO₂ sensor and/or the VOC sensor to obtain data related patient breathing. In an example, readings from the CO₂ sensor and/or the VOC sensor may be obtained as part of a startup process for the RPT device. The startup process may include additional aspects, such as checking the type of patient interface (via the tag as discussed herein), validating firmware of the RPT device, and/or the parameters to be used in the to be provided therapy. Accordingly, in certain examples, the acquisition of data from the CO₂ sensor and/or the VOC sensor may be performed without having to expressly prompt, for example, the patient to perform such readings.

In an example, the air adapter tube 9400 may include one or more additional features to enhance control and/or sensing capabilities. For example, the air adapter tube 9400 may include an on/off button to control power and/or power may be controlled via haptics based on an accelerometer sensor. The air adapter tube 9400 may include a tube temperature up/down button to control air temperature of delivered air. The air adapter tube 9400 may include a tube humidity up/down button to control air humidity of delivered air. The air adapter tube 9400 may include an optical or color sensor to detect one or more colors (e.g., ring of colors or greyscale gradient) on a portion of the patient interface or elbow, e.g., to detect patient movement or sleeping position. The air adapter tube 9400 may include a light sensor to project light and encourage paced breathing. The air adapter tube 9400 may include a microphone, e.g., to detect snoring, mouth leak, voice commands.

In an example, venting may be moved from the patient interface and into the air adapter tube (e.g., within the proximal end 9420 adjacent the patient interface), which venting on the air adapter tube could be active and controlled (e.g., electric and/or pneumatic).

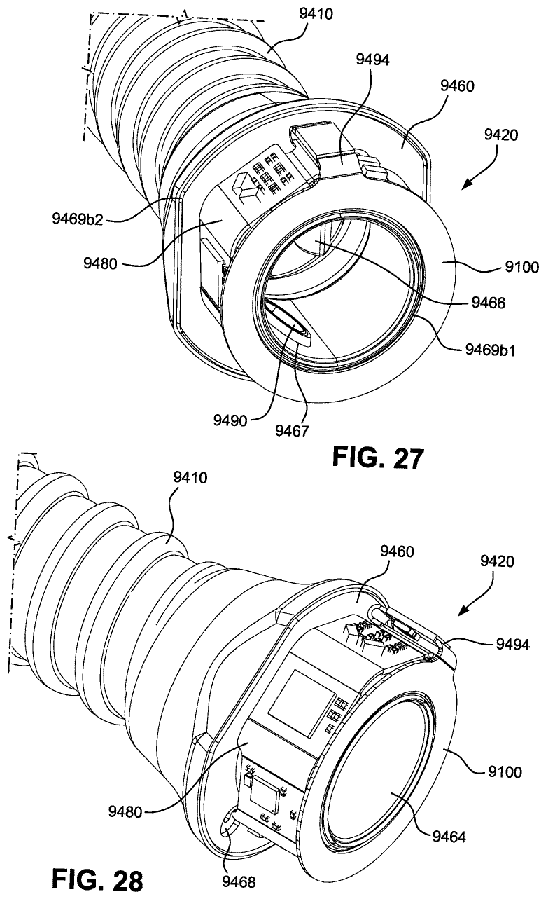

In an example, the FPC 9480 includes an antenna 9100, e.g., a near field communication (NFC) antenna. In an example, the antenna 9100 is configured to wirelessly transmit collected sensor data to an external device, including a smart phone or a flow generator. Similar to examples described herein, the antenna 9100 is configured to face proximally and lie in a plane that is generally perpendicular to an axis of the proximal end 9420. The antenna 9100 forms an opening that surrounds or encircles the lumen of the air adapter tube. The antenna 9100 is fixed and electrically coupled to the FPC 9480 by bridge 9494, and the bridge 9494 includes one or more flexible portions to allow the antenna to be bent (e.g., about 90 degrees) relative to the FPC 9480, e.g., see FIGS. 27-29 and 38. The antenna 9100 may have any or all of the characteristics of the antennas described herein.

Because the antenna 9100 is disposed in the proximal end 9420 of the air adapter tube 9400, the antenna 9100 is configured to be positioned near the connection port 3600 of the patient interface 3000 when the air adapter tube 9400 is coupled to the patient interface 3000, e.g., see FIGS. 36-37. This arrangement allows the antenna 9100 to receive data transmitted from a tag (which may be the same or similar to tag 9200, e.g., an RFID tag, an NFC tag, etc.) provided on the patient interface 3000. FIGS. 36 and 37 show an example of where tag 3607 may be located. Tag 3607 may be the same or similar to tag 9200 and used in a manner as described herein, e.g., identifying data of the patient interface and/or the patient.

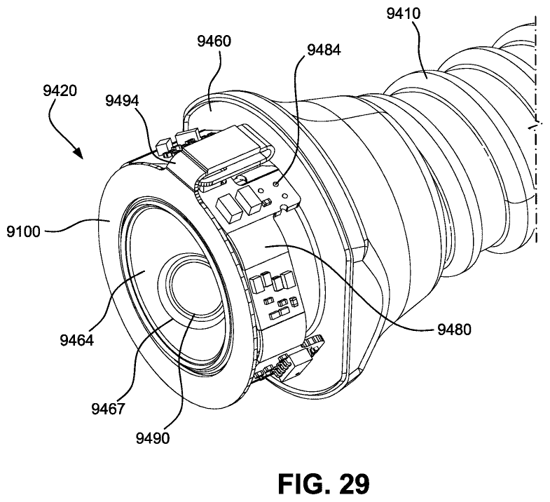

In the illustrated example, the FPC 9480 includes at least one wire connection point that connects the FPC 9480 and antenna 9100 to at least one wire (e.g., electrical conductor 9414) extending along and from the tubular body 9410. For example, as shown in FIGS. 29 and 38, the FPC 9480 may include a joint area 9484 configured to allow the FPC 9480 to form a joint (e.g., solder joint, welding joint) with each of the electrical conductors 9414 (e.g., 4 wires).

In the illustrated example, the FPC 9480 includes linear sections to support electronic components and at least one flexible bent portion between the linear sections to allow the FPC 9480 to be bent or flexed around the first adapter element 9460. In an example, the electrical components may be provided to one or both sides of the FPC. In an example, the electronic components may include the pressure sensor 9490, the temperature sensor 9492 and/or an accelerometer sensor configured to generate a signal indicative of a patient's sleeping position. In an example, the electronic components may include one more sensors configured to generate a signal representative of a breath and/or a biomarker indicative of health of a patient.

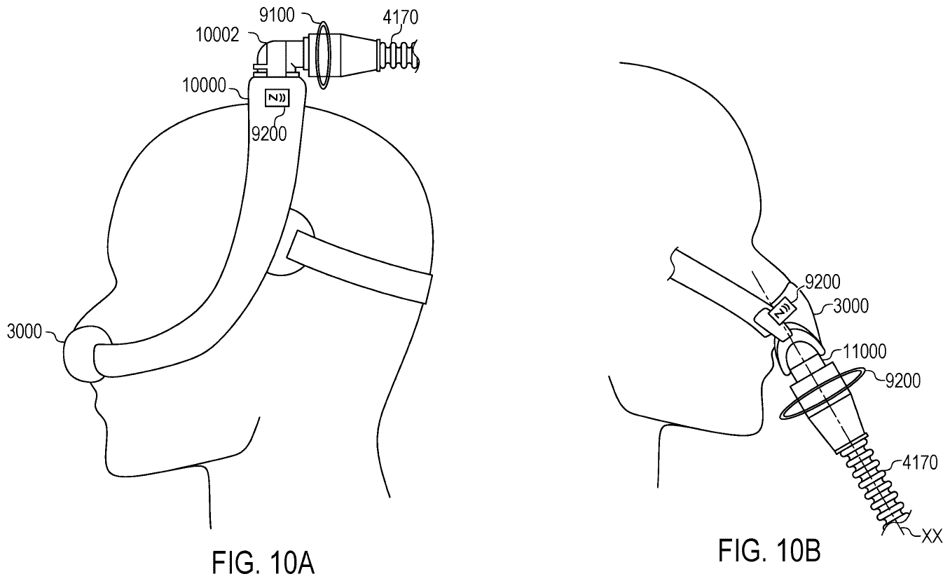

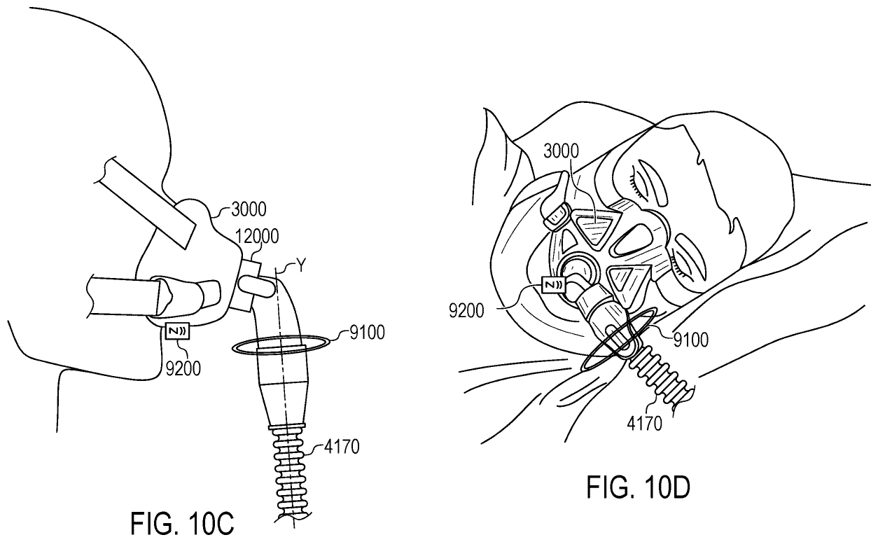

In the illustrated example, e.g., see FIGS. 36 and 37, the air adapter tube 9400 is connected directly to the patient interface 3000 (i.e., without an elbow or additional connector between the air adapter tube and the patient interface), which allows the one or more sensors provided to the FPC 9480 (as well as the antenna) to be in close proximity to the patient interface and breathing chamber thereof for collecting data. However, it should be appreciated that the air adapter tube 9400 may be arranged along air delivery path between the patient interface and the RPT device in other suitable manners. For example, the air adapter tube 9400 may be configured to connect to tube-up patient interfaces and tube-down patient interfaces according to any of the examples described herein. For example, the air adapter tube 9400 may be configured to connect between the patient interface 3000 and the air delivery tube 4170 (e.g., via an elbow) in the tube-down examples shown in FIGS. 10B to 10D, and the air adapter tube 9400 may be configured to connect between the headgear tubing and the air delivery tube 4170 (e.g., via an elbow) in the tube-up example shown in FIG. 10A.

FIG. 39 is a block diagram showing the adapter 9400′, RPT device 4000, a patient interface 3000, and one or more external systems 9520 all of which may be in communication with each other using wired and/or wireless communication. Adapter 9400′ may be the same or similar to adapter 9400 discussed herein. Aspects of adapter 9400 may be similarly applied to adapter 9400′ and aspects of adapter 9400′ may be similarly applied to adapter 9400.

Adapter 9400′ includes PCB 9500, which may be flexible in certain examples. Adapter 9400′ includes an antenna 9100 coupled to a transceiver 9300 that is configured to read data from tag 9200 of the patient interface 3000. The antenna 9100 may be electrically connected to the transceiver 9300 to thereby emit radio waves and receive signals from, for example, tag 9200.

Adapter 9400′ also includes one or more sensors 9504, memory 9506, transceiver 9508, a controller 9502. Sensor(s) 9504 may include any or all of the sensors discussed herein. An example of sensor(s) 9504 include pressure sensor 9490 and temperature sensor 9492. Memory 9506 is provided for local storage (e.g., on the adapter 9400′) of data received from sensors 9504, transceiver 9300, and the like. Memory 9506 may also store program instructions that can cause the controller 9502 to perform operations. In some examples, the memory may be part of the controller 9506 (e.g., as a system-on-chip (SoC)). In any example, adapter 9400′ also includes a power management system that is configured to supply an appropriate level of power to the one or more sensors 9504, memory 9506, transceiver 9508, the controller 9502, and transceiver 9300.

Transceiver 9508 is included with adapter 9400′ to provide data communication functionality for communicating data between the adapter 9400′ and RPT device 4000 (or other destination as required). Transceiver 9508 may provide wired, wireless, or both wired and wireless communication. In some examples, transceiver 9508 may be provided as part of the same circuitry as transceiver 9300. Accordingly, for example, adapter 9400′ may include a first transceiver, which is wireless, that is used to communicate tag 9200 and a second transceiver, which is wired, that is used to communicate to, for example, RPT device 4000 using a physical link (e.g., a wire).

In an example, transceiver 9508 provides a data link as noted above and also provides power to the power management system of adapter 9400′. The power may be provided from the RPT device (e.g., as discussed herein) or may be provided via a battery or other power source.

Adapter 9400′ also includes controller 9502. Controller 9502 may include, or be, a hardware processor that is configured to execute computer executable instructions to perform one or more operations. For example, the controller 9502 may control the sensors 9504 and/or the transceiver 9300 to acquire data (e.g., for a physical phenomenon or the data from tag 9200). In some instances, the controller 9502 control when data is communicated to RPT device 4000 (e.g., via a corresponding data link) and/or when data is acquired via the sensors 9504 and/or transceiver 9300. Controller 9502 may include a hardware processor (e.g., an ARM microprocessor), memory (e.g., for both program and data), and I/O on a single integrated circuit. An illustrative example of a controller that may be used in an illustrative example for adapter 9400′ is the STM32G071EBY6TR microcontroller.

In some examples, the controller 9502 may be used to determine when the patient interface is worn or being used. This may be determined by, for example, interpreting data from an acceleration sensor to determine when a patient is wearing patient interface 3000. Based on such a determination, the controller may activate one or more sensors to acquire data. For example, the CO2 sensor and/or the (VOC) sensor discussed herein may be activated to acquire data for those sensors. Such activation may occur prior to airflow being delivered to the patient. In some examples, the controller may activate the CO2 sensor and/or the (VOC) sensor when a reading from an airflow sensor (e.g., also provided in the adapter) indicates that no airflow is being delivered to the patient.

The components of adapter 9400′ may all be provided on the same PCB 9500, or separately. For example, some or all sensors may be provided separately from the PCB 9500 and electrically coupled to the PCB by a lead line or the like. In other examples, such as shown in FIG. 38, sensors (e.g., pressure sensor 9490) may be provided on the PCB. Note that the antenna may be partially (or wholly) integrated into the PCB 9500 in that it may be electrically connected to the transceiver 9300 that is provided on the PCB 9500.

Controller 9502 may provide different types of functionality according to various example embodiments. In some examples, the functionality of the controller 9502 may include pre or post processing of data that is acquired from the sensors and/or the tag 9200. For example, various filtering, averaging, and/or other processing may be performed by the controller 9502. In other examples, the functionality of the controller 9502 may be more limited in that data from the sensors is communicated back to RPT device 4000 for processing by the controller 4230 thereon. Accordingly, in some examples, the controller 9502 may be responsible for processing or analysing acquired and in other examples may be responsible for operating the various components of the adapter 9400′ without performing such processing or analysis. Rather, such processing may be performed by the RPT device 4000 and/or external systems 9520.

As noted herein, rather than custom mechanical connections (e.g., snap-fit pinch arms), each of the proximal end 9420 and the distal end 9430 of the air adapter tube 9400 may include an iso-taper configured to allow the air adapter tube 9400 to connect between any patient interface and air delivery tube, i.e., air adapter tube 9400 not limited for connection with a patient interface or an air delivery tube having compatible mechanical connectors. In addition, rather than a custom electrical connection to a heated air delivery tube (e.g., leadframe with electrical contacts) for power/signal transmission, the distal end 9430 may be provided without any leadframe/electrical contacts, i.e., air adapter tube 9400 not limited for connection with an air delivery tube having compatible electrical connectors. In such arrangement, the air adapter tube 9400 may include a power source (e.g., rechargeable battery) configured to provide power to the one or more sensors, FPC, antenna, etc. Also, any signal transmission may be provided by the antenna, e.g., NFC, Bluetooth, and the like. Such air adapter tube 9400 would provide an agnostic tube configured to be compatible with different patient interfaces and air delivery tubing, i.e., not limited to a particular patient interface, air delivery tubing, or system.

In the illustrated example, the air adapter tube 9400 is in the form of a short tube, e.g., a length between about 5-15 cm, e.g., 8.5-10 cm. In an alternative example, the air adapter tube may be in the form of a ring-type adapter, e.g., in the form of a cuff or ring similar to the size of the proximal end 9420 (e.g., 1-4 cm). Such ring-type adapter may comprise the FPC, one or more sensors, antenna in a cuff configured for connection between a patient interface and air delivery tubing. As described herein, such ring-type adapter may include a power source (e.g., rechargeable battery), allowing such ring-type adapter to be agnostic configured to be compatible with different patient interfaces and air delivery tubing.

### 5.5.1 Multi-Directional Antennas

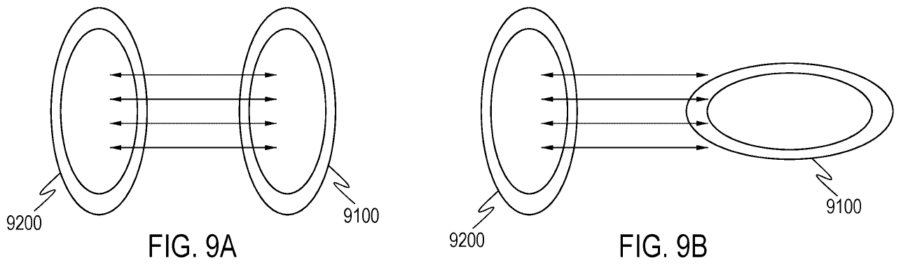

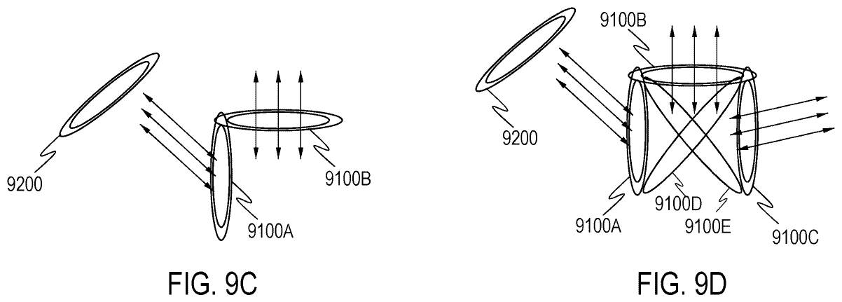

FIGS. 9A-9D illustrate various configurations of one or more antennas 9100 and one or more tags 9200. Because the antenna 9100 and the tag 9200 are inductances mutually coupled by a magnetic field, the antenna 9100 and tag 9200 may be configured within the RFID system 9000 to optimize a transfer of energy efficiently through the magnetic field. For example, the antenna 9100 and the tag 9200 may be configured to optimize the reading distance between the antenna 9100 and the tag 9200. In a less-optimized relative orientation, the reading distance may be affected between antenna 9100 and tag 9200.

FIG. 9A depicts a first configuration, in which the antenna 9100 is oriented substantially parallel relative to the tag 9200. For example, the antenna 9100 may be positioned to face the tag 9200. This configuration may optimize the magnetic field between the antenna 9100 and tag 9200, thus allowing for an increased reading distance between the antenna 9100 and tag 9200. For example, the antenna 9100 and tag 9200 may be spaced further apart from one another and still operate in the RFID system 9000. This configuration may be compared to a less-optimized second configuration, shown in FIG. 9B.

FIG. 9B depicts a second configuration, in which the antenna 9100 and the tag 9200 are offset relative to one another by approximately 90 degrees. This second configuration may be less desirable due to a disparate reading angle between the antenna 9100 and the tag 9200. For example, the ability for the antenna 9100 to read the tag 9200 from this angle may be decreased due, at least in part, to the disparate reading angle. Thus, the magnetic field between the antenna 9100 and the tag 9200 may not be optimized in this second configuration. Accordingly, the reading distance between the antenna 9100 and the tag 9200 is decreased in this configuration compared to the configuration of FIG. 9A.

Accordingly, the optimization of the magnetic field between the antenna 9100 and the tag 9200, and thus the ability for the antenna 9100 to read the tag 9200 at a given distance, is directly proportionate to the angle at which the antenna 9100 and the tag 9200 are oriented relative to one another. For example, the magnetic field between the antenna 9100 and the tag 9200 may be optimized when there is less of an angle between the antenna 9100 and the tag 9200, as shown in FIG. 9A. The magnetic field between the antenna 9100 and the tag 9200 may decrease as the antenna 9100 and/or the tag 9200 are angled relative to one another, as shown in FIG. 9B.

In the context of the systems 8000 described above, the angle between a tag 9200 incorporated into a patient interface and the antenna 9100 incorporated into an accessory or air circuit 4170 may depend on a number of factors. For example, if the antenna 9100 is incorporated into the air circuit 4170, the angle of attachment of air circuit 4170 relative to the patient interface 3000 may differ greatly for different types of patient interfaces. As shown in FIG. 4P, and described above, numerous configurations of patient interfaces are possible. Further, where the tag 9200 is incorporated on the patient interface 3000 may also impact the relative orientation of tag 9200 and the antenna 9100 when air circuit 4170 is coupled to the patient interface 3000.

The multi-directional antenna configurations depicted in FIGS. 9C and 9D may be configured to compensate for the differences in angles of attachment between the air circuit 4170 and the patient interface 3000 and may facilitate reading at a broader range of angles and mitigate one or more of the impacts on reading distance described above in reference to FIG. 9B. The disclosed multi-angle antenna configurations may be designed to arrange a plurality of antennas in a three-dimensional shape to create an efficient reading orientation between the antenna 9100 and the tag 9200 across a variety of different patient interface types.

In the configuration depicted in FIG. 9C, two antennas 9100A and 9100B may be angled relative to one another and oriented such that a first antenna 9100A and a second antenna 9100B face different directions. Accordingly, the first antenna 9100A and the second antenna 9100B are configured to provide reading angles in two directions. The orientation of the tag 9200 in FIG. 9C may represent the position of the tag 9200 on a patient interface or accessory, and the orientations of the first antenna 9100A and second antenna 9100B relative to the tag 9200 may represent the position of first antenna 9100A and second antenna 9100B on air circuit 4170 when attached to the patient interface or accessory. In the configuration depicted in FIG. 9C, the relative orientations of first antenna 9100A and second antenna 9100B may resemble a “figure eight” to provide dual-direction reading angles. For example, the first antenna 9100A is configured to provide a first reading angle in a first direction, and second the antenna 9100B is configured to provide a second reading angle in a second direction. The reading directions relative to the tag 9200 are depicted by the arrows shown in FIG. 9C. In this way, a tag 9200 may be efficiently read from multiple directions. For example, the tag 9200 may be read by the first antenna 9100A or the second antenna 9100B, depending on where the tag 9200 is positioned relative to first antenna 9100A and the second antenna 9100B. In the arrangement of FIG. 9C, the tag 9200 may be read by the first antenna 9100A, as the first antenna 9100A may be the antenna with a reading angle that is oriented more in line with the tag 9200.

First antenna 9100A and second antenna 9100B may be offset relative to one another by an angle A. Angle A may be approximately 90 degrees. Alternatively, angle A may be greater than 90 degrees or less than 90 degrees. For example, angle A may be approximately 30 degrees to approximately 120 degrees, or any other suitable angle.

In some instances, the first antenna 9100A, the second antenna 9100B, and the tag 9200 may be set to a frequency of 13.56 MHz. If the resonant frequency is in proximity to the reader carrier frequency (13.56 MHz), the power transfer between reader and tag as well as the communication distance may be increased. Although a frequency of 13.56 MHz is used as an example, any other suitable frequency may be utilized, as described above.

FIG. 9D depicts another multi-directional antenna arrangement that may include additional antennas in a more complex three-dimensional shape. In this configuration, a first antenna 9100A, a second antenna 9100B, a third antenna 9100C, a fourth antenna 9100D, and a fifth antenna 9100E are oriented to provide reading angles in multiple directions. The orientation of the tag 9200 in FIG. 9D may represent the position of tag 9200 on a patient interface or accessory, and the orientations of the antennas 9100A-9100E may represent the position of the antennas 9100A-9100E on air circuit 4170 when attached to the patient interface or accessory. Although five antennas are shown, more antennas may be used to increase the number of reading directions, or fewer antennas may be used to decrease the number of reading directions. For example, three, four, six, seven, or eight or more antennas may be used.

The embodiment of FIG. 9D may accommodate use with a variety of patient interfaces having a variety of different attachment orientations of the air circuit 4170 relative to the patient interface 3000 and the tag 9200. Further, in some aspects, as described above, the air circuit 4170 may be configured to move or swivel relative to the patient interface 3000 when connected to the patient interface 3000. For example, there may be a swivel in the air circuit 4170, or a patient interface connector. Because the antenna arrangement of FIG. 9D incorporates a plurality of antennas oriented so as to face a plurality of different directions, even when the air circuit 4170 moves relative to the patient interface when connected to the patient interface, a portion of the multi-directional antenna emitted field may be received by the tag 9200. As the air circuit 4170 moves, different antennas in the multi-directional configuration may be used to read the tag 9200, according to whichever antenna is most closely aligned with the tag 9200.

In some instances, the first antenna 9100A, the second antenna 9100B, the third antenna 9100C, the fourth antenna 9100D, and the fifth antenna 9100E may be set to a frequency of 13.56 MHz. If the resonant frequency is in proximity to the reader carrier frequency (13.56 MHz), the power transfer between reader and tag as well as the communication distance may be increased. Although a frequency of 13.56 MHz is used as an example, any other suitable frequency may be utilized, as described above.

FIGS. 10A-10D illustrate a variety of configurations between a variety of patient interfaces and an air circuit. The configurations of antennas and tags shown in FIGS. 9C-9D may be utilized, for example, to create efficient reading orientations in the configurations shown in FIGS. 10A-10D.

For example, FIG. 10A illustrates a frame 10000 of a tube-up nasal patient interface. In some embodiments, the frame 10000 may be utilized with patient interface 3000, shown in FIGS. 8A and 8B. The frame 10000 is configured such that air circuit 4170 is coupled to the frame 10000 at or near the top of a user's head, for example, via a joint 10002. Air circuit 4170 may be rotatable coupled relative to the frame 10000 via joint 10002. The joint 10002 may be a swivel joint, thus permitting the air circuit 4170 to rotate relative to frame 10000. As shown, the frame 10000 and the air circuit 4170 are angled relative to one another at approximately a 90 degree angle. Accordingly, the antenna 9100, which is fixed in or on a proximal portion of the air circuit 4170, may be angled relative to the tag 9200 fixed in or on the frame 10000.

As discussed above, the orientation of the antenna 9100 and the tag 9200 may impact the ability of the antenna 9100 to read the tag 9200. Thus, in the configuration shown in FIG. 10A, if a single, two-dimensional antenna 9100 is used as shown, there may be a decreased reading efficiency between the antenna 9100 and the tag 9200. Using two or more antennas 9100 in the form of a multi-dimension antenna, e.g., as shown in FIGS. 9C and 9D, may increase the reading efficiency between the antenna 9100 and the tag 9200 in this configuration. For example, as shown in FIG. 9D, a first antenna 9100A, a second antenna 9100B, a third antenna 9100C, a fourth antenna 9100D, and/or a fifth antenna 9100E may be utilized in this configuration. The use of multiple antennas arranged in a three-dimensional configuration may promote efficient reading of the tag 9200, regardless of the orientation of the air circuit 4170 relative to the frame 10000 when connected.

FIG. 10B illustrates an alternative configuration between the patient interface 3000 and the air circuit 4170, e.g., a tube-down nasal patient interface (according to any of the examples described herein). The air circuit 4170 may be coupled to patient interface via a joint 11000. The joint 11000 may permit rotation of the air circuit 4170, for example, about axis X. Depending on how the air circuit 4170 and the patient interface 3000 move relative to one another, use of a single, two-dimensional antenna 9100 as shown may lead to decreased reading efficiency in some relative configurations. Using two or more antennas 9100 in the form of a multi-dimensional antenna, e.g., as shown in FIGS. 9C and 9D, may increase the reading efficiency between antenna 9100 and tag 9200. For example, as shown in FIG. 9D, a first antenna 9100A, a second antenna 9100B, a third antenna 9100C, a fourth antenna 9100D, and/or a fifth antenna 9100E may be utilized in this configuration. The use of multiple antennas arranged in a three-dimensional configuration may promote efficient reading of the tag 9200, regardless of the orientation of the air circuit 4170 relative to the patient interface 3000.

FIGS. 10C and 10D illustrate another alternative configuration between a patient interface 3000 and the air circuit 4170. For example, in this configuration, the patient interface 3000 is a tube-down full-face patient interface. The air circuit 4170 may be coupled to patient interface via a joint 12000. The joint 12000 may be a swivel joint and thus permit rotation of air circuit 4170. For example, the joint 12000 may permit rotation about an axis Y of the patient interface 3000, as well as movement of the air circuit 4170 relative to the patient interface 3000. FIGS. 10C and 10D depict different positions of air conduit relative to the patient interface 3000 and the tag 9200, e.g., depending on the different positions a patient may be in when wearing the patient interface 3000. As the patient moves during use, the air circuit 4170 may change orientations unpredictably. In the positions shown in FIGS. 10C and 10D, the antenna 9100 and the tag 9200 may be offset from one another. For example, the antenna 9100 and the tag 9200 may be oriented such that the antenna 9100 and the tag 9200 are angled relative to one another. As previously described, an angle between the antenna 9100 and the tag 9200 may result in a less-optimized reading orientation. As described above in reference to FIGS. 10A and 10B, using two or more antennas 9100 in the form of a multi-dimension antenna, e.g., as shown in FIGS. 9C and 9D, may increase the reading efficiency between the antenna 9100 and the tag 9200. For example, as shown in FIG. 9D, a first antenna 9100A, a second antenna 9100B, a third antenna 9100C, a fourth antenna 9100D, and/or a fifth antenna 9100E may be utilized in this configuration. The use of multiple antennas arranged in a three-dimensional configuration may promote efficient reading of the tag 9200, regardless of the orientation of the air circuit 4170 relative to the patient interface 3000 when connected.

Each embodiment discussed herein may enable the user to detect the identification of the patient interface or accessory being used. In such a way, information regarding the patient interface, the accessory, the patient, or the therapy prescribed may be transmitted via RFID. Each embodiment may help to increase patient use and/or patient comfort, among other things.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

### 5.6 Reference Signs List

| Reference | |
|---|---|
| No. | Reference Label |
| 1000 | patient |
| 1100 | bed partner |
| 3000 | patient interface |
| 3000′ | patient interface |
| 3000-1 | tube up nose and mouth patient interface |
| 3000-2 | nose and mouth patient interface |
| 3000-3 | patient interface |
| 3000-4 | nose only patient interface |
| 3002 | shell |
| 3002A | first portion |
| 3002B | second portion |
| 3002C | third portion |
| 3002D | fourth portion |
| 3006 | cushion |
| 3008 | opening |
| 3012A | first nasal cushion |
| 3012B | second nasal cushion |
| 3014A | first hole |
| 3014B | second hole |
| 3016 | lumen |
| 3018 | tubular portion |
| 3018A | first portion |
| 3018B | second portion |
| 3018C | third portion |
| 3020A | first side |
| 3020B | second side |
| 3050-1 | cushion |
| 3050-2 | cushion |
| 3100 | seal-forming structure |
| 3200 | plenum chamber |
| 3200-1 | plenum chamber |
| 3200-2 | plenum chamber |
| 3254 | plenum chamber inlet port |
| 3254-2 | plenum chamber inlet ports |
| 3266-1 | groove |
| 3266-2 | groove |
| 3300 | structure |
| 3302 | headgear |
| 3302-1 | headgear |
| 3302-2 | headgear |
| 3304-1 | inferior strap |
| 3305-1 | superior strap |
| 3306-1 | magnetic member |
| 3307-1 | rear strap |
| 3307-2 | rear strap |
| 3308-2 | intermediate section |
| 3309-2 | slit |
| 3310 | strap |
| 3320 | tab |
| 3328 | concertina structure |
| 3332 | inlet |
| 3340 | rigidiser arm |
| 3342 | first end |
| 3344 | second end |
| 3350 | tube |
| 3351 | conduit sleeve |
| 3352 | superior opening |
| 3354 | inferior extension |
| 3356 | connection member |
| 3362 | extendable concertina structure |
| 3363 | non-extendable tube sections |
| 3370-1 | magnets |
| 3380 | four-point arm sleeve |
| 3380-1 | sleeve |
| 3384 | inferior extension |
| 3386 | connection member |
| 3388-1 | inferior opening |
| 3394 | tabs |
| 3394-1 | tabs |
| 3400 | vent |
| 3402 | vent opening |
| 3402-1 | vent opening |
| 3402-2 | vent opening |
| 3404 | vent housing |
| 3408 | anterior surface |
| 3412 | posterior surface |
| 3416 | groove |
| 3448 | diffuser |
| 3450 | vent |
| 3456 | anterior surface |
| 3464 | gap |
| 3500 | conduit connection structure |
| 3504 | connection structure |
| 3600 | connection port |
| 3605 | clip channel |
| 3610 | elbow |
| 3700 | forehead support |
| 3800 | patient interface |
| 4000 | RPT device |
| 4010 | external housing |
| 4020 | pneumatic block |
| 4100 | antenna |
| 4110 | air filter |
| 4112 | inlet air filter |
| 4114 | outlet air filter |
| 4120 | muffler |
| 4122 | inlet muffler |
| 4124 | outlet muffler |
| 4140 | pressure generator |
| 4142 | blower |
| 4144 | motor |
| 4160 | anti-spill back valve |
| 4170 | air circuit |
| 4170A | proximal portion |
| 4170B | proximal-most surface |
| 4170C | projection |
| 4171 | cuff portion |
| 4172 | cover |
| 4172A | proximal face |
| 4173 | indicator |
| 4174 | feature |
| 4175 | electrical connector |
| 4176 | opening |
| 4177 | pinch arms |
| 4177B | button portion |
| 4177C | catch portion |
| 4178 | lumen |
| 4179 | internal surface |
| 4180 | supplementary gas |
| 4181 | tubular end portion |
| 4185 | clip member |
| 4189 | recess |
| 4190 | protrusion |
| 4192 | circuit board |
| 4192A | proximal portion |
| 4192B | distal portion |
| 4194 | bridge |
| 4195 | surface |
| 4196 | platform |
| 4198 | opening |
| 4199 | encapsulation |
| 4202 | PCBA |
| 4210 | power supply |
| 4220 | input device |
| 4230 | central controller |
| 4232 | clock |
| 4240 | therapy device controller |
| 4250 | one or more protection circuits |
| 4260 | memory |
| 4270 | transducer |
| 4272 | pressure sensor |
| 4274 | flow rate sensor |
| 4276 | motor speed transducer |
| 4280 | data communication interface |
| 4282 | remote external communication network |
| 4284 | local external communication network |
| 4286 | remote external device |
| 4288 | local external device |
| 4290 | output device |
| 4292 | display driver |
| 4294 | display |
| 4300 | algorithms |
| 4310 | pre-processing module |
| 4312 | interface pressure estimation algorithm |
| 4314 | vent flow rate estimation algorithm |
| 4316 | leak flow rate estimation algorithm |
| 4318 | respiratory flow rate estimation algorithm |
| 4320 | therapy engine module |
| 4321 | phase determination algorithm |
| 4322 | waveform determination algorithm |
| 4323 | ventilation determination algorithm |
| 4324 | inspiratory flow limitation determination algorithm |
| 4325 | hypopnea determination algorithm |
| 4326 | snore determination algorithm |
| 4327 | airway patency determination algorithm |
| 4328 | target ventilation determination algorithm |
| 4329 | therapy parameter determination algorithm |
| 4330 | therapy control module |
| 4340 | one or more methods |
| 5000 | humidifier |
| 6320 | conduit |
| 8000 | respiratory therapy system |
| 9000 | RFID system |
| 9100 | antenna |
| 9100A | first antenna |
| 9100B | second antenna |
| 9100C | third antenna |
| 9100D | fourth antenna |
| 9100E | fifth antenna |
| 9200 | tag |
| 9300 | transceiver |
| 9400 | adapter |
| 9400′ | adapter |
| 9410 | tubular body |
| 9412 | tube wall |
| 9414 | electrical conductor |
| 9416 | helical rib |
| 9420 | proximal end |
| 9430 | distal end |
| 9432 | cuff portion |
| 9433 | indicator |
| 9434 | electrical connector |
| 9435 | electrical contact |
| 9436 | recess portion |
| 9437 | opening |
| 9439 | projection |
| 9440 | cuff portion |
| 9442 | recess |
| 9442U | upper recessed portion |
| 9442S | side recessed portion |
| 9445 | tubular end portion |
| 9446 | flange |
| 9447 | annular space |
| 9450 | clip member |
| 9452 | lug |
| 9454 | pinch arms |
| 9454B | button portion |
| 9454C | catch portion |
| 9456 | connecting portion |
| 9460 | first adapter element |
| 9462 | body portion |
| 9464 | tubular projection |
| 9465 | retaining member |
| 9466 | sensor support |
| 9467 | port |
| 9468 | vent hole |
| 9468.1 | first end |
| 9468.2 | second end |
| 9469b1 | raised bead |
| 9469b2 | raised bead |
| 9470 | second adapter element |
| 9471 | first end |
| 9472 | second end |
| 9480 | FPC |
| 9481 | first end |
| 9481o | opening |
| 9482 | second end |
| 9482o | opening |
| 9484 | joint area |
| 9490 | pressure sensor |
| 9492 | temperature sensor |
| 9494 | bridge |
| 9500 | printed circuit board (PCB) |
| 9502 | controller |
| 9504 | sensor(s) |
| 9506 | memory |
| 9508 | transceiver |
| 9520 | external computer system |
| 10000 | frame |
| 10002 | joint |
| 11000 | joint |
| 12000 | joint |

1-74. (canceled)

75. An air adapter tube for diagnosis and/or treatment of a respiratory disorder, comprising:
a tubular body configured to transport pressurized treatment air between a proximal end of the tubular body and a distal end of the tubular body, the proximal end being configured to connect to a patient interface, and the distal end being configured for connection to an air delivery tube; and
a sensor being configured to generate a signal indicative of sleeping position of a patient, wherein the sensor includes an accelerometer.

76. The air adapter tube according to claim 75, wherein the tubular body comprises a flexible material.

77. The air adapter tube according to claim 75, wherein an internal diameter of the tubular body is between about 10-15 mm.

78. The air adapter tube according to claim 75, wherein the tubular body includes a helix.

79. The air adapter tube according to claim 75, wherein the proximal end includes a mechanical connector configured to connect to the patient interface.

80. The air adapter tube according to claim 75, further comprising a flexible printed circuit at the proximal end supporting the sensor.

81. The air adapter tube according to claim 80, wherein the flexible printed circuit includes linear sections to support electronic components and at least one flexible bent portion between the linear sections.

82. The air adapter tube according to claim 81 wherein the one of the electronic components is a pressure sensor, and another of the electronic components is the accelerometer.

83. The air adapter tube according to claim 75, further comprising an antenna, wherein the antenna is configured to wirelessly transmit collected sensor data to an external device, including a smart phone or a flow generator.

84. The air adapter tube according to claim 83, further comprising at least one wire connection point that connects the antenna to at least one wire extending along and from the tubular body.

85. The air adapter tube according to claim 83, wherein the antenna is a multi-directional antenna comprising a plurality of antennas oriented at different angles relative to one another.

86. The air adapter tube according to claim 75, further comprising a first adapter element at the proximal end, the first adapter element supporting the sensor.

87. The air adapter tube according to claim 75, further comprising an electrical connector provided at the distal end of the tubular body, the electrical connector configured to electrically connect to a corresponding electrical connector of a heated air delivery tube.

88. The air adapter tube according to claim 75,
wherein the sensor includes a plurality of sensors including at least a first sensor and a second sensor,
wherein the first sensor is configured to generate, during therapy, a signal based on air passing at the proximal end for diagnosis and/or treatment of a respiratory disorder,
wherein the second sensor is configured to generate a signal indicative of a breath of and/or a biomarker of gas exhaled by the patient prior to therapy being applied.

89. An apparatus for diagnosing and/or treating a patient with a respiratory disorder, the apparatus comprising:
the air adapter tube according to claim 75;
a flow generator configured to generate pressurized breathable air for the patient, wherein the flow generator is configured to be controlled based on output from the sensor;
a patient interface configured to seal with airway of the patient; and
an air delivery tube to deliver the pressurized breathable air from the flow generator to towards the patient interface.

90. A system comprising:
the air adapter tube of claim 75; and
at least one hardware processor that is configured to perform operations comprising:
changing at least one therapy parameter based on the generated signal of the sensor,
controlling when the sensor is configured to sense a physical quantity,
causing a flow generator to adjust a generated pressure based on pressure data obtained from a pressure sensor of the air adapter tube, or
causing a flow generator to adjust a generated pressure based on data obtained from a CO₂ sensor of the air adapter tube.

91. The system of claim 90, wherein the sensor is configured to be controlled based on a determined position of a patient.

92. The system of claim 90, wherein the sensor is configured to be controlled to sense the physical quantity prior to positive air pressure being delivered to the air adapter tube or based on determination that there is no positive air pressure being delivered to the air adapter tube.

93. An air adapter tube for diagnosis and/or treatment of a respiratory disorder, comprising:
a tubular body, wherein a length of the tubular body is between about 8.5-10 cm;
a proximal end and a distal end, wherein the proximal end is configured to removably couple to a patient interface, and the distal end is configured to removably couple to an air delivery tube;
a sensor configured to generate a signal based a sensed physical quantity; and
an antenna configured to receive data from an RFID tag.

94. An air adapter tube for diagnosis and/or treatment of a respiratory disorder, comprising:
a tubular body configured to transport pressurized treatment air between a proximal end of the tubular body and a distal end of the tubular body, the proximal end being configured to connect to a patient interface, and the distal end being configured for connection to an air delivery tube;
a flexible printed circuit at the proximal end which includes linear sections to support electronic components and at least one flexible bent portion between the linear sections; and
a plurality of sensors being configured to generate a signal based on air flow at the proximal end for diagnosis and/or treatment of a respiratory disorder, wherein one of the plurality of sensors is a pressure sensor, and another of the plurality of sensors is an accelerometer.
